SEQUENCE LISTING (19 sequences)

SEQ ID NO: 1 - DNA, 20 bp, Artificial Sequence
  sgRNA for TRAC
tctctcagctggtacacggc

SEQ ID NO: 2 - DNA, 21 bp, Artificial Sequence
  TRAC forward primer
ttgatagcttgtgcctgtccc

SEQ ID NO: 3 - DNA, 21 bp, Artificial Sequence
  TRAC reverse primer
ggcaaacagtctgagcaaagg

SEQ ID NO: 4 - DNA, 600 bp, Artificial Sequence
  Terminal CRISPR long terminal repeat
agagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgatggaagacctgttttagagctagaaatagcaagttaaaataaggctagtccgttatcaacttgaaaaagtggcaccgagtcggtgctttttttctagactgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataactt

SEQ ID NO: 5 - DNA, 187 bp, Artificial Sequence
  U6 promoter region sequenced in Example 1
aggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtg

SEQ ID NO: 6 - DNA, 187 bp, Artificial Sequence
  U6 promoter region sequenced in Example 1
  misc_feature (73)..(73): n = unknown nucleotide
  misc_feature (131)..(131): n = unknown nucleotide
  misc_feature (133)..(133): n = unknown nucleotide
  misc_feature (162)..(162): n = unknown nucleotide
aggctgttagagagataattagaattaatttgactgtaaacacaaagatattagtacaaaatacgtgacgtanaaagtaataatttcttgggtagtttgcagttttaaaattatgttttaaaatggactancntatgcttaccgtaacttgaaagtatttcnatttcttggctttatatatcttgtg

SEQ ID NO: 7 - protein, 62 aa, Artificial Sequence
  U6 promotor sequence determined in Example 1
  MISC_FEATURE (3)..(3): x = unknown amino acid
  MISC_FEATURE (7)..(7): x = unknown amino acid
  MISC_FEATURE (9)..(9): x = unknown amino acid
  MISC_FEATURE (24)..(24): x = unknown amino acid
  MISC_FEATURE (40)..(40): x = unknown amino acid
GCXRDNXNXFDCKHKDISTKYVTXKVIISWVVCSFKIMFXNGLSYAYRNLKVFRFLGFIYLV

SEQ ID NO: 8 - DNA, 70 bp, Artificial Sequence
  U6 promoter sequence determined in in Example 1
  misc_feature (7)..(7): n = unknown nucleotide
  misc_feature (65)..(65): n = unknown nucleotide
  misc_feature (67)..(67): n = unknown nucleotide
gacgtanaaagtaataatttcttgggtagtttgcagttttaaaattatgttttaaaatggactancntat

SEQ ID NO: 9 - DNA, 245 bp, Artificial Sequence
  Sequence determined in Example 1
  misc_feature (1)..(29): gRNA scaffold
  misc_feature (30)..(50): HIV-deltaU3
  misc_feature (51)..(146): HIV- R region
  misc_feature (147)..(228): HIV- U5
  misc_feature (232)..(245): HIV - PBS
tggcaccgagtcggtgctttttttctagactgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacaggg

SEQ ID NO: 10 - DNA, 316 bp, Artificial Sequence
  Sequence determined in Example 1
  misc_feature (1)..(29): gRNA scaffold
  misc_feature (30)..(50): HIV-deltaU3
  misc_feature (51)..(146): HIV - R region
  misc_feature (147)..(228): HIV-U5
  misc_feature (232)..(249): HIV - PBS
  misc_feature (282)..(316): HIV - Psi
tggcaccgagtcggtgctttttttctagactgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggca

SEQ ID NO: 11 - DNA, 286 bp, Artificial Sequence
  Sequence determined in Example 1
  misc_feature (1)..(20): HIV-deltaU3
  misc_feature (21)..(116): HIV - R region
  misc_feature (117)..(190): HIV - U5
  misc_feature (202)..(219): HIV - PBS
  misc_feature (252)..(286): HIV - Psi
tgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggca

SEQ ID NO: 12 - protein, 105 aa, Artificial Sequence
  Sequence determined in Example 1
  MISC_FEATURE (9)..(9): Xaa =  unknown amino acid
  MISC_FEATURE (38)..(38): Xaa =  unknown amino acid
  MISC_FEATURE (57)..(57): Xaa =  unknown amino acid
  MISC_FEATURE (60)..(60): Xaa =  unknown amino acid
  MISC_FEATURE (75)..(75): Xaa =  unknown amino acid
GTESVLFFXTAFCLYWVSLVRPDLSLGALWLTREPTAXASIKLALSASSSVCPSVVXLWXLEIPQTLLVSVENLXQWRPNRDLKAKGKPEELSRRRTRLAEARTA

SEQ ID NO: 13 - DNA, 70 bp, Artificial Sequence
  Sequence determined in Example 1
agatctgagcctgggagctctctggctaactagggaacccactgcttaagcctcaataaagcttgccttg

SEQ ID NO: 14 - DNA, 70 bp, Artificial Sequence
  Sequence determined in Example 1
agaggagctctctcgacgcaggactcgggttgctgaagcgggcacggcaagaggcgaggggcggcgactg

SEQ ID NO: 15 - DNA, 332 bp, Artificial Sequence
  Sequence determined in Example 1
  misc_feature (1)..(47): HIV - deltaU3
  misc_feature (48)..(143): HIV - R region
  misc_feature (144)..(225): HIV - U5
  misc_feature (229)..(246): HIV - PBS
  misc_feature (279)..(332): HIV - Psi
taattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcggggcggcgacaagaggcga

SEQ ID NO: 16 - DNA, 332 bp, Artificial Sequence
  Sequence determined in Example 1
  misc_feature (1)..(47): HIV - deltaU3
  misc_feature (3)..(5): n = unknown nucleotide
  misc_feature (7)..(8): n = unknown nucleotide
  misc_feature (48)..(143): HIV - R region
  misc_feature (144)..(225): HIV - U5
  misc_feature (229)..(246): HIV - PBS
  misc_feature (279)..(332): HIV - Psi
tannncnntcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcggggcggcgacaagaggcga

SEQ ID NO: 17 - protein, 112 aa, Artificial Sequence
  Sequence determined in Example 1
  MISC_FEATURE (37)..(37): Xaa = unknown amino acid
  MISC_FEATURE (57)..(57): Xaa = unknown amino acid
  MISC_FEATURE (60)..(60): Xaa = unknown amino acid
  MISC_FEATURE (75)..(75): Xaa = unknown amino acid
NSLPTKTRSAFCLYWVSLVRPDLSLGALWLTREPTAXASIKLALSASSSVCPPSVVXLWXLEIPQTLLVSVENLXQWRPNRDLKAKGKPEELSRRRTRLAEARTARGEGRRL

SEQ ID NO: 18 - DNA, 630 bp, Homo sapiens
  misc_feature (174)..(180): Splice acceptor site
  misc_feature (181)..(453): EX1
  misc_feature (435)..(438): PAM
  misc_feature (439)..(458): Cas9-CD donor site guide
  misc_feature (453)..(459): Splice donor site
cccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtcca

SEQ ID NO: 19 - protein, 90 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSP

(19) United States
(12) Patent Application Publication
(10) Pub. No.: US 2020/0087681 A1
(43) Pub. Date: Mar. 19, 2020

(54) THERAPEUTIC CELLS

(71) Applicant: UCL Business PLC, London (GB)

(72) Inventors: Waseem Qasim, Woking (GB); Christos Georgiadis, London (GB); Roland Preece, London (GB); Ulrike Mock, Bergisch-Gladbach (DE); Lauren Nickolay, Kent (GB)

(21) Appl. No.: 16/470,933

(22) PCT Filed: Dec. 21, 2017
(86) PCT No.: PCT/GB2017/053862

§ 371 Date: Jun. 18, 2019

(30) Foreign Application Priority Data
Dec. 21, 2016 (GB) 1621874.5
Apr. 18, 2017 (GB) 1706101.1

(51) Int. Cl. C12N 15/86; C12N 7/00; C12N 15/11; C12N 9/22; C07K 16/28; A61K 35/17; C07K 14/735; C07K 14/705; C07K 14/725; C12N 5/0783; A61P 35/02

(52) U.S. Cl. CPC C12N 15/86; A61K 38/00; C12N 15/11; C12N 9/22; C07K 16/2803; A61K 35/17; C07K 14/70535; C07K 14/70517; C07K 14/70578; C07K 14/7051; C12N 5/0636; A61P 35/02; C12N 2740/15043; C12N 2310/20; C12N 2800/80; C07K 2317/24; C12N 2510/00; C07K 2319/30; C07K 2319/33; C07K 2319/02; C07K 2319/03; C12N 7/00

(57) ABSTRACT

The invention relates to therapeutic cells, and methods employed in their production.

### FIELD OF THE INVENTION

The invention relates to therapeutic cells and, particularly, methods employed in their production.

### BACKGROUND TO THE INVENTION

Cell therapy is therapy in which cellular material is introduced into a patient. Generally, intact, living cells are introduced to the patient. For example, T cells capable of fighting cancer cells via cell-mediated immunity may be introduced to a patient in the course of anti-cancer immunotherapy. Cells may also be introduced to a patient to treat virus infections such as Cytomegalovirus, Epstein Barr Virus or Adenonvirus, and to eradicate host immunity and support donor chimerism in the context of bone marrow transplantation

Therapeutic cells may be autologous or allogeneic in relation to the patient into which they are to be introduced. If allogeneic cells are used, consideration must be given to the consequences of potential HLA-mismatch between the donor and recipient. For all types of therapeutic cells, minimising human leukocyte antigen (HLA)-mismatch reduces rejection of the therapeutic cells by the patient, improving their longevity and therapeutic potential. Furthermore, it is particularly important to reduce HLA-mismatch for therapeutic immune cells or hematopoietic stem cells, due to their ability to cause graft versus host disease (GVHD) when transplanted to an HLA-mismatched patient. GVHD arises when the native T-cell receptor (TCR) of T cells in or arising from the donated tissue (the “graft”) recognise antigens in the recipient (the “host”) as foreign. Thus, transplanted T cells attack host cells and tissues, causing damage to the host organs.

Immune system cells and their precursors are often used in cell therapy because the immune responses they propagate can be harnessed against antigens of therapeutic interest, such as a tumour or viral antigen. T cells have particular utility because they naturally mediate powerful cytotoxic effects and have immunological ‘memory’ providing long term effects. Therapeutic T cells are often autologous—i.e. they are generated from the patient's own lymphocytes. This is effective but can be complex and has a number of limitations: (1) it may be difficult or impossible to generate a product from patient's own lymphocytes due to insufficient quantity or quality of lymphocytes consequent to disease or chemotherapy or high levels of leukemia in the circulation; (2) there may be insufficient time to generate an autologous T-cell product due to the course of the patient's illness; and (3) autologous production requires a bespoke product to be manufactured for each patient which makes manufacture costly.

An alternative approach is to generate an “off-the-shelf” T cell product from healthy lymphocytes from an allogeneic or partially HLA-mismatched donor. Using the off-the-shelf approach, production of the therapeutic T-cell product is independent of the patient. Furthermore, if the manufacturing process lends itself to economies of scale, the off-the-shelf approach may advantageously reduce the cost of production of the T-cell product. In addition, a bank of therapeutic T cells may be created, ready for use in any patient at any time.

To provide a safe off-the-shelf T cell product, it is necessary to produce “universal” therapeutic T cells. Universal T cells are T cells that may be introduced to any individual with no or minimal deleterious effect on the health of the individual. In particular, universal T cells have a reduced capacity to cause graft versus host disease (GVHD) when transplanted to a HLA-mismatched individual, compared to regular, non-universal T cells.

Given the wide variability of HLA types, it is very likely that any off-the-shelf T-cell product will be at least partially HLA-mismatched from the recipient. It is simply not feasible to have a HLA-matched, off-the-shelf T-cell product ready for every recipient in need thereof. As set out above, HLA mismatch is associated with GVHD. In order to be of widespread utility, an off-the-shelf, universal T-cell product must cause no or minimalGVHD when administered to a HLA-mismatched recipient.

HLA-mismatch may also be detrimental to the graft. As mentioned above, the graft may be rejected if immune cells in the host recognise antigens in the graft as foreign and attack grafted cells. Therefore, in order to be of widespread utility, an off-the-shelf, universal T-cell product must be subject to minimal or no rejection when administered to a HLA-mismatched recipient. This can be achieved either by removing HLA molecules, in particular class I HLA, from the surface of T cells or by targeting other genes that then render the cells resistant to the effects of lymphodepleting agents. One example of this is the disruption of CD52 on T cells to confer resistance to the monoclonal anti-CD52 antibody Alemtuzumab.

Therapeutic cells, including T cells, may comprise modifications associated with their therapeutic effect. For instance, a therapeutic cell may be modified to be targeted towards an antigen of interest, or to express a particular therapeutic molecule. Exogenous molecules (e.g. an antigen receptor or therapeutic molecule) can be introduced to a therapeutic cell by transfecting or transducing the therapeutic cell with a nucleic acid sequence or construct encoding the molecule (i.e. a transgene). Such transfection and transduction is well known in the art.

Furthermore, endogenous expression of a molecule by a therapeutic cell can be disrupted using genome editing techniques such as zinc finger nucleases (ZFNs), transcription activator-like effector nucleases (TALENs), Meganucleases and Clustered regularly interspaced short palindromic repeats (CRISPR)/Cas. These genome editing methods can disrupt a gene, entirely knocking out all of its output. However, ZFNs, TALENS and CRISPR/Cas can all introduce off-target gene disruptions, and cause unwanted chromosomal changes including translocations, additions and deletions. In addition, the genes required for these approaches typically have to be delivered separately to cells during their modification, for instance by electroporation with synthetic mRNA. Consequently, the resultant disruption or knockdown of the target gene in the cell is not linked to expression of a transgene provided concurrently. This means that sorting for transgene-expressing cells does not necessarily also sort for cells expressing the genome editing. Likewise, sorting for expression of genome editing genes does not necessarily sort for transgene-expression. Therefore, to select genome-edited, transgene-expressing cells, it is necessary to perform two different sorting steps, one to select for transgene-expressing cells and one to select for cells expressing the genome editing genes

In a particular type of cell therapy, therapeutic T cells may be engineered to direct their cytotoxic effects towards a particular antigen of interest. For example, T cells for killing tumour cells may be specific for a tumour antigen. T cells for killing virally-infected cells may be specific for a viral antigen that is present on the surface of infected cells. T cell specificity may directed by endogenous αμ T cell receptors, or via introduced recombinant αμ receptors or by chimeric antigen receptors. The latter usually incorporates a single chain variable fragment (scfv) derived from the antigen binding regions of an antibody, linked to transmembrane and intracellular activation domains.

Certain cells, notably Natural Killer cells but only a minority of T cell subsets, may also mediate antibody dependent cell cytotoxicity (ADCC) of a cell that is “tagged” by a specific antibody. In this way, the cytotoxicity cells may be harnessed towards other cells expressing a particular antigen, via an antibody specific for that antigen. Such signals are conveyed via binding of the Fc portion of IgG antibody by a transmembrane receptor (FcR) also known as CD16. To increase and enhance the ability of T cells to mediate cytotoxic effects in an antibody-dependent manner, levels of FcR expression in T cells can be increased.

Accordingly, there are several problems associated with the production of therapeutic cells. Firstly, it is desirable for therapeutic cells to be autologous or HLA-matched with the patient to which they will be introduced, but it is not always possible to obtain sufficient autologous/HLA-matched cells for this purpose. Secondly, the expression profile and/or genome of therapeutic cells often needs to be modified to optimise therapeutic activity, but existing mechanisms for this can be unreliable. Thirdly, to direct therapeutic cells (particularly T cells) against an antibody-tagged cell, it is necessary to equip the therapeutic cell with an efficient targeting and signaling molecule

### SUMMARY OF THE INVENTION

The present invention aims to overcome the problems associated with producing therapeutic cells set out above. In particular, the present disclosure provides a method of generating universal therapeutic T cells that may be introduced to HLA-mismatched, or partially HLA-mismatched, individuals with no or minimal deleterious effect. In particular, the present disclosure aims to provide a method of generating a universal T cell whose cytotoxicity may be harnessed by an antibody. In this way, a pool of therapeutic T cells could be generated that may be administered to any individual, and that may be used to target any antigen to which a antibody exists. The potential therapeutic applications of such a cell would be very broad. Furthermore, due to economies of scale, provision of a single, universal, cover-all T cell product would be more cost effective than provision multiple different T cell products tailored to a particular individual and a particular antigen.

To this end, the present disclosure relates to universal antibody dependent cord T cells (U-ACTs), and methods employed in their production. Unlike existing therapeutic T cells, the U-ACTs' cytotoxicity may be directed to any antigen for which there is any antibody. Furthermore, the U-ACTs of the disclosure have no or minimal capacity to cause GVHD following administration to an individual. The U-ACTs of the invention are subject to no rejection, or a minimal amount of rejection, when administered to a HLA-mismatched recipient. The U-ACTs of the disclosure are produced by a new and advantageous method, the steps of which have not previously been individually described.

To obtain T cells that may be modified to become U-ACTs, the inventors have developed a new method of isolating T cells from umbilical cord blood. T cells isolated from cord blood using this new method can also be used for other applications, e.g. to prepare therapeutic cells other than U-ACTs (such as the CD19−CAR expressing, TRAC deficient cells of the invention), or for use in research. Umbilical cord T cells are particularly suited for therapeutic uses because they harbour distinct molecular and cellular characteristics capable of supporting immunotherapeutic effects. They are almost entirely of a naïve phenotype, have extensive proliferative capacity, and can mediate potent anti-viral and anti-leukemic effects in the allogeneic transplant setting. In addition, in contrast to adult donor haematopoietic stem cell transplants, umbilical cord grafts are routinely undertaken with one or more HLA mismatches without notable exacerbations of GVHD or higher rates of rejection.

In more detail, the inventors have found that T cells may be isolated from a sample of cord blood cells by isolating cells that express CD62L from the sample. Positive selection for CD62L-expressing cells yields an unexpectedly pure population of cord blood T cells (i.e. a population containing an unexpectedly high proportion of cord blood T cells). In addition, by selecting for a marker other than those involved in T cell activation and expansion (e.g. CD3, T cell receptor (TCR)), the resultant population of cord blood T cells is “untouched” via receptors targeted for activation and expansion during downstream processing. This avoids the mechanism by which cord blood T cells are isolated having a deleterious effect on later activation and modification of the T cells. Such effects may include effects as activation-induced cell death and apoptosis.

In addition, the inventors have developed an improved way of modifying the genome of cells, such as therapeutic cells and T cells. Specifically, the inventors have devised an advantageous method for disrupting endogenous expression of a gene. Amongst other applications (see below), the method may be used to render a T cell universal, by disrupting expression of TCR and/or MHC Class I.

The method is a modified CRISPR method, known as “terminal CRISPR”. In terminal CRISPR, one or more CRISPR guide sequences targeting a gene to be disrupted (e.g. a gene associated with TCR or MEW Class I expression) are introduced to the cell using a lentiviral vector. For biosafety purposes, the vector may be produced by transient transfection of a split packaging system that includes a vector genome plasmid. The vector/vector genome plasmid comprises a 3′long terminal repeat region (3′LTR) comprising one or more promoter sequences operably linked to the sequence encoding one or more guide sequences. The LTR preferably comprises a H1 promoter sequence. The LTR preferably comprises a U6 promoter sequence. The LTR may comprise two or more different promoter sequences. For example, the LTR may comprise a H1 promoter sequence and a U6 promoter sequence, and optionally one or more other different promoter sequences. The LTR preferably comprises two or more sequences encoding a CRISPR guide sequence, each operably linked to a different promoter sequence. Inclusion of the promoter sequence(s) and guide sequence(s) in a LTR (of such a vector allows the promoter and guide sequence(s) to be duplicated during reverse transcription such that they become incorporated into both the 5′ and 3′ LTRs. Guide sequence expression is therefore doubled and genome editing is more efficient. Furthermore, by including the guide sequences and their promoters in the 3′LTR, interference with vector genome expression during vector manufacture or with transgene expression following transduction is avoided. Titre and expression comparable to conventional vectors is thereby achieved. A CRISPR CRISPR guided DNA modification enzyme, such as a cytidine deaminase or a CRISPR nuclease such as Cas9, is separately delivered by electroporation to the virally transduced T cells for transient guide direction scission effects. Provision of the CRISPR guided DNA modification enzyme can therefore be controlled separately from guide sequence expression, lending an extra degree of tunability to the cells.

The inventors have also found that T cell cytotoxicity can be harnessed to bring about antibody mediated cell cytotoxicity (ADCC) by introducing an engineered Fc-Receptor to the T cell. In particular, a chimeric FcR may be introduced that comprises (I) an extracellular domain that is capable of binding to a constant domain of an antibody and (II) a transmembrane domain and a cytoplasmic domain that are capable of supporting T cell activation. In this way, the T cell is targeted towards an antibody, and is activated by binding of the antibody by the FcR. An important advantage of the chimeric FcR over CARs is that a single cFcR platform can be combined with multiple therapeutic antibodies, and rather than having to generate multiple CARs each with a particular scFv receptors derived from specific antibodies. In a related aspect, NK cell cytotoxicity may be harnessed to bring about ADCC in the same way.

Using all of these methods in combination, a U-ACT expressing a FcR that is capable of activating the cell upon binding to a constant domain of an antibody, and having disrupted expression of TCR and MHC I may be produced. As set out above, the therapeutic applications for such a U-ACT are very broad.

Other combinations of the methods may also be employed to generate different cells. For instance, terminal CRISPR may be used to beneficially modify the genome of a different type of cell, for instance to reduce a side effect associated with administration of the cell to an individual, or to prolong cell survival, improve function and reduce exhaustion effects. Terminal CRISPR may be used to disrupt TCR expression and induce expression of a CAR in a T cell. Terminal CRISPR may be used to modify the genome of cord blood T cells advantageously isolated on the basis of their CD62L expression. The FcR described above may be introduced to such cord blood cells. The FcR above may be introduced to other cells whose genomes are modified by terminal CRISPR. The various methods of the disclosure may be combined in any combination, to tailor the resulting cells to the application for which they are intended.

Accordingly, the invention provides a method for delivering CRISPR guide sequences and a CRISPR guided DNA modification enzyme to a cell, comprising: (a) introducing one or more CRISPR guide sequences to said cell using a vector that comprises a 3′long terminal repeat region (LTR) containing one or more promoter sequences operably linked to the sequence encoding the said CRISPR guide sequence(s); and (b) separately delivering the CRISPR guided DNA modification enzyme to said cell of (a) by introducing into it a nucleic acid or protein sequence encoding said CRISPR guided DNA modification enzyme.

The invention also provides:
a vector that comprises a 3′ LTR comprising one or more promoter sequences operably linked to a sequence encoding one or more CRISPR guide sequences;
use of the vector of the invention to: (a) disrupt expression of TCR and/or MHC class 1 in a cell; (b) introduce a nucleic acid sequence encoding a FcR that comprises (I) an extracellular domain that is capable of binding to a constant domain of an antibody and (II) a transmembrane domain and a cytoplasmic domain that are capable of supporting T cell activation into a cell; (c) introduce a nucleic acid sequence encoding a CAR into a cell, optionally wherein the CAR is specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1 MUC1, PSMA, VEGF-R, Her2, or CAIX; (d) introduce a nucleic acid sequence encoding a CAR into a cell and to disrupt expression of TCR and/or MHC class 1 in the cell, optionally wherein the CAR is specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1 MUC1, PSMA, VEGF-R, Her2, or CAIX; (e) introduce a nucleic acid sequence encoding a rTCR into a cell and to disrupt expression of TCR in the cell; (f) introduce a nucleic acid sequence encoding a restriction factor into a cell and to disrupt expression of CCR5 in the cell, optionally wherein the restriction factor is TRIM5CypA; (g) disrupt expression of a locus controlling a gain of function mutation in a cell and to introduce a nucleic acid sequence encoding a replacement protein into the cell; (h) disrupt expression of a locus controlling a transgene silencing pathway in a cell, optionally wherein the vector comprises a nucleic acid sequence encoding a transgene silenced by the pathway; or (i) disrupt expression of a locus controlling a checkpoint inhibitor pathway in a cell and to introduce a nucleic acid sequence encoding a suicide into the cell;
a method for generating T cells that comprise a nucleic sequence encoding a CAR and have disrupted TCR and/or MHC class lexpression, comprising: (a) providing one or more T cells; (b) introducing into one or more of said T cells of (a) a nucleic acid sequence encoding a CAR; and (c) disrupting expression of TCR and/or MHC class 1 in said T cells of (b), wherein, in (c), the expression of TCR and/or MHC class 1 is disrupted by: (i) introducing one or more CRISPR guide sequences to said T cells of (b) using a vector that comprises a 3′ long terminal repeat region (LTR) comprising one or more promoter sequences operably linked to the sequence encoding said CRISPR guide sequence(s); and ii) separately delivering a CRISPR guided DNA modification enzyme to said T cells of (b) by introducing into them a nucleic acid or protein sequence encoding said CRISPR guided DNA modification enzyme;
a T cell that comprises a nucleic sequence encoding a CAR and has disrupted TCR expression;
a T cell that comprises a nucleic sequence encoding a CAR and has disrupted TCR expression for use in a method of treating a neoplastic condition, an autoimmune condition, an infectious condition, an inflammatory condition, a haematological disorder or a metabolic condition;
a method of treating a neoplastic condition, an autoimmune condition, an infectious condition, an inflammatory condition, a haematological disorder or a metabolic condition in a patient in need thereof, the method comprising administering to the patient an effective number of T cells of the invention; and
a pharmaceutical composition comprising the T cell of the invention.
   Also described herein is:
a method for generating universal antibody dependent cord T cells (U-ACTs), comprising: (a) providing a sample of cord blood; (b) separating cells that express CD62L from the sample, wherein the cells that express CD62L comprise cord blood T cells; (c) introducing into one or more of said cord blood T cells of (b) a nucleic acid sequence encoding an Fc-Receptor (FcR) that comprises (I) an extracellular domain that is capable of binding to a constant domain of an antibody and (II) a transmembrane domain and a cytoplasmic domain that are capable of supporting T cell activation; and (d) disrupting expression of T cell receptor and WIC class I in said cord blood T cells of (c), wherein, in (d), the expression of T cell receptor and/or MHC class 1 is disrupted by: (i) introducing one or more CRISPR guide sequences to said cord blood T cells of (c) using a vector that comprises a 3′ long terminal repeat region (LTR) comprising one or more promoter sequences operably linked to the sequence encoding the said CRISPR guide sequence(s); and (ii) separately delivering a CRISPR guided DNA modification enzyme to said cord blood T cells of (c) by introducing into them a nucleic acid or protein sequence encoding said CRISPR guided DNA modification enzyme;
a method for generating universal antibody dependent cord T cells (U-ACTs), comprising: (a) providing one or more cord blood T cells; (b) introducing into one or more of said cord blood T cells of (a) a nucleic acid sequence encoding an Fc-Receptor (FcR) that (I) an extracellular domain that is capable of binding to a constant domain of an antibody and (II) a transmembrane domain and a cytoplasmic domain that are capable of supporting T cell activation; and (c) disrupting expression of T cell receptor and MHC class I in said cord blood T cells of (b);
a method for generating cord blood T cells, comprising (a) providing a sample of cord blood; and (b) separating cells that express CD62L from the sample wherein the cells that express CD62L comprise one or more cord blood T cells;
a FcR that comprises (I) an extracellular domain that is capable of binding to a constant domain of an antibody and (II) a transmembrane domain and a cytoplasmic domain that are capable of supporting T cell activation;
dimer comprising two FcRs of the disclosure;
a nucleic acid sequence encoding a FcR of the disclosure;
vector comprising the nucleic acid sequence of the disclosure;
a cell comprising the nucleic acid of the disclosure or the vector of the disclosure;
a universal antibody dependent cord T cell (U-ACT) that comprises a FcR of the disclosure and has disrupted T cell receptor and MHC class I expression;
a pharmaceutical composition comprising a U-ACT of the disclosure;
a universal antibody dependent cord T cell (U-ACT) of the disclosure, for use in method of treatment of the human or animal body;
a universal antibody dependent cord T cell (U-ACT) of the disclosure, for use in a method of treating a neoplastic condition, an autoimmune condition, an infectious condition, an inflammatory condition, a haematological disorder, or a metabolic condition;
a universal antibody dependent cord T cell (U-ACT) of the disclosure, for use in a method of depleting immune cells and/or bone marrow cells in an individual;

a method of treating a neoplastic condition, an autoimmune condition, an infectious condition or an inflammatory condition in a patient in need thereof, the method comprising administering to the patient an effective number of U-ACTs of the disclosure.

### BRIEF DESCRIPTION OF THE FIGURES

FIG. 1: Schematic representation of the “Terminal CRISPR” lentiviral plasmid.

FIG. 2: Design of Terminal CRISPR long terminal repeat.

FIG. 3: Verification of terminal CRISPR duplication.

FIG. 4: Terminal CRISPR configuration maintains titre and transgene expression.

FIG. 5: T cell receptor knockout using Terminal CRISPR vectors expressing PGK-CAR19.

FIG. 6: Comparison data using Ribonucleoprotein delivery.

FIG. 7: Comparison data using alternative lentiviral-CRSIPR/Cas9 vectors and terminal U6 β₂m CRISPR vector.

FIG. 8: Titration of Cas mRNA in association with Terminal TRAC PGK CAR19 vectors.

FIG. 9: A) Terminal TRAC CRISPR/PGK CAR19 in cord blood T cells. B) Linking transgene to guide expression in a single vector results in a highly enriched product where TCR ko is used to select the cells at the end of manufacture.

FIG. 10: Terminal β₂m CRISPR/PGK CAR19 in cord blood T cells.

FIG. 11: Terminal β₂m CRISPR/PGK CAR19 in peripheral blood T cells.

FIG. 12: Schematic representation of the chimeric FcR (cFcR) vector plasmid.

FIG. 13: Schematic of cFcR mediated destruction of target cells.

FIG. 14: Demonstration of cFcR mediated binding of humanised IgG1 mAb

FIG. 15: cFcR mediated cytotoxicity of B cell tumour cells

FIG. 16: TCR depleted cFcR T cells.

FIG. 17: Generation of universal antibody dependent cytotoxic T cells (U-ACT).

FIG. 18. Density gradient separation of cord blood using the CliniMACS Prodigy. The Density Gradient Separation process on the CliniMACS prodigy was used to isolate lymphocytes from whole cord blood. Cord blood cells were analysed by Sysmex pre and post ficoll (density gradient separation). Results shown are from three individual cord blood donors.

FIG. 19. Density gradient separation of cord blood using the CliniMACS Prodigy. Frequency of lymphocyte subsets were analysed by flow cytometry on whole cord blood processed by density gradient separation using the CliniMACS Prodigy. A live gate was set based on the FSC-A/SSC-A profile of the cells and lymphocytes were identified based on the expression of CD45. CD45+ WBC were further analysed for expression of CD3 (T cells), CD14 (monocytes) CD56 (NK Cells) and CD20 (B Cells).

FIG. 20. Expansion and Transduction of Cord Blood T Cells Processed using Density Gradient Separation. Cord blood cells that had been processed using Density Gradient Separation were used to initiate the T cell Transduction process on the CliniMACS prodigy, which enables the automated expansion and transduction of T cells. Briefly, cells were activated within the closed tubing set of the CliniMACS Prodigy using TransAct activation reagent and after 48 hours the cells were transduced with a lentiviral vector encoding CD19− CAR. Cells were allowed to expand in the tubing set of the CliniMACS Prodigy for a total of 9 days. (A) Over the 9 days expansion period samples from the CliniMACS prodigy were taken and a WBC count obtained via Sysmex. Over the 9 days expansion period the cord blood cells had a total expansion of 2.4 fold. (B) At the end of the expansion process the cord blood cells were stained for antibodies against CD3 and CD45 and the T cell purity was analysed by flow cytometry. (C) Transduction efficiency of CD19−CAR was assessed by flow staining with a α-murine FAB antibody.

FIG. 21. Expression of CD62L on Whole Cord Blood. Whole cord blood was subjected to red blood cell lysis and stained with antibodies against CD3 and CD62L. The FSC-A/SSC-A profiled of CD3+CD62L+ and CD3−CD62L+ cells is shown to delineate the phenotype of the CD3−CD62L+ cells.

FIG. 22. Whole Cord Blood CD62L Selection using the CliniMACS Prodigy. Cord blood cells were analysed by Sysmex pre- and post-CD62L selection using the CliniMACS Prodigy. Results shown are from three independent cord blood donors.

FIG. 23. Whole Cord Blood CD62L Selection using the CliniMACS Prodigy. Frequency of lymphocyte subsets were analysed by flow cytometry on cord blood processed CD62L selection. A live gate was set based on the FSC-A/SSC-A profile of the cells and lymphocytes were identified based on the expression of CD45. CD45+ WBC were further anlaysed for expression of CD3 (T cells), CD14 (monocytes) CD56 (NK Cells) and CD20 (B Cells).

FIG. 24. Expansion and Transduction of CD62L selected cells using the CliniMACS Prodigy. The T cell Transduction process on the CliniMACS Prodigy was initiated using cord blood cells that had undergone CD62L selection. The CD62L selected cells were activated with TransAct and transduced with CD19−CAR vector 24 hours later. Cells were expanded for a total of 8 days. (A) WBC count was measured by Sysmex over 8 days of cell expansion in the CliniMACS Prodigy. Results from three independent experiments are shown. (B) At the end of the expansion process the cord blood cells were stained for antibodies against CD3 and CD45 and the T cell purity was analysed by flow cytometry. (C) Transduction efficiency of CD19−CAR was assessed by flow cytometry staining with a α-murine Fab antibody.

FIG. 25: Summary of the three T cell Transduction processes performed using CD62L selected cord blood cells.

FIG. 26: Self-duplicating CRISPR expression cassette generated by incorporation of a pol III promotor and sgRNA sequence into the 3′ LTR of a U3 deleted third generation lentiviral vector.

FIG. 27: Function and effects of Lentiviral terminal-TRAC (TT) guide RNA vectors.

FIG. 28: Transient Cas9 mRNA delivery by electroporation to Terminal-TRAC T cells.

FIG. 29: Scalability of Terminal-TRAC T cell production.

FIG. 30: Characterisation of Terminal-TRAC T cells produced by scaled-up protocol.

FIG. 31: Terminal TRAC-CAR19+TCR− T cells efficiently target CD19+ cells in vitro.

FIGS. 32 to 34: Use of a humanised murine model of leukaemic clearance to assess in vivo function of engineered CAR19 T cells

FIG. 35: Expression of exhaustion marker PD-1 on engineered CAR19 T cells.

FIG. 36: Generation and efficacy of TTCAR20 (TRAC) & TBCAR20 (B2M) peripheral blood T cells

FIG. 37: Scalability of TTCAR20 production

FIG. 38: TT and TB UACT cell manufacture

FIG. 39: Universal TT and TB CAR123 manufacture

FIG. 40—Universal TTCAR22 and TBCAR22 manufacture

FIG. 41—Universal TTCAR20 cord T cell manufacture

FIG. 42—TT-UACT cord T cell manufacture

FIG. 43: multiplexing terminal CRISPR

FIG. 44: TCR devoid rTCR engineered T cells

FIG. 45: Base editing delivered by TTCAR19 vector to disrupt TRAC without DNA cleavage

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural referents unless the content clearly dictates otherwise. Thus, for example, reference to “a molecule” includes “molecules”, reference to “a T-cell” includes two or more such T-cells, reference to “a component” includes two or more such components, and the like.

All publications, patents and patent applications cited herein, whether supra or infra, are hereby incorporated by reference in their entirety.

### Generation of U-ACTs

The disclosure provides a method for generating universal antibody dependent cord T cells (U-ACTs), comprising (a) providing a sample of cord blood; (b) separating cells that express CD62L from the sample, wherein the cells that express CD62L comprise cord blood T cells; (c) introducing into one or more of said cord blood T cells of (b) a nucleic acid sequence encoding an Fc-Receptor (FcR) that comprises (I) an extracellular domain that is capable of binding to a constant domain of an antibody and (II) a transmembrane domain and a cytoplasmic domain that are capable of supporting T cell activation; and (d) disrupting expression of T cell receptor and MHC class I in said cord blood T cells of (c), wherein, in (d), the expression of T cell receptor and/or MHC class 1 is disrupted by (i) introducing one or more CRISPR guide sequences to said cord blood T cells of (c) using a vector that comprises a 3′ long terminal repeat region (LTR) comprising one or more promoter sequences operably linked to the sequence encoding the said CRISPR guide sequence(s); and ii) separately delivering a CRISPR guided DNA modification enzyme to said cord blood T cells of (c) by introducing into them a nucleic acid or protein sequence encoding said CRISPR guided DNA modification enzyme.

According to the method described herein, U-ACTs are generated from cord blood T cells. The use of cord blood T cells is advantageous because they have a naïve phenotype, an immense proliferative potential and potent in vivo activity in transplant recipients. Thus, the method described herein begins with a sample of cord blood. The sample of cord blood may be any type of sample. For instance, the sample of cord blood may be fresh cord blood or frozen cord blood. The sample of cord blood may have been derived from one individual. The sample of cord blood may have been derived from multiple individuals, i.e. a pooled cord blood sample.

Cord blood T cells are obtained from the cord blood sample by separating cells that express CD62L from the sample. Any appropriate method may be used to separate cells that express CD62L from the sample. For instance, the cells that express CD62L may be separated from the sample based on their ability to bind an anti-CD62L antibody. The anti-CD62L antibody may be 145/15 (Miltenyi), DREG-56 (Biolegend, BD), FMC46 (BioRad) or LAM-116 (Merck). Fluorescence activated cell sorting (FACS) or magnetic activated cell sorting (MACS) may be used to separate the cells that express CD62L from the sample. In MACS, magnetic beads are conjugated to the anti-CD62L antibody. Binding of the CD62L-expressing cells to the anti-CD62L antibody therefore tags the cells with magnetic beads. Magnetism can therefore be used to separate the tagged cells from the sample.

The separation step may be manually performed. Alternatively, the separation step may be performed in a system designed for the automated separation of cells. In one aspect, the system is configured for automated production of cord T cells. The system may be a CliniMacs system or a Miltenyi Prodigy system. Other automated cell separation systems are known in the art.

The CD62L-expressing cells separated from the sample comprise cord blood T cells. Thus, the CD62L-expressing cells may comprise CD8+ T cells, or cytotoxic T cells. The CD62L-expressing cells may comprise CD4+ T cells, or helper T cell (T_{H} cells), such as a T_{H}1, T_{H}2, T_{H}3, T_{H}17, T_{H}9, or T_{FH} cells. The CD62L-expressing cells may comprise regulatory T cells (Treg).

In some aspects, the CD62L-expressing cells comprising cord blood T cells are stimulated after separation from the sample of cord blood. For instance, the CD62L-expressing cells may be contacted with an anti-CD3 antibody and/or an anti-CD28 antibody. In this way, the cord blood T cells may be activated or expanded. This can further increase the proportion of cord blood T cells among the selected CD62L-expressing cells. The anti-CD3 antibody and/or the anti-CD28 antibody may be present on microbeads.

Once cord blood T cells are obtained, a nucleic acid sequence that encodes an Fc-Receptor (FcR) is introduced into one or more of the cord blood T cells. An FcR is a protein that is endogenously found on the surface of certain immune cells, such as B lymphocytes, follicular dendritic cells, natural killer cells, macrophages, neutrophils, eosinophils, basophils, human platelets, and mast cells. FcRs are named for their ability to bind to part of an antibody constant region known as the Fc (Fragment, crystallizable) region. FcRs can bind to antibodies that are attached to diseased cells or invading pathogens, stimulating phagocytic or cytotoxic cells to destroy microbes or diseased cells by antibody-mediated phagocytosis or antibody-dependent cell-mediated cytotoxicity respectively.

In the present disclosure invention, the FcR comprises (I) an extracellular domain that is capable of binding to a constant domain of an antibody and (II) a transmembrane domain and a cytoplasmic domain that are capable of supporting T cell activation. The FcR may comprise a CD8 transmembrane domain “stalk” and 4-1BB and CD3ζ cytoplasmic domains. The extracellular domain may comprise a domain derived from antibody light chain. In this case, the FcR has improved dimerization ability, and therefore improved clustering.

The extracellular domain may comprise an extracellular domain of a variant FcRIIIA FcRIIIA is also known as CD16. CD16 is a low affinity FcR, It is naturally found on the surface of natural killer cells, neutrophil polymorphonuclear leukocytes, monocytes and macrophages.

The antibody whose constant domain is bound by the extracellular domain may be an IgG antibody, such as an IgG1 antibody. The antibody may be a monoclonal antibody or a polyclonal antibody. The antibody may be a therapeutic antibody. The antibody may be a human antibody. The antibody may be a humanised antibody. The antibody may be a non-human antibody, such a, canine, equine, bovine, ovine, porcine, murine, feline, leporine, cavine or camelid antibody, having human IgG constant domains. Preferably, the antibody is a therapeutic monoclonal human antibody, or a therapeutic monoclonal humanised antibody.

The antibody may be specific for a marker expressed on a particular type of cells. For instance, the antibody may be specific for a B cell marker, such as CD20. CD20 is an activated-glycosylated phosphoprotein expressed on the surface of all B-cells beginning at the pro-B phase (CD45R+, CD117+) and progressively increasing in concentration until maturity. Preferably, the CD20-specific antibody is Rituximab. Rituximab destroys B cells and is therefore used to treat diseases which are characterized by overactive, dysfunctional, or excessive numbers of B cells. This includes many lymphomas, leukemias, transplant rejection, and autoimmune disorders. The antibody may be Ofatumumab. Ofatumumab may be used to treat chronic lymphocytic leukemia, Follicular non-Hodgkin's lymphoma, Diffuse large B cell lymphoma, rheumatoid arthritis and relapsing remitting multiple sclerosis.

The antibody may be specific for CD22. CD22 is found on the surface of mature B cells and to a lesser extent on some immature B cells. Generally speaking, CD22 is a regulatory molecule that prevents the overactivation of the immune system and the development of autoimmune diseases. Preferably, the CD22-specific antibody is Inotuzumab. Inotuzumab is an anti-cancer drug which may be used to treat non-Hodgkin lymphoma and acute lymphoblastic leukemia.

The antibody may be specific for CD38. CD38 is a glycoprotein found on the surface of many immune cells, including CD4+, CD8+, B lymphocytes and natural killer cells. CD38 also functions in cell adhesion, signal transduction and calcium signaling. Preferably, the CD38-specific antibody is Daratumumab. Daratumumab is an anti-cancer drug targeting multiple myeloma.

The antibody may be specific for CD52. CD52 is a glycoprotein present on the surface of mature lymphocytes, but not on the stem cells from which these lymphocytes were derived. It also is found on monocytes and dendritic cells. Preferably, the CD52-specific antibody is Alemtuzumab. Alemtuzumab is a drug used in the treatment of chronic lymphocytic leukemia (CLL), cutaneous T-cell lymphoma (CTCL), T-cell lymphoma and multiple sclerosis.

The antibody may be specific for EGFR. EGFR is the cell-surface receptor for members of the epidermal growth factor family (EGF family) of extracellular protein ligands. Preferably, the EGFR-specific antibody is Panitumumab. Panitumumab is a drug used in the treatment of colorectal cancer.

The antibody may be specific for Erb2. Erb2 is otherwise known as HER2. HER2 is a member of the human epidermal growth factor receptor (HER/EGFR/ERBB) family. Amplification or over-expression of this oncogene has been shown to play an important role in the development and progression of certain aggressive types of breast cancer. Preferably, the HER2-specific antibody is Herceptin (Trastuzumab) or Pertuzumab. Pertuzumab inhibits the dimerization of HER2 with other HER receptors

The antibody may be specific for CD30. CD30 a cell membrane protein of the tumor necrosis factor receptor family and a tumor marker for lymphoma such as Hodgkin lymphoma (HL) and systemic anaplastic large cell lymphoma (sALCL). Preferably, the CD30-specific antibody is Brentuximab vedotin.

The antibody may be specific for GD2. GD2 is a disialoganglioside expressed on tumors of neuroectodermal origin, including human neuroblastoma and melanoma, with highly restricted expression on normal tissues, principally to the cerebellum and peripheral nerves in humans. Preferably, the GD2-specific antibody is Dinutuximab.

The antibody may be specific for VegfR. VegfR is a receptor for endothelial growth factor (VEGF), an important signaling protein involved in both vasculogenesis (the formation of the circulatory system) and angiogenesis (the growth of blood vessels from pre-existing vasculature). Preferably, the anti-VEGFR antibody is Ramucirumab. By binding to VEGFR2, Ramucirumab works as a receptor antagonist blocking the binding of VEGF to VEGFR2.

The antibody may be specific for a tumour antigen. The antibody may be specific for an antigen associated with an infectious agent, such as a virus, a bacteria or a protozoa.

The cytoplasmic domain of the FcR receptor may comprise an activation domain. The activation domain serves to activate the T cell following engagement of the extracellular domain. For instance, the cytoplasmic domain may comprise one or more of a 41BB activation domain, a CD3 activation domain and a CD3e activation domain. Preferably, the cytoplasmic domain comprises a 41BB activation domain and/or a CD3 activation domain.

The transmembrane domain of the FcR receptor serves to transmit activation signals to the cytoplasmic signal transduction get domains following ligand binding of the extra cellular domains uptown Fc binding. The transmembrane domain may be derived from a molecule other than IgG. Use of a transmembrane domain from a molecule other than IgG avoids problems of antigenicity associated with transmembrane domains derived from IgG. The transmembrane domain may comprise a CD8 activation domain.

The FcR may comprise a spacer. The spacer connects the transmembrane domain to the extracellular domain. The spacer may confer steric effects that influence the strength of activation and inhibition signaling from the target cell and its surface receptors. The spacer may extend to incorporate an immunoglobulin light chain variable region. When an immunoglobulin light chain variable region is used as the spacer, the spacer facilitates FcR dimerization. In turn, dimerization encourages FcRs to cluster on the cell surface, and activation of the T cell via the cytoplasmic activation domains. This results in a stronger signal.

The nucleic acid sequence encoding the FcR may be introduced to the cord blood T cells using any method known in the art. In particular, the cord blood T cells may be transfected or transduced with the nucleic acid sequence.

The term “transduction” may be used to describe virus mediated nucleic acid transfer. A viral vector may be used to transduce the cell with the one or more constructs. Conventional viral based expression systems could include retroviral, lentivirus, adenoviral and adeno-associated (AAV). Non-viral transduction vectors include transposon based systems including PiggyBac and Sleeping Beauty systems. Methods for producing and purifying such vectors are know in the art. The vector is preferably a vector of the invention. The cord blood T cells may be transduced using any method known in the art. Transduction may be in vitro or ex vivo.

The term “transfection” may be used to describe non-virus-mediated nucleic acid transfer. The cord blood T cells may be transfected using any method known in the art. Transfection may be in vitro or ex vivo. Any vector capable of transfecting the cord blood T cells may be used, such as conventional plasmid DNA or RNA transfection. A human artificial chromosome and/or naked RNA may be used to transfect the cell with the nucleic acid sequence or nucleic acid construct. Human artificial chromosomes are described in e.g. Kazuki et al., Mol. Ther. 19(9): 1591-1601 (2011), and Kouprina et al., Expert Opinion on Drug Delivery 11(4): 517-535 (2014). Alternative non-viral delivery systems include DNA plasmids, naked nucleic acid, and nucleic acid complexed with a delivery vehicle, such as a liposome. Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Nanoparticle delivery systems may be used to transfect the cord blood T cells with the nucleic acid sequence or nucleic acid construct. Such delivery systems include, but are not limited to, lipid-based systems, liposomes, micelles, microvesicles and exosomes. With regard to nanoparticles that can deliver RNA, see, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93. Lipid Nanoparticles, Spherical Nucleic Acid (SNA™) constructs, nanoplexes and other nanoparticles (particularly gold nanoparticles) are also contemplated as a means for delivery of a construct or vector in accordance with the invention.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectAmine, fugene and transfectam.

The cord blood T cells may be transfected or transduced under suitable conditions. For instance, the cord blood T cells may be transfected or transduced following activation with combinations of antibodies such as anti-CD3 and anti-CD28 which may be conjugated to beads or polymers and used with or without cytokines such as IL2, IL7, and IL15. The cord blood T cells and agent or vector may, for example, be contacted for between five minutes and ten days, preferably from an hour to five days, more preferably from five hours to two days and even more preferably from twelve hours to one day after activation.

The nucleic acid sequence transduced or transfected into the cord blood T cells gives rise to expression of FcR in the T cells. If the nucleic acid sequence is transduced into the cord blood T cell, the vector used for transduction may comprise a further nucleic acid sequence encoding another molecule useful to the generation of U-ACT. In particular, and as set out below, CRISPR guide sequences targeting a gene associated with TCR or MHC class I expression or other genomic targets may be present in the same vector as the nucleic acid sequence encoding the FcR.

To make the FcR-expressing cord blood T cells universal, their expression of TCR and MHC class I is disrupted. Expression of these molecules may be disrupted using any mechanism known in the art. Exemplary methods included genome editing using zinc finger nucleases (ZFNs), Meganucleases, transcription activator-like effector nucleases (TALENs), or the clustered regularly interspaced short palindromic repeats (CRISPR)/Cas system. The terminal CRISPR approach of the present invention may be used. All of these genome editing methods can disrupt a gene, entirely knocking out all of its output.

Any combination of methods may be used to disrupt TCR and MHC class I expression. For instance, ZFNs may be used to disrupt expression of both molecules. TALENs may be used to disrupt expression of both molecules. CRISPR may be used to disrupt expression of both molecules. Alternatively, ZFNs may be used to disrupt TCR expression and TALENs may be used to disrupt MHC class I expression. ZFNs may be used to disrupt TCR expression and CRISPR may be used to disrupt MHC class I expression. TALENs may be used to disrupt TCR expression and ZFNs may be used to disrupt MHC class I expression. TALENs may be used to disrupt TCR expression and CRISPR may be used to disrupt MHC class I expression. CRISPR may be used to disrupt TCR expression and TALENs may be used to disrupt MHC class I expression. CRISPR may be used to disrupt TCR expression and TALENs may be used to disrupt MHC class I expression. CRISPR in this context refers to conventional CRISPR, or the newly-described terminal CRISPR.

Preferably, at least one of TCR expression and MEW class I expression is disrupted using “terminal CRISPR”. Terminal CRISPR is described in detail below. In brief, terminal CRISPR used to disrupt expression of at least one of TCR expression and MHC class I expression by (i) introducing one or more CRISPR guide sequences to the FcR-expressing cord blood T cells using a vector that comprises a long terminal repeat region (LTR) comprising a H1 and/or a U6 promoter sequence operably linked to the sequence encoding the said CRISPR guide sequence(s); and ii) separately delivering a CRISPR guided DNA modification enzyme to FcR-expressing cord blood T cells of by introducing into them a nucleic acid or protein sequence encoding said CRISPR guided DNA modification enzyme.

Irrespective of the method used to disrupt TCR expression, TCR expression may be disrupted by targeting one or more of the T cell receptor alpha constant (TRAC) locus, TCR beta constant locus, or CD3 receptor complex chains. The TCR beta constant locus may be C1 or C2. Preferably, the TRAC locus is targeted.

Likewise, MHC class 1 may be disrupted by targeting the transporter associated with antigen processing (TAP1 or TAP2) locus, whichever method of disruption is used. The TAP1 locus may be targeted. MEW class 1 may be disrupted by Beta-2 microglobulin (β₂m) locus, whichever method of disruption is used. Preferably, the β₂m locus is targeted. MEW class II molecules may also be disrupted by targeting transcription factors controlling MEW expression such as CIITA, RFX5, RFXAP or RFXANK.

### Generation of TCR− CAR+ T Cells and MEW Class 1-CAR+ T Cells

The invention provides a method for generating T cells that comprise a nucleic sequence encoding a CAR and have disrupted TCR and/or MHC class 1 expression (TCR− CAR+ T cells or MHC1-CAR+ T cells), comprising: (a) providing one or more T cells; (b) introducing into one or more of said T cells of (a) a nucleic acid sequence encoding a CAR; and (c) disrupting expression of TCR and/or MHC class 1 in said T cells (b), wherein, in (c), the expression of TCR and/or MHC class 1 is disrupted by: (i) introducing one or more CRISPR guide sequences to said T cells of (b) using a vector that comprises a 3′ long terminal repeat region (LTR) comprising one or more promoter sequences operably linked to the sequence encoding said CRISPR guide sequence(s); and ii) separately delivering a CRISPR guided DNA modification enzyme to said T cells of (b) by introducing into them a nucleic acid or protein sequence encoding said CRISPR guided DNA modification enzyme.

The CAR may be specific for any antigen, such as CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX. For example, the CAR may be specific for CD19 (i.e. in TCR− CAR19+ T cells), CD20 (i.e. in TCR− CAR20+ T cells), CD22 (i.e. in TCR− CAR20+ T cells) or CD123 (i.e. in TCR− CAR123+ T cells).

TCR− CAR+ T cells or MHC1-CAR+ T cells may be generated from cord blood T cells. Samples of cord blood and methods of separating T cells from a cord blood sample are set out above in relation to the generation of U-ACTs. The advantages of using cord blood T cells in the methods described herein are also set out above.

The T cells of (a) may comprise CD8+ T cells, or cytotoxic T cells. The T cells may comprise CD4+ T cells, or helper T cell (T_{H} cells), such as a T_{H}1, T_{H}2, T_{H}3, T_{H}17, T_{H}9, or T_{FH} cells. The T cells may comprise regulatory T cells (Treg).

The T cells may be stimulated after separation prior to use in (a), for example after separation from a sample of cord blood. For instance, the T cells may be contacted with an anti-CD3 antibody and/or an anti-CD28 antibody. In this way, the T cells may be activated or expanded. The anti-CD3 antibody and/or the anti-CD28 antibody may be present on microbeads. The anti-CD3 antibody and/or the anti-CD28 antibody may be used in combination with cytokines such as interleukin-2, interleukin-7 and interleukin-15, alone or in combination.

Once T cells are provided, a nucleic acid sequence encoding a chimeric antigen receptor (CAR) is introduced into one or more of the T cells. CARs are engineered receptors, which graft an selected specificity onto an immune effector cell. CARs usually incorporate a single chain variable fragment (scfv) derived from the antigen binding regions of an antibody, linked to an intracellular activation domain. Thus, the CAR may comprise an ectodomain capable of binding to an antigen and a transmembrane domain and a cytoplasmic domain that are capable of supporting T cell activation. The ectodomain may comprise an antibody, a monoclonal antibody, or a scfv specific for CD19, for instance. The ectodomain may be specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX. The ectodomain may be specific for CD19, CD20 or CD22. The cytoplasmic domain may comprise one or more of CD3ζ, OX40, CD28 and 4-1BB cytoplasmic domains.

The cytoplasmic domain of the CAR may comprise an activation domain. The activation domain serves to activate the T cell following engagement of the extracellular domain. For instance, the cytoplasmic domain may comprise one or more of a 41BB activation domain, a CD3 activation domain and a CD3e activation domain. Preferably, the cytoplasmic domain comprises a 41BB activation domain and/or a CD3 activation domain.

The nucleic acid sequence encoding the CAR may be introduced to the T cells using any method known in the art. In particular, the T cells may be transfected or transduced with the nucleic acid sequence. Transfection and transduction are described in detail above in relation to the generation of U-ACT.

The nucleic acid sequence transduced or transfected into the T cells gives rise to expression of CAR in the T cells. Preferably, the nucleic acid sequence is transduced into the T cell. In this case, the vector used for transduction may comprise a further nucleic acid sequence encoding another molecule useful to the generation of TCR− CAR+ T cells or MHC1-CAR+ T cells. In particular, and as set out below, CRISPR guide sequences targeting a gene associated with expression of the TCR−CD3 complex and/or a gene associated with the expression of MHC class 1 may be present in the same vector as the nucleic acid sequence encoding the CAR.

To make the TCR− CAR+ T cells more universal by reducing the capability of the cells to cause GVHD in an individual to which they are administered, their expression of TCR is disrupted. MHC expression may also be disrupted, in particular MHC class I. Expression of these molecules may be disrupted using any mechanism known in the art. Exemplary methods included genome editing using zinc finger nucleases (ZFNs), Meganucleases, transcription activator-like effector nucleases (TALENs), or the clustered regularly interspaced short palindromic repeats (CRISPR)/Cas system. The terminal CRISPR approach of the present invention is preferably used. All of these genome editing methods can disrupt a gene, entirely knocking out all of its output.

Any combination of methods may be used to disrupt TCR and MHC class I expression, or TCR and MHC class I expression. For instance, ZFNs may be used to disrupt TCR expression. TALENs may be used to disrupt TCR expression. CRISPR may be used to disrupt TCR expression. ZFNs may be used to disrupt MHC class I expression. TALENs may be used to disrupt MHC class I expression. CRISPR may be used to disrupt MHC class I expression. ZFNs may be used to disrupt expression of both molecules. TALENs may be used to disrupt expression of both molecules. CRISPR may be used to disrupt expression of both molecules. Alternatively, ZFNs may be used to disrupt TCR expression and TALENs may be used to disrupt MHC class I expression. ZFNs may be used to disrupt TCR expression and CRISPR may be used to disrupt MHC class I expression. TALENs may be used to disrupt TCR expression and ZFNs may be used to disrupt MHC class I expression. TALENs may be used to disrupt TCR expression and CRISPR may be used to disrupt MHC class I expression. CRISPR may be used to disrupt TCR expression and TALENs may be used to disrupt MHC class I expression. CRISPR may be used to disrupt TCR expression and TALENs may be used to disrupt MHC class I expression. CRISPR in this context refers to conventional CRISPR, or the terminal CRISPR approach of the present invention.

Preferably, at TCR expression is disrupted using “terminal CRISPR”. MHC class I expression may also be disrupted using terminal CRISPR. Terminal CRISPR is described in detail below.

Irrespective of the method used to disrupt TCR expression, TCR expression may be disrupted by targeting one or more of the T cell receptor alpha constant (TRAC) locus, TCR beta constant locus, or CD3 receptor complex chains. The TCR beta constant locus may be C1 or C2. Preferably, the TRAC locus is targeted.

If terminal CRISPR is used to disrupt TRAC expression in the formation of TCR− CAR+ T cells, the resultant TCR− CAR+ T cells may be referred to as terminal TRAC TCR− CAR+ T cells (TT TCR− CAR+ T cells, e.g. TT TCR− CAR19+ T cells).

Likewise, MHC class 1 may be disrupted by targeting the transporter associated with antigen processing (TAP1 or TAP2) locus, whichever method of disruption is used. The TAP1 locus may be targeted. MHC class 1 may be disrupted by Beta-2 microglobulin (β₂m) locus, whichever method of disruption is used. Preferably, the β₂m locus is targeted. MEW class II molecules may also be disrupted by targeting transcription factors controlling MEW expression such as CIITA, RFX5, RFXAP or RFXANK.

### Generation of Cord Blood T Cells

Umbilical cord blood T cells have unique properties that make them attractive target for cell therapy applications. As set out above, they harbour distinct molecular and cellular characteristics capable of supporting immunotherapeutic effects. They are almost entirely of a naïve phenotype, have extensive proliferative capacity, and can mediate potent anti-viral and anti-leukemic effects in the allogeneic transplant setting. In addition, in contrast to adult donor haematopoietic stem cell transplants, umbilical cord grafts are routinely undertaken with one or more HLA mismatches without notable exacerbations of GVHD or higher rates of rejection. Cord blood donations are collected at birth and usually cryopreserved within 24-48 hours at central processing facilities. To obtain cord blood T cells for therapeutic applications, it is necessary to isolate cord blood T cells from the other cell types present in cord blood, such as haematopoietic stem cells, monocytes, red cells including nucleated red cells and platelets

The present disclosure provides a process to enrich cord blood T cells without using antibodies against their T cell receptor or other key activation ligands. The process allows cord blood T cells to be isolated from umbilical collections that are otherwise difficult to process due high numbers of immature cells including nucleated red cells.

The inventors have found that CD62L is expressed on almost all cord T cells, meaning that cord blood T cells can be isolated from a cord blood sample based on selection for CD62L expression. Using the method described herein, sufficient cord blood T cells can be isolated from a single cord blood donation to engineer enough therapeutic T cells (such as CAR T cells or the U-ACT T cells of the disclosure) for administration to one or more individuals in need thereof. Traditional methods of cord blood T cell isolation, such as density gradient separation (e.g. Ficoll based enrichment), have not yielded sufficient T cells for therapeutic purposes. Thus, the present inventors have advantageously developed a mechanism by which autologous and/or allogeneic cord T cells may be manufactured for therapeutic use.

In accordance with the method of the disclosure, cord blood T cells are obtained from a sample of cord blood. The sample of cord blood may be any type of sample. For instance, the sample of cord blood may be fresh cord blood or frozen cord blood. The sample of cord blood may have been derived from one individual. The sample of cord blood may have been derived from multiple individuals, i.e. a pooled cord blood sample.

Any method may be used to separate cells that express CD62L from the sample. For instance, the cells that express CD62L may be separated from the sample based on their ability to bind an anti-CD62L antibody. The anti-CD62L antibody may be 145/15 (Miltenyi), DREG-56 (Biolegend, BD), FMC46 (BioRad) or LAM-116 (Merck). Fluorescence activated cell sorting (FACS) or magnetic activated cell sorting (MACS) may be used to separate the cells that express CD62L from the sample. In MACS, magnetic beads are conjugated to the anti-CD62L antibody. Binding of the CD62L-expressing cells to the anti-CD62L antibody therefore tags the cells with magnetic beads. Magnetism can therefore be used to separate the tagged cells from the sample.

The separation step may be manually performed. Alternatively, the separation step may be performed in a system designed for the automated separation of cells. In one aspect, the system is configured for automated production of cord T cells. The system may be a CliniMacs system, or a Miltenyi Prodigy system. Other automated cell separation systems are known in the art.

The CD62L-expressing cells separated from the sample comprise cord blood T cells. Thus, the CD62L-expressing cells may comprise CD8+ T cells, or cytotoxic T cells. The CD62L-expressing cells may comprise CD4+ T cells, or helper T cell (T_{H} cells), such as a T_{H}1, T_{H}2, T_{H}3, T_{H}17, T_{H}9, or T_{FH} cell. The CD62L-expressing cells may comprise regulatory T cells (Treg).

In some aspects, the CD62L-expressing cells comprising cord blood T cells are stimulated after separation from the sample of cord blood. For instance, the CD62L-expressing cells may be contacted with an anti-CD3 antibody and/or an anti-CD28 antibody. In this way, the cord blood T cells may be activated or expanded. This can further increase the proportion of cord blood T cells among the selected CD62L-expressing cells. The anti-CD3 antibody and/or the anti-CD28 antibody may be present on microbeads.

The CD62L-expressing cells are amenable to stimulation with anti-CD3 and/or anti-CD28 antibodies, because their TCR (CD3) and CD28 co-receptor has not been “touched” during selection. The only molecule that has been touched is CD62L, which is bound by anti-CD62L antibody. Thus, activation and expansion of cord blood T cells obtained by the method of the invention is improved relative to activation and expansion of cord blood T cells obtained by traditional methods, such as density gradient separation.

After separation from the sample of cord blood, or after subsequent activation and expansion with anti-CD3 and/or anti-CD28 antibodies, a molecule useful for therapeutic purposes may be introduced to the cord blood T cells. For instance, an FcR of the disclosure or a CAR may be introduced to the cord blood T cells. The CAR may be specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX. The cord blood T cells may be transfected or transduced with a nucleic acid sequence encoding the molecule to give expression of the molecule in the cord blood T cells. Transfection and transduction are described in detail above.

### Terminal CRISPR

Gene editing approaches based on Zinc Finger Nucleases and TALENs have recently reached clinical phase applications, with T cells the first ex vivo engineered cells to be applied in man. Efficiency of editing has been modest, with delivery of mRNA encoding nuclease reagents by electroporation. Emerging CRISPR/Cas9 reagents are opening new possibilities of genome editing, but a key hurdle remains efficient delivery in primary human cells. In particular, one limitation is the ability to scale the RNA or protein delivery.

Lentiviral mediated delivery of CRISPR guide sequences and Cas9 CRISPR nuclease has been reported. Such vectors integrate and stably express target specific guide RNA using polIII promoter elements, and separately express Cas9 protein under the control of internal mammalian/viral promoter elements for gene editing effects. These lentiviral vectors are suitable for experimental purposes in a research setting, but stable expression of Cas9 would be problematic for therapeutic use. There would be ongoing Cas9 complexing with CRISPR RNA, resulting in further DNA scission effects, possibly including off-target activity. In addition Cas9 is of bacterial origin and could trigger immunogenic responses.

The present inventors have developed a new CRISPR technique, “terminal CRISPR”, that addresses the problems associated with the traditional lentiviral mediated delivery of CRISPR guide sequences and Cas9 CRISPR nuclease. Terminal CRISPR is an integrating self-inactivating vector, designed to deliver and stably express therapeutic transgene(s) (such as chimeric antigen receptors (CARs), recombinant TCR, suicide gene, antiviral restriction factor, recombinant coding DNA for inherited gene defects, or an FcR of the invention) under the control of an internal human promoter and to simultaneously mediate highly specific DNA scission through expression of CRISPR guide nucleic acids. The guide nucleic acids act in concert with CRISPR guided DNA modification enzyme delivered separately to the target cell, for instance by mRNA electroporation. The CRISPR guide sequences and associated promoters are incorporated into a 3′ terminal repeat (LTR) sequence of the vector plasmid, and are thereby duplicated during reverse transcription. For instance, if the CRISPR guide sequences are incorporated in the 3′LTR, they are copied to the 5′LTR during reverse transcription.

The configuration of the terminal CRISPR vector has a number of advantages:

i. Avoiding promoter interference during vector genome expression during vector manufacture or with transgene expression, thereby retaining titre and expression comparable to conventional vectors;

ii. Doubling guide RNA expression through duplication effects; and

iii. Linking and thereby restricting guide effects to cells transduced with vector and expressing transgene.

iv. Linking transgene expression to guide effects, thereby facilitating purification and enrichment of transduced cells.

Furthermore, in contrast with more traditional genome editing techniques, such as ZFNs or TALENs, the CRISPR approach in general has several further advantages:

i. A greater number of gene loci can be targeted than with TALENs, ZFNs, Mega-talens or meganucleases;

ii. Multiplex effects can be more easily secured; and

iii. Greatly reduced cost of manufacture of vector plus a single batch of Cas9 mRNA compared to multiple (pairs) of targeting nuclease reagents.

CRISPR/Cas9 gene disruption is conventionally mediated by DNA double-strand breaks (DSBs). CRISPR base editing inactivates genes by converting four codons CAA, CAG, CGA, and TGG into STOP codons (Billon et al, Molecular Cell, Volume 67, Issue 6, 21 Sep. 2017, Pages 1068-1079; Kuscu et al Nature Methods 14, 710-712 2017). CRISPR base editing has the advantage of not causing DSBs, and thus reduces the risk of translocations. This is especially true in the multiplex setting. CRISPR guides can be designed to specifically target a splice acceptor/donor consensus sequences at an exon termini. This is exemplified in the Examples below, in which targeting the splice donor site in TRAC exon 1 results in retention of intron sequences resulting in abnormal TCRa protein production, leading to disruption of TCRab expression without the creation of DNA breaks. A similar approach could be used to disrupt normal RNA expression for genes across the genome and adds to the toolbox of targeting the four codons above.

To arrive at the terminal CRISPR platform, expression of a CRISPR guided DNA modification enzyme has been divorced from the vector delivering the CRISPR guide sequences. Instead, a CRISPR guided DNA modification enzyme is delivered separately to the target cell. For instance, a CRISPR guided DNA modification enzyme may be provided as CRISPR nuclease mRNA and delivered by electroporation. A CRISPR guided DNA modification enzyme may be provided as a protein. Separate delivery of a CRISPR guided DNA modification enzyme allows the CRISPR guided DNA modification enzymeto be expressed transiently and to have time-limited effects, as it becomes diluted in rapidly dividing cells. A CRISPR guided DNA modification enzyme provided transiently is also less likely to be immunogenic.

Appropriate CRISPR guided DNA modification enzyme are known in the art. The guided DNA modification enzyme may be a CRISPR nuclease. The CRISPR nuclease may be Cas. Preferably, the CRISPR nuclease is Cas9. The Cas9 may be Streptococcus pyogenes Cas9 (SpCas9) or Staphylococcus aureus Cas9 (SaCas9). CRISPR nucleases from any bacteria may though be used. Dead Cas or nickases could also be used, to give rise to effects such as repression or cytidine deamination. The CRISPR guided DNA modification enzyme may be a cytidine deaminase. The CRISPR guided DNA modification enzyme may be repressor or activator CRISPR guided DNA modification enzyme.

In terminal CRISPR, expression of CRISPR guide sequences is mediated by promoters contained in a 3′long terminal repeat region (LTR) present in the vector. During reverse transcription, the LTR region is duplicated and becomes incorporated into both the 5′ and 3′ LTR, resulting in two expression cassettes. Thus, guide sequence expression is increased, and the likelihood of and interference effect between CRISPR guide sequences and any transgene additionally encoded in the vector is reduced.

As set out in the Examples, terminal CRISPR vectors that also encode a transgene, such as a CAR, have been found to be highly effective, with numerous beneficial effects. In particular:
No significant reduction in vector titre or transgene expression as a result of additions to the LTRs in the terminal CRISPR vectors has been observed. This means that the invention is readily translatable to large scale applications.
The Terminal-CRISPR approach removes the cost of bespoke mRNA production, and only requires a single stock of Cas9 mRNA. There are already platforms aiming to gene edit CAR expressing T cells, including TALENs, that are in clinical phase evaluation. These platforms rely on mRNA delivery of two specific TALEN pairs for each locus targeted, adding notably to the cost of each manipulation. This cost is avoided with the terminal CRISPR approach.
The ability to include CRISPR guide sequences in the vector that also encodes a transgene, such as CAR or a FcR of the invention, ensures that knock out effects can only occur in transduced cells. This improves safety and reduces the risk of unwanted effects
LTR duplication results is replication of CRISPR sequences, and thereby supports enhanced expression.

Accordingly, the invention provides a method for delivering CRISPR guide sequences and CRISPR guided DNA modification enzyme to a cell, comprising (a) introducing one or more CRISPR guide sequences to said cell using a vector that comprises a 3′ long terminal repeat region (LTR) comprising one or more promoter sequences operably linked to the sequence encoding the said CRISPR guide sequence(s); and (b) separately delivering CRISPR guided DNA modification enzyme to said cell of (a) by introducing into it a nucleic acid or protein sequence encoding said CRISPR guided DNA modification enzyme.

The vector may be a viral vector. The vector may be a lentiviral vector. The vector may be a 3^{rd} generation lentiviral vector. The vector may be a gamma retroviral vectors and an alpha retroviral vector.

The LTR may comprise a H1 promoter. The LTR may comprise a U6 promoter. Each promoter (H1, U6 or otherwise) may be operably linked to a sequence encoding one CRISPR guide sequence. The LTR may comprise two or more different promoter sequences. For example, the LTR may comprise a H1 promoter sequence and a U6 promoter sequence, and optionally one or more other different promoter sequences.

The LTR may comprise several different promoters each operably linked to a sequence encoding one CRISPR guide sequence. In other words, the LTR may comprise two or more sequences encoding a CRISPR guide sequence each operably linked to a different promoter sequence. The promoter sequence to which each of the two or more sequences is operably linked is a different type of promoter sequence. For instance, a first sequence encoding a CRISPR guide sequence may be operably linked to a U6 promoter sequence, while a second sequence encoding a CRISPR guide sequence may be operably linked to a H1 promoter sequence. The H1 promoter sequence may be a full length or minimal H1 Pol III promoter sequence.

When the LTR comprises several promoters each operably linked to a sequence encoding one CRISPR guide sequence, the CRISPR guide sequences encoded by the sequences operably linked to each promoter may be the same or different. That is, when the LTR comprises two or more sequences encoding a CRISPR guide sequence each operably linked to a promoter sequence, the CRISPR guide sequences encoded by each of the two sequences may be the same or different. Preferably, the sequences are different. If the CRISPR guide sequences are different, they may target the same locus or different loci. Targeting different loci allows the expression of two or more different target molecules to be disrupted using the same terminal CRISPR vector, i.e. the terminal CRISPR approach can be “multiplexed”. Any combination of target molecules may be targeted in this way using a single terminal CRISPR vector. For instance, a single terminal CRISPR vector may be use to target (i) TRAC and CD52, (ii) TRAC and PD1, (iii) PD1 and β2M, (iv) TRAC and CD123, or (v) TRAC and CD52. Operably linking each sequence encoding a different CRISPR guide sequence to a different promoter sequence prevents recombination effects, allowing each guide sequence to be efficiently expressed.

Following delivery of the vector, the promoter sequence(s) may be duplicated during reverse transcription such that it becomes incorporated into both the 5′ and 3′ LTRs. Likewise, the guide sequence(s) may be duplicated during reverse transcription such that it becomes incorporated into both the 5′ and 3′ LTRs.

The nucleic acid sequence encoding the CRISPR guided DNA modification enzyme may be RNA, such as mRNA. The nucleic acid sequence encoding the CRISPR guided DNA modification enzyme may be DNA.

The vector may further comprise a sequence encoding a CAR. The CAR may be specific for CD10, CD19, CD20, CD22, CD30 CD33, CD123, CD45 erb-B2, CEA, IL13R, Ror, kappa light chain, TCR-beta constant 1, TCR-beta constant 2, MAGE-A1, MUC1, PSMA, VEGF-R, Her2, or CAIX. The CAR may be specific for CD19. The vector may further comprise a sequence encoding an FcR of the invention.

One or more of the CRISPR guide sequences may be specific for the TRAC locus. One or more of the CRISPR guide sequences may be specific for the TAP1 locus. One or more of the CRISPR guide sequences may be specific for the TAP2, Beta-2 microglobulin (β₂m), CIITA, RFX5, RFXAP or RFXANK locus. One or more or more of the CRISPR guide sequences may be specific for a locus controlling a checkpoint inhibitor pathway. One or more of the CRISPR guide sequences is specific for the locus controlling expression of CD52. One or more of the CRISPR guide sequences is specific for a locus controlling the expression of an antigen targeted by a CAR, chimeric FcR or monoclonal antibody expressed by the cells.

In one aspect, the vector comprises a sequence encoding a CAR specific for CD19, and one or more CRISPR guide sequences specific for a locus controlling the expression of the TCR−CD3 complex. The vector may comprise a sequence encoding a CAR specific for CD19, and one or more CRISPR guide sequences specific for the TRAC locus.

As set out above, this terminal CRISPR method for delivering CRISPR guide sequence and CRISPR guided DNA modification enzyme to a cell may be used to disrupt the expression of TCR and/or MHC class I in T cells. For TCR disruption, the guide sequence(s) may be specific for the TRAC locus, TCR beta constant locus or CD3 locus. For MHC class I disruption, the guide sequence(s) may be specific for the TAP1, TAP2, β₂m, CIITA, RFX5, RFXAP or RFXANK locus. The terminal CRISPR method may be used in the generation of U-ACTs of the invention. In this case, the nucleic acid sequence encoding the FcR and the CRISPR guide sequence(s) may be introduced to the cord blood T cells in the same vector. The terminal CRISPR method may be used in the generation of TCR− CAR19+ T cells of the invention. In this case, the nucleic acid sequence encoding the CAR specific for CD19 and the CRISPR guide sequence(s) specific for a locus controlling the expression of the TCR−CD3 complex may be introduced to the T cells in the same vector. Delivery in the same vector is associated with the advantages set out above.

The terminal CRISPR method for delivering CRISPR guide sequence and CRISPR guided DNA modification enzyme to a cell further has almost limitless applications in cell engineering. Terminal CRISPR may be used to modify any type of cell or therapeutic cell. For instance, terminal CRISPR may be used in a cord blood T cell, a peripheral blood lymphocyte, a hematopoietic stem cell, a mesenchymal stem cell, a fibroblast, or a keratinocyte. The cell modified using terminal CRISPR may be autologous or allogeneic to an individual into which the cell is to be administered. Terminal CRISPR may be used to disrupt the expression of any gene expressed in any cell type. The terminal CRISPR vector may be used to introduce any transgene into the any cell. Exemplary uses of terminal CRISPR are as follows.

Terminal CRISPR may be used to modify a cord blood T cell. The cord blood T cell may be obtained using the method of the invention. When terminal CRISPR is used to modify a cord blood T cell, it may be used disrupt expression of TCR and/or MHC class I. Concurrently, the terminal CRISPR may be used to introduce a FcR and/or a CAR. For instance, terminal CRISPR may be used to (i) disrupt expression of TCR and MHC class I and introduce a FcR such as the FcR of the invention; (ii) disrupt expression of TCR and MHC class I and introduce a CAR, such as a CAR specific for CD19, CD20, CD22, CD33, CD123 or CD3; or (iii) disrupt expression of TCR or MHC1 and introduce a CAR specific for CD3.

Likewise, terminal CRISPR may be used to modify an allogeneic peripheral blood lymphocyte (PBL), such as a T cell or a B cell. When terminal CRISPR is used to modify an allogeneic PBL, it may be used disrupt expression of TCR and/or MHC class I. Concurrently, the terminal CRISPR may be used to introduce a FcR and/or a CAR. For instance, terminal CRISPR may be used to (i) disrupt expression of TCR and MHC class I and introduce a FcR such as the FcR of the invention; (ii) disrupt expression of TCR and MEW class I and introduce a CAR, such as a CAR specific for CD19, CD20, CD22, CD33, CD123 or CD3; or (iii) disrupt expression of TCR or MHC1 and introduce a CAR specific for CD3.

Terminal CRISPR may also be used to modify an autologous cell, such as an autologous PBL or hematopoietic stem cell (HSC). Using terminal CRISPR, and autologous PBL or HSC may be modified in several key ways:
i. disruption of TCR and/or CD3 expression and introduction of a recombinant TCR (rTCR). This approach leads to enhanced expression of rTCR through removal of competition for CD3, and prevention of cross pairing, reducing the risk of autoreactive TCRs.
ii. disruption of a viral co-receptor and introduction of an anti-viral factor such as a restriction factor. For instance, expression of CCR5 (an HIV co-receptor) may be disrupted. The restriction factor TRIM5CypA, C46 HIV fusion inhibitor, TRIM21, CylcophilinA, APOBEC, SAMHD1 or Tetherin may be targeted.
iii. disruption of gain-of function mutations and introduction of replacement cDNA. This therapeutic approach allows a two-prong attack using a single vector. Gain of function mutations include mutations in genes such as STAT1, STAT3, NFKB1A, CARD11, CXCR4 and PI3K.
iv. disruption of transgene-silencing pathways and introduction of a protein lacking or mutated cell. This gene therapy approach release inhibition on transgene expression, allowing sustained, longer term expression of replacement protein. Human silencing Hub (HUSH) complex pathways, or TASOR (transgene activator suppressor) protein may be targeted. MMP8 or Periphilin may be targeted.
v. disruption of checkpoint inhibitor pathways (e.g. PD-1) and introduction of a suicide gene. Disruption of checkpoint inhibitor pathways unleashes an immune system attack on cancer cells. Introduction of a suicide gene provides an “off switch” for the disinhibited cells. Any suitable suicide gene may be used. Suicide genes are well-know in the art and include Herpes simplex virus thymidine kinase and mutated variants, inducible caspases and cell surface proteins incorporating epitope targets for antibodies against CD20 or EGFR.

It can therefore be appreciated that the potential utility of the Terminal CRISPR approach is very broad, and is not limited to U-ACTs of the invention, or indeed to T cells, immune system cells or indeed therapeutic cells at all.

FcR

The disclosure provides an FcR that comprises (I) an extracellular domain that is capable of binding to a constant domain of an antibody and (II) a transmembrane domain and a cytoplasmic domain that are capable of supporting T cell activation. The FcR may comprise a CD8 transmembrane domain “stalk” and 4-1BB and CD3 cytoplasmic domains. The extracellular domain may comprise a domain derived from antibody light chain. In this case, the FcR has improved dimerization ability with improved clustering and favourable steric properties.

The extracellular domain may comprise an extracellular domain of a variant FcRIIIA. FcRIIIA is also known as CD16. CD16 is a low affinity FcR, It is naturally found on the surface of natural killer cells, neutrophil polymorphonuclear leukocytes, monocytes and macrophages.

The antibody whose constant domain is bound by the extracellular domain may be an IgG antibody, such as an IgG1 antibody. The antibody may be a monoclonal antibody or a polyclonal antibody. The antibody may be a therapeutic antibody. The antibody may be a human antibody. The antibody may be a humanised antibody. The antibody may be a non-human antibody, such as canine, equine, bovine, ovine, porcine, murine, feline, leporine, cavine or camelid antibody, having human IgG constant domains. Preferably, the antibody is a therapeutic monoclonal human antibody, or a therapeutic monoclonal humanised antibody.

The antibody may be specific for a marker expressed on a particular type of cells. For instance, the antibody may be specific for a B cell marker, such as CD20. Preferably, the antibody is Rituximab. The antibody may be specific for a tumour antigen. The antibody may be specific for an antigen associated with an infectious agent, such as a virus, a bacteria or a protozoa.

Other preferred antibodies described above in relation to U-ACTs also relate to FcRs of the disclosure.

The cytoplasmic domain of the FcR receptor may comprise an activation domain. The activation domain serves to activate the T cell following engagement of the extracellular domain. For instance, the cytoplasmic domain may comprise one or more of a 41BB activation domain, a CD3 activation domain or a CD3e activation domain. Preferably, the cytoplasmic domain comprises a 41BB activation domain and/or a CD3 activation domain.

The transmembrane domain of the FcR receptor serves to transmit activation signals to the cytoplasmic signal transduction get domains following ligand binding of the extra cellular domains uptown Fc binding. The transmembrane domain may comprise a CD8 activation domain.

The FcR may comprise a spacer. The spacer connects the transmembrane domain to the extracellular ligand binding domain, and provides steric function as set out above. The spacer may be an immunoglobulin light chain variable region. When an immunoglobulin light chain variable region is used as the spacer, the spacer facilitates FcR dimerization. In turn, dimerization encourages activation of the T cell via the cytoplasmic activation domains.

The present disclosure also provides a dimer of the FcR of the disclosure.

### Nucleic Acid Sequence

The present disclosure provides a nucleic acid sequence encoding an FcR of the invention. The nucleic acid construct may comprise DNA and/or RNA. The nucleic acid construct may be double stranded or single stranded. For instance, the nucleic acid construct may comprise dsDNA or ssDNA. The nucleic acid construct may comprise dsRNA and/or ssRNA.

### Vectors of the Invention

The present invention provides a vector that may be used for terminal CRISPR as described above. The vector comprises a LTR comprising one or more promoter sequences operably linked to a sequence encoding one or more CRISPR guide sequences.

The LTR may comprise a H1 promoter. The LTR may comprise a U6 promoter. Each promoter (H1, U6 or otherwise) may be operably linked to a sequence encoding one CRISPR guide sequence. The LTR may comprise two or more different promoter sequences. For example, the LTR may comprise a H1 promoter sequence and a U6 promoter sequence, and optionally one or more other different promoter sequences.

The LTR may comprise several different promoters each operably linked to a sequence encoding one CRISPR guide sequence. In other words, the LTR may comprise two or more sequences encoding a CRISPR guide sequence each operably linked to a different promoter sequence. The promoter sequence to which each of the two or more sequences is operably linked is a different type of promoter sequence. For instance, a first sequence encoding a CRISPR guide sequence may be operably linked to a U6 promoter sequence, while a second sequence encoding a CRISPR guide sequence may be operably linked to a H1 promoter sequence. The H1 promoter sequence may be a full length or minimal H1 Pol III promoter sequence.

When the LTR comprises several promoters each operably linked to a sequence encoding one CRISPR guide sequence, the CRISPR guide sequences encoded by the sequences operably linked to each promoter may be the same or different. That is, when the LTR comprises two or more sequences encoding a CRISPR guide sequence each operably linked to a promoter sequence, the CRISPR guide sequences encoded by each of the two sequences may be the same or different. Preferably, the sequences are different. If the CRISPR guide sequences are different, they may target the same locus or different loci. Targeting different loci allows the expression of two or more different target molecules to be disrupted using the same terminal CRISPR vector, i.e. the terminal CRISPR approach can be “multiplexed”. Any combination of target molecules may be targeted in this way using a single terminal CRISPR vector. For instance, a single terminal CRISPR vector may be use to target (i) TRAC and CD52, (ii) TRAC and PD1, (iii) PD1 and β2M, (iv) TRAC and CD123, or (v) TRAC and CD52. Operably linking each sequence encoding a different CRISPR guide sequence to a different promoter sequence prevents recombination effects, allowing each guide sequence to be efficiently expressed.

The vector may comprise a nucleic acid sequence encoding an FcR of the invention. The vector may comprise a nucleic acid sequence encoding a CAR, such as a CAR specific for CD10, CD19, CD20, CD22, CD30 CD33, CD123, CD45 erb-B2, CEA, IL13R, Ror, kappa light chain, TCR-beta constant 1, TCR-beta constant 2, MAGE-A1, MUC1, PSMA, VEGF-R, Her2, or CAIX. The CAR may be specific for CD19. The vector may comprise a nucleic acid sequence encoding a rTCR. The vector may comprise a nucleic acid sequence encoding an anti-viral molecule, such as a restriction factor. Restriction factors are known in the art, such as TRIM5CypA C46 HIV fusion inhibitor, TRIM21CylcophilinA, APOBEC, SAMHD1 or Tetherin. The vector may comprise a nucleic acid sequence encoding a suicide gene. Suicide genes are known in the art.

The sequence encoding one or more CRISPR guide sequences may be capable of a disrupting expression of a gain-of-function mutant allele of a gene. In this case, the vector may encode a normal variant of a gene. In this way, the unwanted gain-of-function mutant allele can be replaced with a normally-functioning gene. Optionally, gene is STAT1, STAT3, CXCR4, NFKB1A, CARD11, CARD15, STING, NLRP3, NLRC4, PSTPIP1, PIK3CD or PIK3R1.

The CRISPR guide sequence may be specific for a locus controlling the expression of the TCR−CD3 complex. For instance, the CRISPR guide sequence may be specific for the TRAC locus, TCR beta constant locus or CD3 locus. The CRISPR guide sequence may be specific for a locus controlling the expression of MHC class I. For instance, the CRISPR guide sequence may be specific for the TAP1, TAP2, CIITA, RFX5, RFXAP or RFXANK or β₂m locus. The CRISPR guide sequence may be specific for a locus controlling a checkpoint inhibitor pathway. The CRISPR guide sequence may be specific for a locus associated with a gain of function mutation, such as mutations in genes such as STAT1, STAT3, NFKB1A, CARD11, CXCR4 and PI3K. The CRISPR guide sequence may be specific for a locus controlling transgene silencing pathway. For instance, Human silencing Hub (HUSH) complex pathways including TASOR (transgene activator suppressor) protein may be targeted.

In one aspect, the vector comprises a nucleic acid sequence encoding a CAR, and one or more of the CRISPR guide sequences are specific for a locus controlling the expression of the TCR−CD3 complex. One or more of the CRISPR guide sequences may be specific for the TRAC locus. The CAR may be specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX.

The vector may contain any number of CRISPR promoter sequences and guide sequences. The vector may contain any number of nucleic acid sequences encoding a transgene, such as those mentioned above. Any combination of promoter sequences, guide sequences and transgene-encoding nucleic acid sequences may be used. The exact combination of these components contained in the vector will be determined by the anticipated application for the vector. Exemplary applications are set out above under the terminal CRISPR heading. The vector may contain any combination of components necessary to achieve the particular aim of each application

The present disclosure also provides a vector comprising the nucleic acid sequence encoding an FcR of the disclosure, and a cell comprising said vector.

In any case, the vector may be a viral vector. Preferably, the viral vector is a lentivirus, a retrovirus, an adenovirus, an adeno-associated virus (AAV), a vaccinia virus or a herpes simplex virus. Methods for producing and purifying such vectors are know in the art. Preferably, the viral vector is a gamma-retrovirus or a lentivirus. The lentivirus may be a modified HIV virus suitable for use in delivering genes. The lentivirus may be a Simian Immunodeficiency Virus (SIV), Feline Immunodeficiency Virus (Hy), or equine infectious anemia virus (EQIA) based vector. The viral vector may comprise a targeting molecule to ensure efficient transduction with the nucleic acid sequence or nucleic acid construct. The targeting molecule will typically be provided wholly or partly on the surface of the viral vector in order for the molecule to be able to target the virus to T-cells. The viral vector is preferably replication deficient.

The vector may be a non-viral vector. Preferably, the non-viral vector is a DNA plasmid, a naked nucleic acid, a nucleic acid complexed with a delivery vehicle, or an artificial virion. The non-viral vector may be a human artificial chromosome. When the non-viral vector is a nucleic acid complexed with a delivery vehicle, the delivery vehicle may be a liposome, virosome, or immunoliposome. Integration of a plasmid vector may be facilitated by a transposase such as sleeping beauty or PiggyBAC.

### U-ACTs

The disclosure provides universal antibody dependent cord T cell (U-ACT) that comprises a FcR of the disclosure invention and has disrupted T cell receptor and MHC class I expression. The U-ACT of the disclosure may be produced using any of the methods of the invention.

The U-ACT may be any type of T-cell. The U-ACT may be a CD4+ T-cell, or helper T-cell (T_{H} cell), such as a T_{H}1, T_{H}2, T_{H}3, T_{H}17, T_{H}9, or T_{FH} cell. The U-ACT may be a regulatory T-cell (Treg). The U-ACT is preferably a CD8+ T-cell, or cytotoxic T-cell.

The U-ACT may comprise one or more, such as two or more, three or more, four or more, five or more or ten or more, nucleic acid sequences encoding FcR of the disclosure. When the U-ACT comprises two or more sequences encoding a FcR of the disclosure, the sequences may encode the same FcR or different FcRs.

The U-ACT may comprise one or more, such as two or more, three or more, four or more, five or more or ten or more, nucleic acid sequences encoding a CAR. When the U-ACT comprises two or more sequences encoding a CAR, the sequences may encode the same CAR or different CARs. Thus, the U-ACT may be a CAR T cell.

The U-ACT may have reduced or completely eliminated expression of TCR. The U-ACT may have reduced or completely eliminated expression of one or more genes associated with expression of the TCR−CD3 complex. The U-ACT may lack one or more genes associated with expression of the TCR−CD3 complex. That is, one or more genes associated with expression of the TCR−CD3 complex may be deleted in the U-ACT.

Accordingly, the U-ACT may have a reduced or completely eliminated capacity to induce GVHD following administration to a HLA-mismatched recipient or patient.

The U-ACT may have reduced or completely eliminated expression of MHC class I and/or MHC class II. The U-ACT may lack one or more genes associated with expression of MHC class I and/or MHC class II. Accordingly, the U-ACT may be subject to minimal amount of rejection when administered to a HLA-mismatched recipient or patient.

TCR− CAR T cells

The invention provides a T cell that comprises a nucleic sequence encoding a CAR and has disrupted TCR expression (a TCR− CAR+ T cell). The TCR− CAR+ T cell of the invention may be produced using any of the methods of the invention.

The CAR of the TCR− CAR+ T cell may be specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1 MUC1, PSMA, VEGF-R, Her2, or CAIX. For instance, the CAR may be specific for CD19, CD20, CD22 or CD123, to give a TCR− CAR19+, TCR− CAR20+, TCR− CAR22+ or TCR− CAR123 cell respectively.

The TCR− CAR+ T cell may be any type of T-cell. The TCR− CAR+ T cell may be a CD4+ T-cell, or helper T-cell (T_{H} cell), such as a T_{H}1, T_{H}2, T_{H}3, T_{H}17, T_{H}9, or T_{FH} cell. The TCR− CAR+ T cell may be a regulatory T-cell (Treg). The TCR− CAR+ T cell is preferably a CD8+ T-cell, or cytotoxic T-cell.

The TCR− CAR+ T cell may comprise one or more, such as two or more, three or more, four or more, five or more or ten or more, nucleic acid sequences encoding a CAR. When the TCR− CAR+ T cell comprises two or more sequences encoding a CAR, the sequences may encode the same CAR or different CARs.

The TCR− CAR+ T cell may comprise one or more, such as two or more, three or more, four or more, five or more or ten or more, nucleic acid sequences encoding FcR of the invention. When the TCR− CAR+ T cell comprises two or more sequences encoding a FcR of the disclosure, the sequences may encode the same FcR or different FcRs. Thus, the TCR− CAR19+ T cell may be a U-ACT.

The TCR− CAR+ T cell may have reduced or completely eliminated expression of TCR. The TCR− CAR+ T cell may have reduced or completely eliminated expression of one or more genes associated with expression of the TCR−CD3 complex. That is, one or more genes associated with expression of the TCR−CD3 complex may be deleted in the TCR− CAR+ T cell. The TCR− CAR+ T cell may lack one or more genes associated with expression of the TCR−CD3 complex. The TCR− CAR+ T cell may have reduced or completely eliminated expression of TRAC. The TCR− CAR+ T cell may lack the TRAC gene. The TRAC gene may be deleted in the TCR− CAR19+ T cell. Accordingly, the TCR− CAR+ T cell may have a reduced or completely eliminated capacity to induce GVHD following administration to a HLA-mismatched recipient or patient.

The TCR− CAR+ T cell may have reduced or completely eliminated expression of MEW class I and/or MEW class II. The TCR− CAR+ T cell may lack one or more genes associated with expression of MHC class I and/or MEW class II. Accordingly, the TCR− CAR+ T cell may be subject to minimal amount of rejection when administered to a HLA-mismatched recipient or patient.

### MHC1-CAR T Cells

The invention provides a T cell that comprises a nucleic sequence encoding a CAR and has disrupted MHC class 1 expression (a MHC1-CAR+ T cell). The MHC1-CAR+ T cell of the invention may be produced using any of the methods of the invention.

The CAR of the MHC1-CAR+ T cell may be specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX. For instance, the CAR may be specific for CD19, CD20, CD22 or CD123, to give a MHC1-CAR19+, MHC1-CAR20+, MHC1-CAR22+ or MHC1-CAR123 cell respectively.

The MHC1-CAR+ T cell may be any type of T-cell. The MHC1-CAR+ T cell may be a CD4+ T-cell, or helper T-cell (T_{H} cell), such as a T_{H}1, T_{H}2, T_{H}3, T_{H}17, T_{H}9, or T_{FH} cell. The MHC1-CAR+ T cell may be a regulatory T-cell (Treg). The MHC1-CAR+ T cell is preferably a CD8+ T-cell, or cytotoxic T-cell.

The MHC1-CAR+ T cell may comprise one or more, such as two or more, three or more, four or more, five or more or ten or more, nucleic acid sequences encoding a CAR. When the MHC1-CAR+ T cell comprises two or more sequences encoding a CAR, the sequences may encode the same CAR or different CARs.

The MHC1-CAR+ T cell may comprise one or more, such as two or more, three or more, four or more, five or more or ten or more, nucleic acid sequences encoding FcR of the invention. When the MHC1-CAR+ T cell comprises two or more sequences encoding a FcR of the disclosure, the sequences may encode the same FcR or different FcRs. Thus, the MHC1-CAR19+ T cell may be a U-ACT.

The MHC1-CAR+ T cell may have reduced or completely eliminated expression of MHC1. The MHC1-CAR+ T cell may have reduced or completely eliminated expression of one or more genes associated with expression of WIC class 1. That is, one or more genes associated with expression of WIC class 1 may be deleted in the MHC1-CAR+ T cell. The MHC1-CAR+ T cell may lack one or more genes associated with expression of WIC class 1. The MHC1-CAR+ T cell may have reduced or completely eliminated expression of β2M. The MHC1-CAR+ T cell may lack the β2M gene. The β2M gene may be deleted in the MHC1-CAR+ T cell. Accordingly, the MHC1-CAR+ T cell may be subject to minimal amount of rejection when administered to a patient.

The MHC1-CAR+ T cell may have reduced or completely eliminated expression of TCR and/or MHC class II. The MHC1-CAR+ T cell may lack one or more genes associated with expression of the TCR−CD3 complex and/or MHC class II. Accordingly, the MHC1-CAR+ T cell may have a reduced or completely eliminated capacity to induce GVHD following administration to a patient.

### Medical Uses

The disclosure provides a U-ACT of the disclosure for use in a method of treatment of the human or animal body. The disclosure also provides a U-ACT of the invention for use in a method of treating a neoplastic condition, and autoimmune condition, an infectious condition, an inflammatory condition or a haematological disorder.

The invention further provides a TCR− CAR+ T cell of the invention for use in a method of treatment of the human or animal body. The invention also provides a TCR− CAR+ T cell of the invention for use in a method of treating a neoplastic condition, and autoimmune condition, an infectious condition, an inflammatory condition or a haematological disorder.

The invention further provides a MHC1-CAR+ T cell of the invention for use in a method of treatment of the human or animal body. The invention also provides a MHC1-CAR+ T cell of the invention for use in a method of treating a neoplastic condition, and autoimmune condition, an infectious condition, an inflammatory condition or a haematological disorder.

The disclosure additionally provides:
a U-ACT of the disclosure for use in the manufacture of a medicament for the treatment of the human or animal body;
a U-ACT of the disclosure for use in the manufacture of a medicament for the treatment of a neoplastic condition, and autoimmune condition, an infectious condition, an inflammatory condition or a haematological disorder;
a TCR− CAR+ T cell of the invention for use in the manufacture of a medicament for the treatment of the human or animal body;
a TCR− CAR+ T cell of the invention for use in the manufacture of a medicament for the treatment of a neoplastic condition, and autoimmune condition, an infectious condition, an inflammatory condition or a haematological disorder;
a MHC1-CAR+ T cell of the invention for use in the manufacture of a medicament for the treatment of the human or animal body;
a MHC1-CAR+ T cell of the invention for use in the manufacture of a medicament for the treatment of a neoplastic condition, and autoimmune condition, an infectious condition, an inflammatory condition or a haematological disorder.

The neoplastic condition is preferably cancer. The cancer may be anal cancer, bile duct cancer (cholangiocarcinoma), bladder cancer, blood cancer, bone cancer, bowel cancer, brain tumours, breast cancer, colorectal cancer, cervical cancer, endocrine tumours, eye cancer (such as ocular melanoma), fallopian tube cancer, gall bladder cancer, head and/or neck cancer, Kaposi's sarcoma, kidney cancer, larynx cancer, leukaemia, liver cancer, lung cancer, lymph node cancer, lymphoma, melanoma, mesothelioma, myeloma, neuroendocrine tumours, ovarian cancer, oesophageal cancer, pancreatic cancer, penis cancer, primary peritoneal cancer, prostate cancer, Pseudomyxoma peritonei, skin cancer, small bowel cancer, soft tissue sarcoma, spinal cord tumours, stomach cancer, testicular cancer, thymus cancer, thyroid cancer, trachea cancer, unknown primary cancer, vagina cancer, vulva cancer or endometrial cancer. The leukaemia is preferably acute lymphoblastic leukaemia, acute myeloid leukaemia (AML), chronic lymphocytic leukaemia or chronic myeloid leukaemia. The lymphoma may be Hodgkin lymphoma or non-Hodgkin lymphoma. The cancer may be primary cancer or secondary cancer.

The autoimmune condition may be alopecia areata, autoimmune encephalomyelitis, autoimmune hemolytic anemia, autoimmune hepatitis, dermatomyositis, diabetes (type 1), autoimmune juvenile idiopathic arthritis, glomerulonephritis, Graves' disease, Guillain-Barré syndrome, idiopathic thrombocytopenic purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, uveitis or vitiligo.

The inflammatory condition may be an allergic disorder, such as atopic dermatitis, allergic airway inflammation or perennial allergic rhinitis.

The infectious condition may be a bacterial, viral, fungal, protozoal or other parasitic infection.

The haematological disorder may be Acute lymphoblastic leukemia (ALL); Acute myeloid leukemia (AML) (or the subtype acute promyelocytic leukemia, APL); Amyloidosis; Anemia; Aplastic anemia; Bone marrow failure syndromes; Chronic lymphocytic leukemia (CLL); Chronic myeloid leukemia (CML); Deep vein thrombosis (DVT); Diamond-Blackfan anemia; Dyskeratosis congenita (DKC); Eosinophilic disorders; Essential thrombocythemia; Fanconi anemia; Gaucher disease; Hemochromatosis; Hemolytic anemia; Hemophilia; Hereditary spherocytosis; Hodgkin's lymphoma; Idiopathic thrombocytopenic purpura (ITP); Inherited bone marrow failure syndromes; Iron-deficiency anemia; Langerhans cell histiocytosis; Large granular lymphocytic (LGL) leukemia; Leukemia; Leukopenia; Mastocytosis; Monoclonal gammopathy; Multiple myeloma; Myelodysplastic syndromes (MDS); Myelofibrosis; Myeloproliferative neoplasms (MPN); Non-Hodgkin's lymphoma; Paroxysmal nocturnal hemoglobinuria (PNH); Pernicious anemia (B12 deficiency); Polycythemia vera; Porphyria; Post-transplant lymphoproliferative disorder (PTLD); Pulmonary embolism (PE); Shwachman-Diamond syndrome (SDS); Sickle cell disease; Thalassemias; Thrombocytopenia; Thrombotic thrombocytopenic purpura (TTP); Venous thromboembolism; Von Willebrand disease; Waldenstrom's macroglobulinemia (lymphoplasmacytic lymphoma).

The disclosure further provides a universal antibody dependent cord T cell (U-ACT) for use in a method of depleting immune cells and/or bone marrow cells in an individual. The individual may be a patient preparing for a transplant. The transplant may be from an allogeneic or HLA-mismatched (or partially mismatched) donor. The transplant may be of an organ, a tissue, or cells. The method of depleting immune cells and/or bone marrow cells may be performed prior to transplantation of the organ, tissue or cells into the individual. In this way, the individual is “conditioned” prior to receiving the transplant. By targeting the U-ACT against host immune cells and/or bone marrow compartments, host immunity is depleted. Thus, the subsequent transplant is less likely to be rejected.

The disclosure further provides TCR− CAR19+ T cells, TCR− CAR20+ T cells, TCR− CAR22+ T cells, MHC1-CAR19+ T cells, MHC1-CAR20+ T cells or MHC1− CAR22+ T cells) for use in a method of depleting immune cells and/or bone marrow cells in an individual. The individual may be a patient preparing for a transplant. The transplant may be from an allogeneic or HLA-mismatched (or partially mismatched) donor. The transplant may be of an organ, a tissue, or cells. The method of depleting immune cells and/or bone marrow cells may be performed prior to transplantation of the organ, tissue or cells into the individual. In this way, the individual is “conditioned” prior to receiving the transplant. By targeting the U-ACT against host immune cells and/or bone marrow compartments, host immunity is depleted. Thus, the subsequent transplant is less likely to be rejected.

The invention further provides TCR− CAR19+ T cells, TCR− CAR20+ T cells, TCR− CAR22+ T cells, MHC1-CAR19+ T cells, MHC1-CAR20+ T cells or MHC1-CAR22+ T cells for use in a method of depleting B cells. The method may be carried out in an individual. The individual may be a patient preparing for a transplant. The transplant may be from an allogeneic or HLA-mismatched (or partially mismatched) donor. The transplant may be of an organ, a tissue, or cells. The method of depleting B cells may be performed prior to transplantation of the organ, tissue or cells into the individual. In this way, the individual is “conditioned” prior to receiving the transplant. By depleting B cells, host immunity is depleted. Thus, the subsequent transplant is less likely to be rejected.

Due to their B cell depleting capabilities, the TCR− CAR19+ T cells, TCR− CAR20+ T cells, TCR− CAR22+ T cells, MHC1-CAR19+ T cells, MHC1-CAR20+ T cells or MHC1-CAR22+ T cells of the invention may be used in a method of treating an infection (such as Epstein Barr Virus (EBV) infection) or autoimmunity. Alternatively, the B cells depleted may be malignant and the TCR− CAR19+ T cells, TCR− CAR20+ T cells, TCR− CAR22+ T cells, MHC1-CAR19+ T cells, MHC1-CAR20+ T cells or MHC1-CAR22+ T cells may be used in a method of treating a tumour or a cancer.

The disclosure further provides a method of treating a neoplastic condition, an autoimmune condition, an infectious condition, a haematological disorder, or an inflammatory condition in a patient in need thereof, the method comprising administering to the patient an effective number of U-ACTs of the disclosure. The invention also provides a method of treating a neoplastic condition, an autoimmune condition, an infectious condition, a haematological disorder, or an inflammatory condition in a patient in need thereof, the method comprising administering to the patient an effective number of TCR− CAR+ T cells or MHC1-CAR+ T cells of the invention. The method may further comprise administering to the patient a therapeutic antibody. The antibody may be administered in the same composition as the U-ACTs, MHC1-CAR+ T cells or TCR− CAR+ T cells. The antibody may be administered separately from the U-ACTs, MHC1-CAR+ T cells or TCR− CAR+ T cells. If the antibody and the U-ACTs, MHC1-CAR+ T cells or TCR− CAR+ T cells are administered separately, (i) the antibody may be administered before the U-ACTs, MHC1-CAR+ T cells or TCR− CAR+ T cells, (ii) the antibody and the U-ACTs, MHC1-CAR+ T cells or TCR− CAR+ T cells may be administered concurrently, or (iii) the antibody may be administered after the U-ACTs, MHC1-CAR+ T cells or TCR− CAR+ T cells. The antibody may be an antibody that is capable of being bound by one or more of the FcRs expressed by the U-ACTs administered to the patient. The neoplastic condition may be B cell cancer. The antibody may be Rituximab.

### Pharmaceutical Compositions

The U-ACTs of the disclosure may be provided as a pharmaceutical composition. Likewise, he TCR− CAR+ T cells or MHC1-CAR+ T cells of the invention may be provided as a pharmaceutical composition. The pharmaceutical composition preferably comprises a pharmaceutically acceptable carrier or diluent. The pharmaceutical composition may be formulated using any suitable method. Formulation of cells with standard pharmaceutically acceptable carriers and/or excipients may be carried out using routine methods in the pharmaceutical art. The exact nature of a formulation will depend upon several factors including the cells to be administered and the desired route of administration. For example, the formulation may comprise isotonic phosphate buffered saline with EDTA with 7.5% DMSO and 4% human albumin serum. Cells may be cryopreserved using a controlled rate freezer stored in the vapour phase of liquid nitrogen until required. Cells may be thawed at the bedside in a waterbath and infused into a vein over a period of 5 minutes. Suitable types of formulation are fully described in Remington's Pharmaceutical Sciences, 19th Edition, Mack Publishing Company, Eastern Pennsylvania, USA.

The U-ACTs, MHC1-CAR+ T cells, TCR− CAR+ T cells or pharmaceutical composition may be administered by any route. Suitable routes include, but are not limited to, intravenous, intramuscular, intraperitoneal or other appropriate administration routes. The U-ACTs, MHC1-CAR+ T cells, TCR− CAR+ T cells or pharmaceutical composition are preferably administered intravenously.

Compositions may be prepared together with a physiologically acceptable carrier or diluent. Typically, such compositions are prepared as liquid suspensions of cells. The cells may be mixed with an excipient which is pharmaceutically acceptable and compatible with the active ingredient. Suitable excipients are, for example, water, saline, dextrose, glycerol, of the like and combinations thereof.

In addition, if desired, the pharmaceutical compositions of the invention may contain minor amounts of auxiliary substances such as wetting or emulsifying agents, pH buffering agents, and/or adjuvants which enhance effectiveness. The composition preferably comprises human serum albumin.

One suitable carrier or diluents is Plasma-Lyte A®. This is a sterile, nonpyrogenic isotonic solution for intravenous administration. Each 100 mL contains 526 mg of Sodium Chloride, USP (NaCl); 502 mg of Sodium Gluconate (C6H11NaO7); 368 mg of Sodium Acetate Trihydrate, USP (C2H3NaO2.3H2O); 37 mg of Potassium Chloride, USP (KCl); and 30 mg of Magnesium Chloride, USP (MgCl2.6H2O). It contains no antimicrobial agents. The pH is adjusted with sodium hydroxide. The pH is 7.4 (6.5 to 8.0).

The U-ACTs, MHC1-CAR+ T cells or TCR− CAR+ T cells are administered in a manner compatible with the dosage formulation and in such amount will be therapeutically effective. The quantity to be administered depends on the subject to be treated, the disease to be treated, and the capacity of the subject's immune system. Precise amounts of U-ACTs, MHC1-CAR+ T cells or TCR− CAR+ T cells required to be administered may depend on the judgement of the practitioner and may be peculiar to each subject.

Any suitable number of U-ACTs, MHC1-CAR+ T cells or TCR− CAR+ T cells may be administered to a subject. For example, at least, or about, 0.2×10⁶, 0.25×10⁶, 0.5×10⁶, 1.5×10⁶, 4.0×10⁶ or 5.0×106 cells per kg of patient may administered. For example, at least, or about, 10⁵, 10⁶, 10⁷, 10⁸, 10⁹ cells may be administered. As a guide, the number of cells to be administered may be from 10⁵ to 10⁹, preferably from 10⁶ to 10⁸. Typically, up to 2×10⁸ U-ACTs, MHC1-CAR+ T cells or TCR− CAR19+ T cells are administered to an adult patient and 2-5×10⁶ to an infant. In such cases where cells are administered or present, culture medium may be present to facilitate the survival of the cells. In some cases the cells of the invention may be provided in frozen aliquots and substances such as DMSO may be present to facilitate survival during freezing. Such frozen cells will typically be thawed and then placed in a buffer or medium either for maintenance or for administration.

The following Examples illustrate the present disclosure.

### EXAMPLES

### Example 1—Terminal CRISPR

We have demonstrated highly efficient gene editing effects using the terminal CRISPR configuration in cells that are first transduced with vector and then electroporated with Cas9 mRNA.

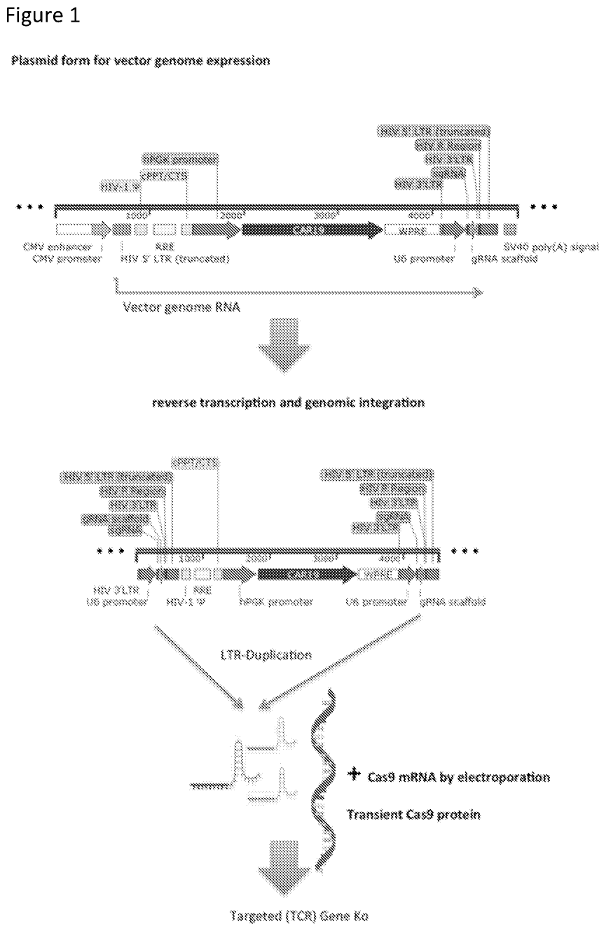

FIG. 1 provides a schematic representation of the “Terminal CRISPR” lentiviral plasmid. The vector is a third generation, integration competent but replication incompetent, self-inactivating lentivirus derived from HIV-1, with deleted U3 regions in the 3′LTR. This configuration requires accessory factors from three other packaging plasmids in order to produce functional virions. Expression of a therapeutic transgene is driven by an internal promoter (in this example PGK) and the vector incorporates a HIV central polypurine tract (CPPT) for nuclear entry and a mutated woodchuck postregulatory element (WPRE) for increased gene expression and titre. Virions delivery vector genome in RNA form, which undergoes reverse transcription an genomic integration. Incorporation of one or more CRISPR RNA coding DNA cassettes into the 3′LTR under the control of an RNA promotor (such as U6 or H1) allows stable expression of CRISPR RNA when the viral vector undergoes reverse transcription and integration as proviral DNA. During this process, elements within the deleted U3 region duplicate and transpose to the 5′LTR. In the absence of Cas9, there are no DNA scission effects, but when Cas9 is provided by transient transfection or electroporation, CRISPR guide is available for complex formation and targeted DNA modification. The figure highlights the example of CRISPR/Cas9 derived from Streptococcus pyognes, and could be adapted for other species including Staphylococcus aureus derived Cas9. In addition, modified Cas9 enzymes such as nickases and deactivated versions linked to other gene modification elements such as cytidine deaminase or activator and repressor domains could be enjoined in this system.

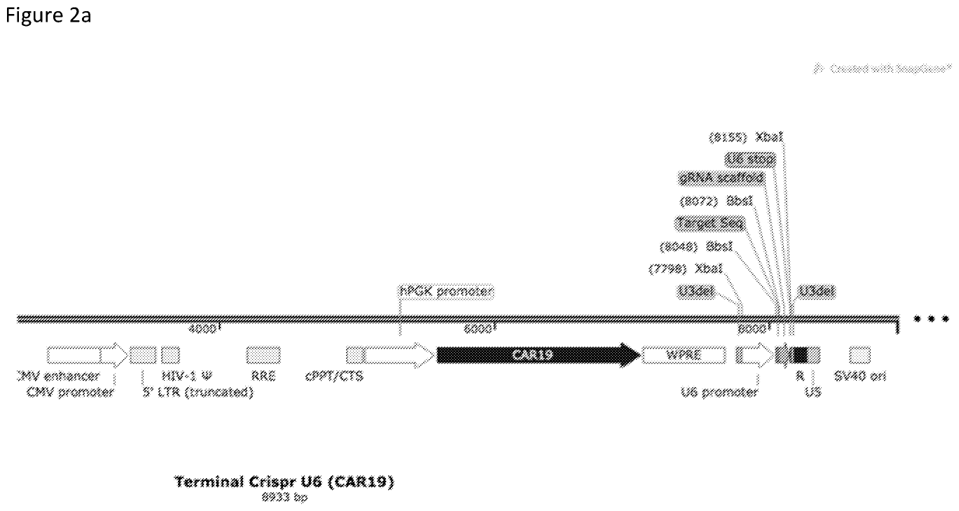

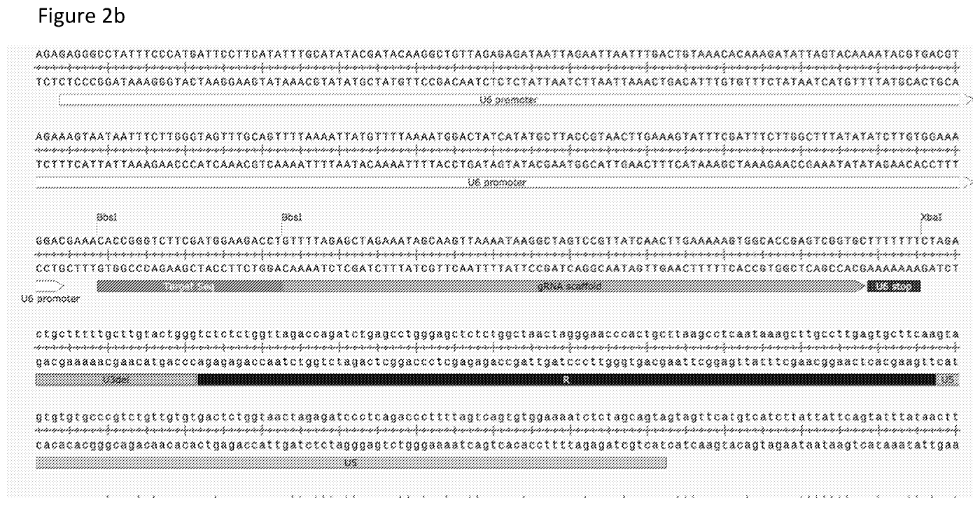

FIG. 2 shows the design of Terminal CRISPR long terminal repeat. Incorporation of a U6 promoter and CRISPR guide cassette into the deleted U3 region of the 3′HIV LTR, flanked by Xba1 sites to facilitate substitution with H1 or other cassettes. Bsb1 sites have been introduced to allow target site sequences to be readily removed and substituted. The scaffold elements and U6 stop are included within a cassette that is sited proximal to the repeat (R) region to ensure duplication and transposition to the 5′ LTR during reverse transcription. The U5 region is retained intact.

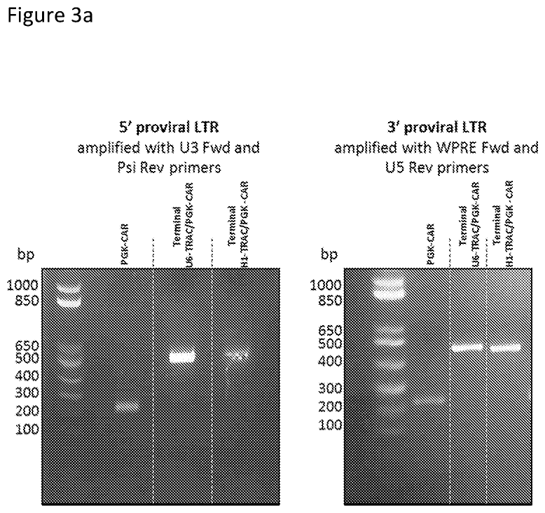

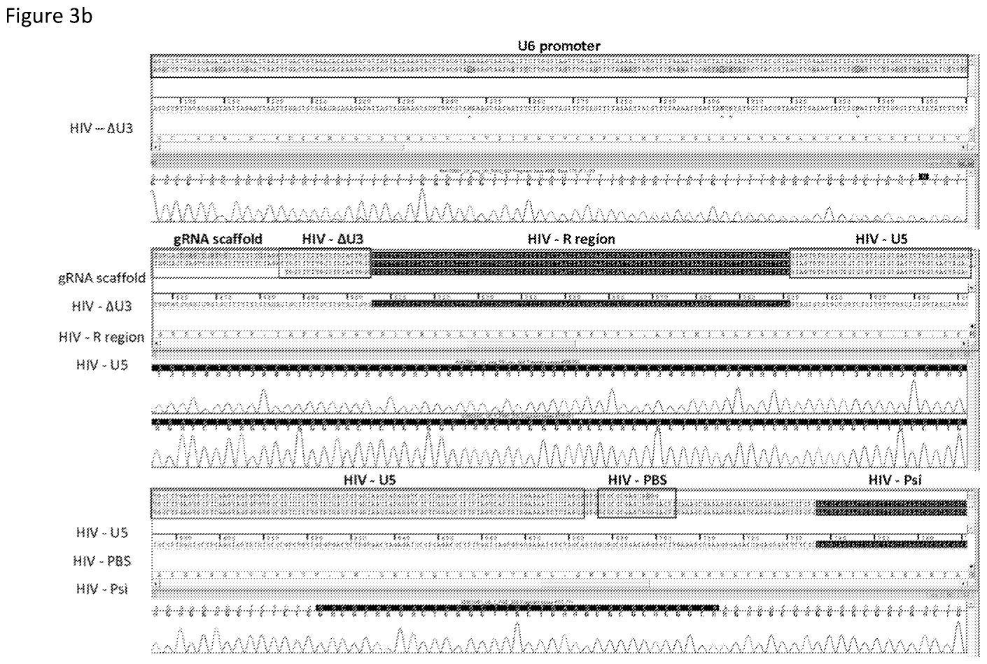

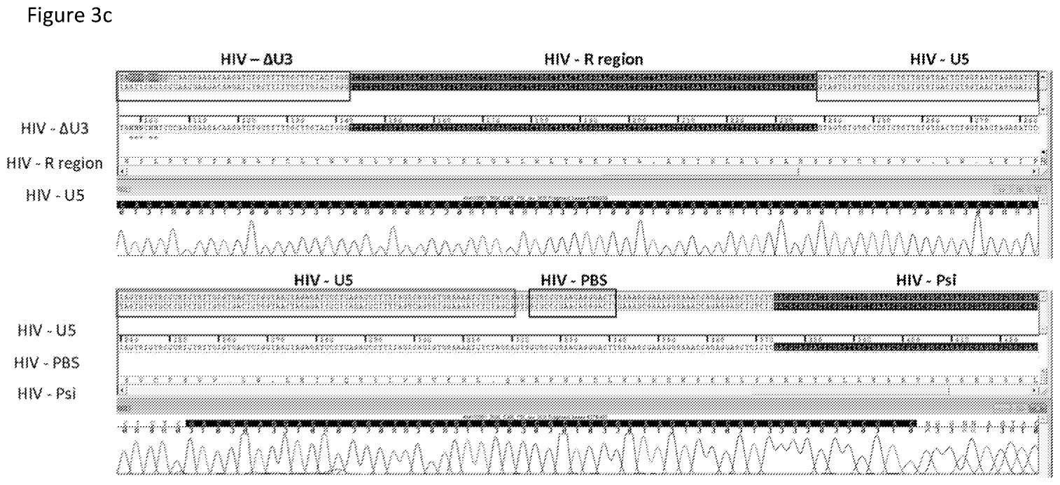

The present inventors have verified duplication of the terminal CRISPR elements (FIG. 3). Specifically, FIG. 3a shows Gel electrophoresis of DNA generated by PCR of genomic DNA from transduced primary T cells using primers targeting the 5′ (U3 Fwd and Psi rev primers) and 3′ (WPRE Fwd and U5 Rev primers) proviral integrated LTRs. Comparison is drawn with a conventional PGK-CAR lentiviral vector and Terminal U6-TRAC/PGK-CAR and Terminal H1-TRAC/PGK-CAR which both yielded larger sized bands. FIG. 3b. Sequencing of 5′ LTR confirmed CRISPR duplication events in the in Terminal Crispr transduced cells. For comparison the usual 5′LTR proviral sequence after conventional sin vector transduction is shown in FIG. 3c.

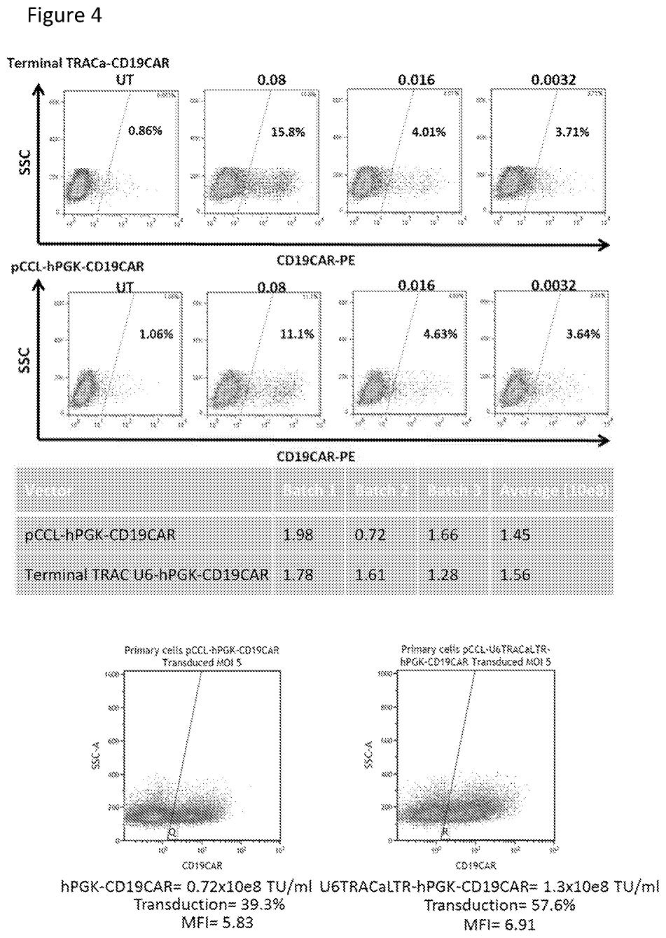

The terminal CRISPR configuration was demonstrated to maintain titre and transgene expression (FIG. 4). Inclusion of ectopic sequences, especially expression cassettes including promoter elements and stop signals, into lentiviral configurations can impair vector titre and interfere with transgene expression. The Terminal Crispr configuration reduces the risk of these effects by incorporating elements within a carefully defined region of the 3′LTR. Titre of Terminal TRAC-CD19CAR viral vector alongside conventional hPGK-CD19CAR was assessed by flow cytometry of a defined number of 293T cells exposed to serial dilution (volumes shown in ul) of vector. CD19CAR expression was detected using an anti-Fab antibody. Three batches of terminal vector all reached target titres above 10⁸/ml after ultracentrifugation, confirming the scalability of the terminal CRISPR vectors. Furthermore, these vector support high level transduction of primary T cells and there was no difference in the intensity of transgene expression in primary cells transduced with each vector at an MOI of 5 (lower panel).

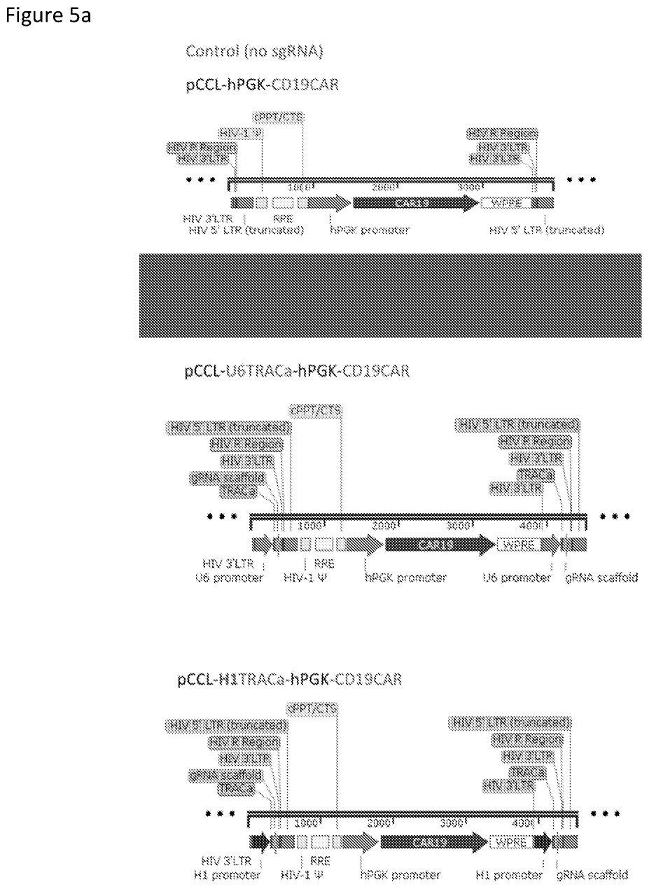

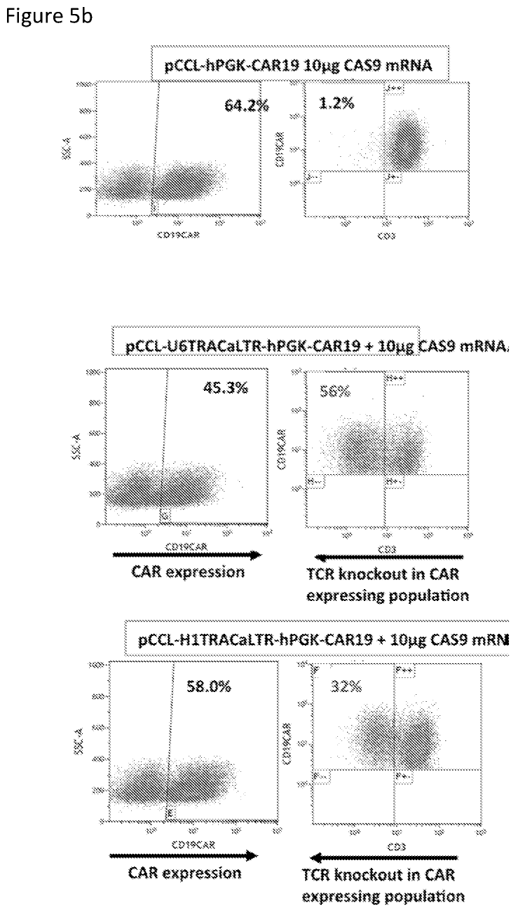

T cell receptor knockout using Terminal CRISPR vectors expressing hPGK-CAR19 was demonstrated (FIG. 5). Primary human T cells were activated by anti-CD3/CD28 stimulation and exposed to a single round of lentiviral transduction at MOIS using a conventional vector in comparison to terminal crispr versions incorporating U6 or H1 driven CRISPR sequences specific for the TRAC locus. Cas9 mRNA (10 ug, Trilink, USA) was delivered by electroporation and flow cytometry for CAR expression undertaken using anti-Fab staining. Reduction in TCR expression of 45% and 58% exceeded target aspirations of 20%, with no reduction with the control vector.

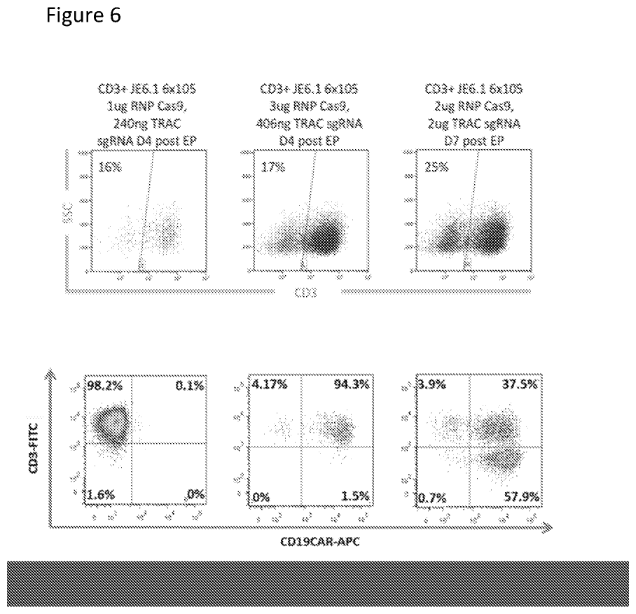

FIG. 6 shows comparison data using Ribonucleoprotein delivery. Specifically, the upper panels show knockout effects in Jurkat T cells using ribonucleoprotein delivery by electroporation of TRAC specific sgRNA complexed with Cas9 protein and Terminal TRAC-CD19CAR vector. Titration of TRAC specific guide RNA and Cas9 RNP in JE6.1 Jurkats (6×10⁵ CD3+TCR+) and measurement of CD3−(TCR−) populations by flow cytometry over time. The lower panels show an example of the terminal U6 TRAC vector in combination with 3 ug Cas9 mRNA electroporation in Jurkat T cells for comparison.

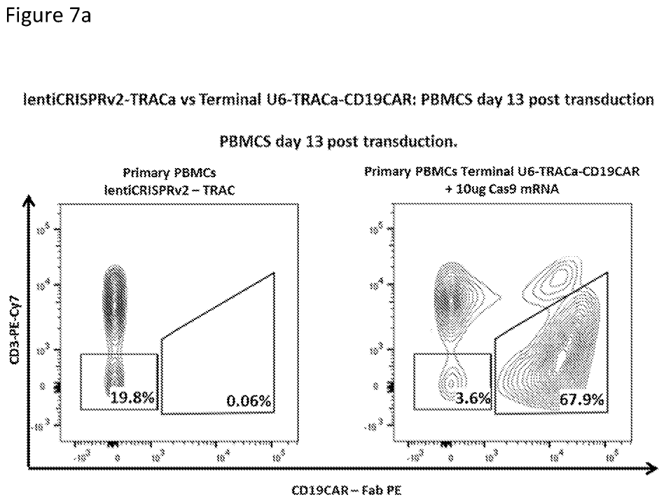

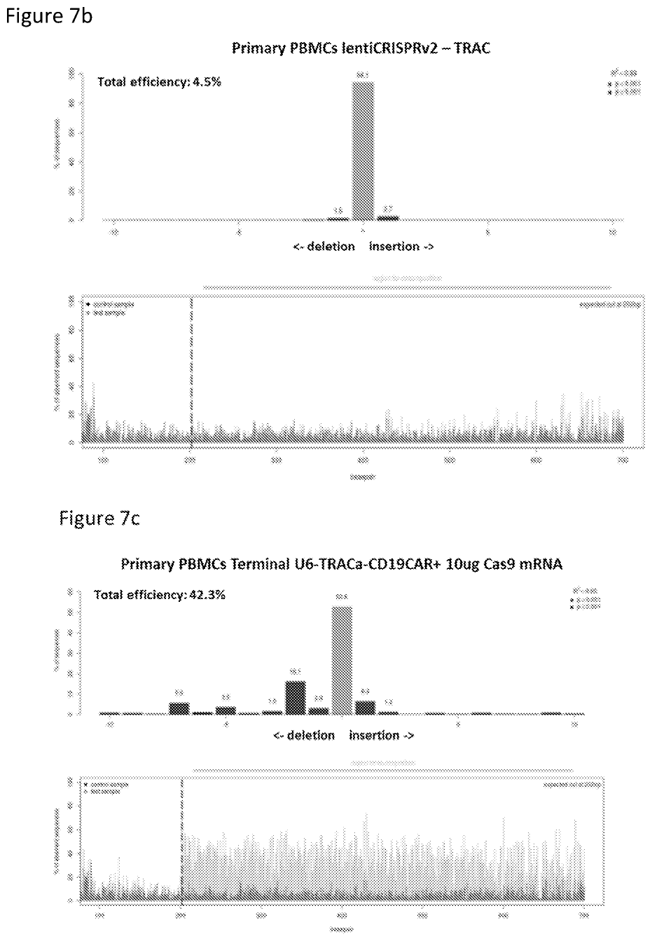

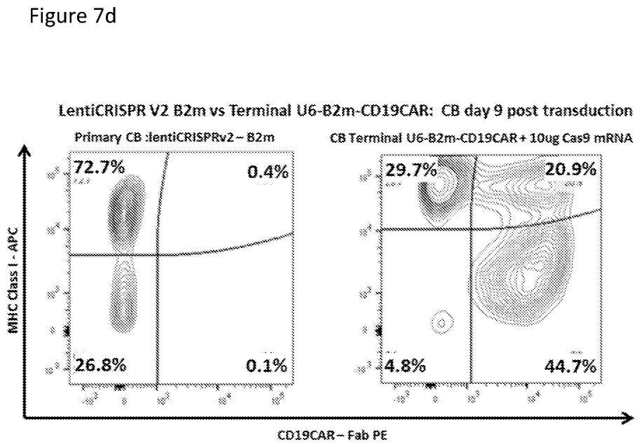

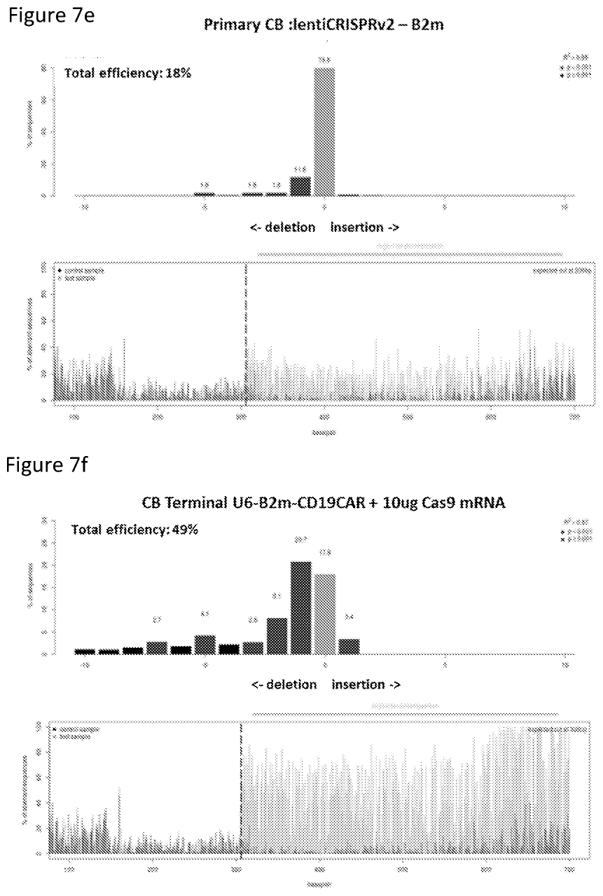

FIG. 7 shows comparison data using alternative lentiviral-CRISPR/Cas9 vectors and terminalU6 β₂m CRISPR vector. Integration competent lentiviral vectors encoding both CRISPR guide against β₂M and a Cas9 gene (lentiCRISPRv2 from Zhang labs) were used to transduce primary T cells or cord blood T cells after activation with anti-CD3/CD28. In FIGS. 7a and 7b knockout of TCR/CD3 was approximately 20% by flow cytometry and 4.5% by TIDE genomic analysis as described (Brinkman et al, Nucl. Acids Res. (2014)). In comparison, PBMCs in FIGS. 7a and 7c transduced with the Terminal U6-TRAC-CD19CAR vector and electroporated with 10 ug of Cas9 mRNA (Trilink, US) showed a CD3/TCR knockout of 68% by flow cytometry and 43% by TIDE analysis. Note that allelic exclusion operates at the TRAC locus. In FIGS. 7d and 7e knockout of MEW class I of around 26% is shown by flow cytometry and 19% by TIDE analysis. In comparison, cord T cells transduced with terminal U6-β₂m CD19CAR expressing vector and electroporated with 10 ug Cas9 mRNA (Trilink, US), were over 44% MEW I negative by flow cytometry and 49% by TIDE analysis. Note in the lentiCRISPRv2 vector system, ongoing Cas9 and guide expression is anticipated, whereas, no ongoing Cas9 effects are expected when delivered by mRNA in conjunction with the Terminal vectors.

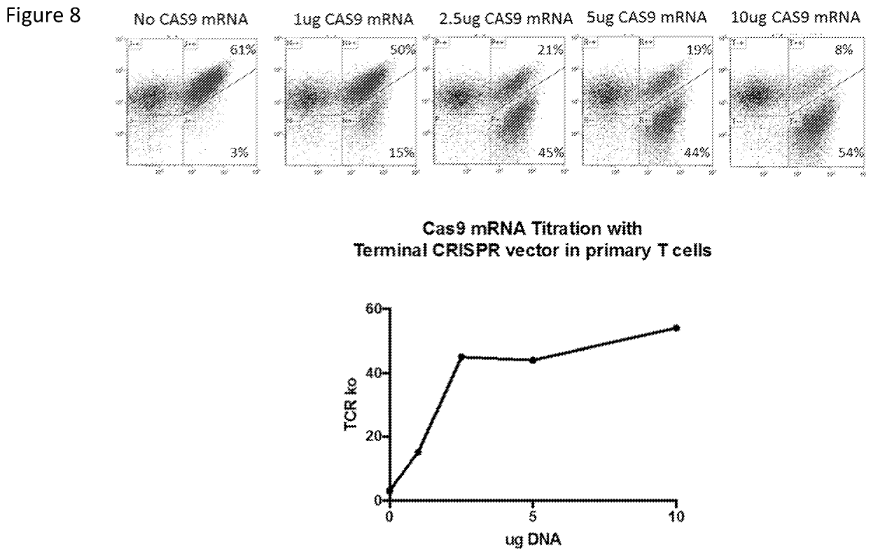

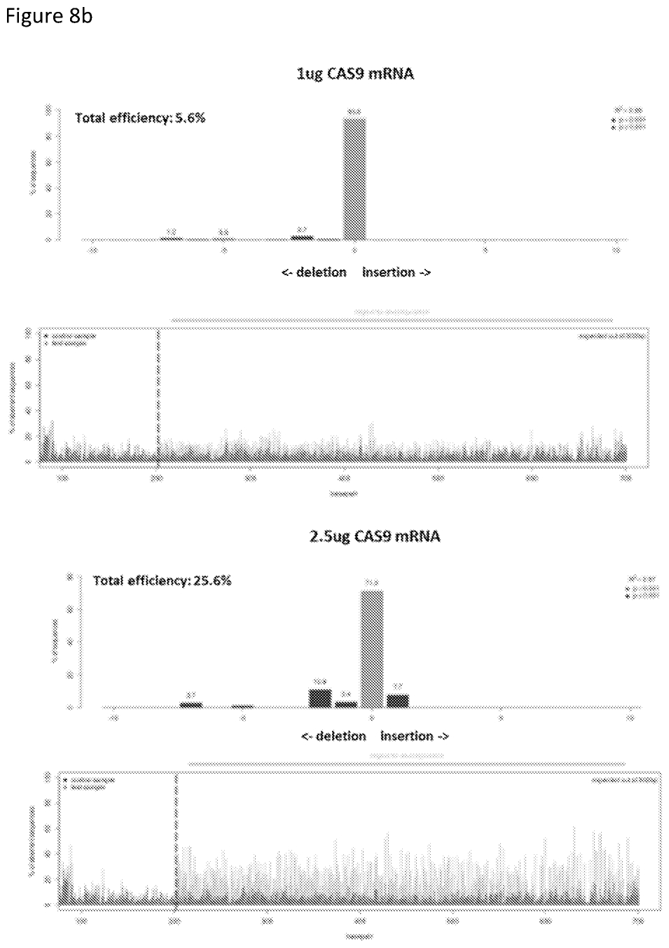

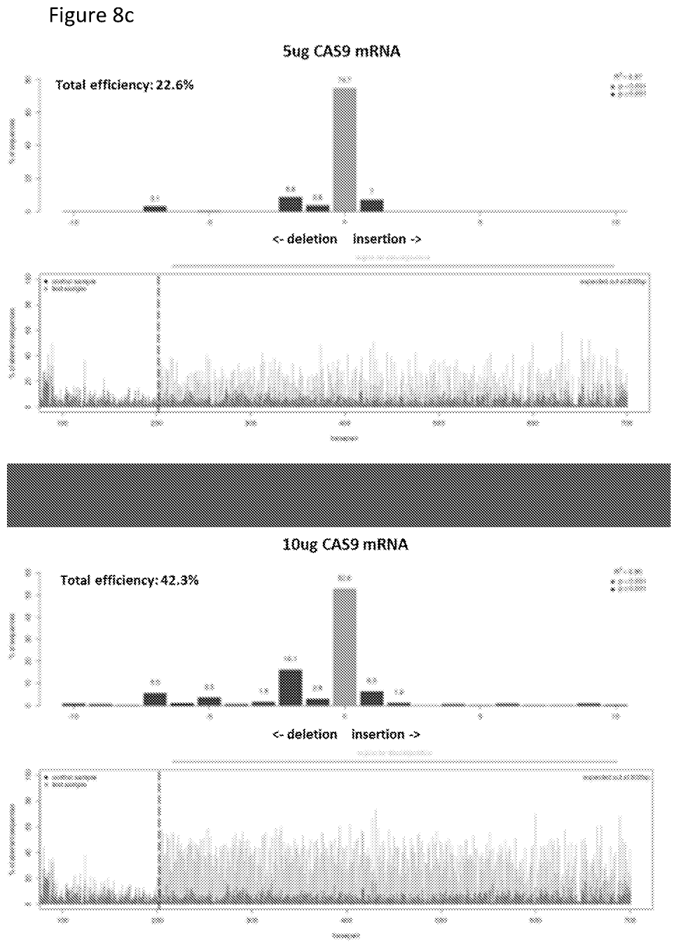

Cas mRNA was titrated in association with Terminal TRAC PGK CAR19 vectors (FIG. 8). Optimal Cas9 mRNA dosing range for effective TCR knockout was determined. Experiment undertaken using 10⁶ primary T cells from peripheral blood, transduced at MOIS after anti CD3/28 activation and then electroporated with Cas9 mRNA (Trilink, US) using a Neon electroporator. Flow cytometry for CAR expression (using anti-Fab) v TCR/CD3 expression is shown).

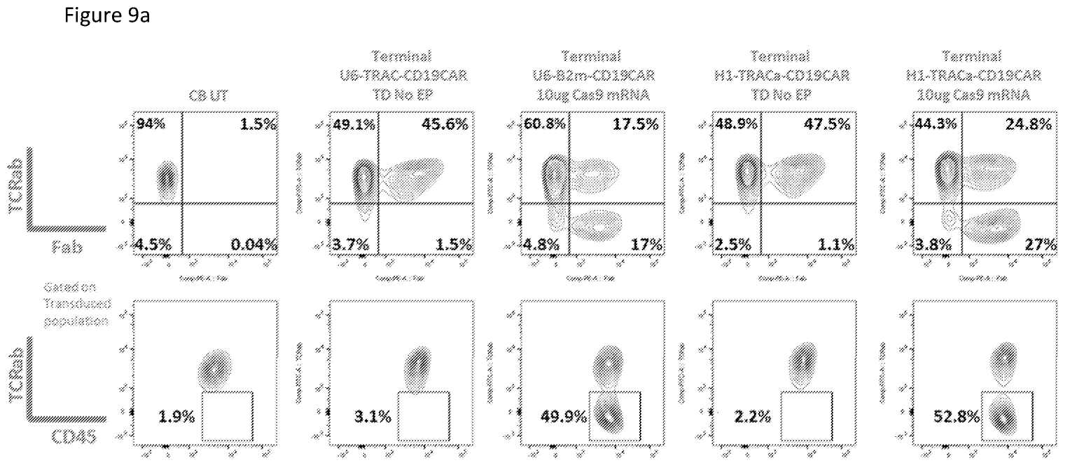

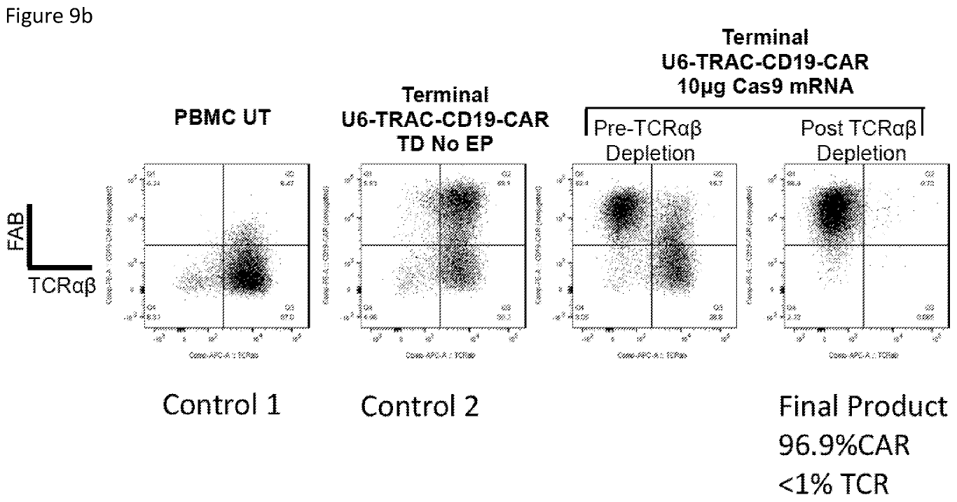

FIG. 9A relates to terminal TRAC CRISPR/PGK CAR19 in cord blood T cells. Cord cells were transduced after activation with anti-CD3/28. Upper panels show TCR expression and transduction (Fab stain for CAR) for both U6 and H1 configurations with and without electroporation of 10 ug of Cas9 mRNA.

FIG. 9B shows results from an experiment in which PBMC were thawed from a frozen leukapheresis from a healthy donor and activated for 24 hours with anti-CD3 and antiCD28 reagents as described in (Mock, Nickolay et al). Cells were then exposed in a clinical scale experiment to one round of transduction by Terminal U6-TRAC-CD19-CAR lentiviral vector before electroporation with Cas9 mRNA. The cells were then expanded for a further 7 days prior to TCRαβ depletion. C ells before and after TCR αβ depletion were assessed for expression of CAR by Fab staining and TCR αβ by flow cytometry alongside control untransduced (UT) PBMCs and additional control cells that had been transduced but not electroporated with Cas9 mRNA. The flow plots after processing by TCRαβ depletion reveal remarkable levels of 96.9% CAR transduced populations with <1% residual TCR expression. This was notably superior to previous manufacturing of similar universal T cell products using existing nuclease platforms, where CAR transduction is not linked to TCR knockout and Fab staining usually varies between 10-50%.

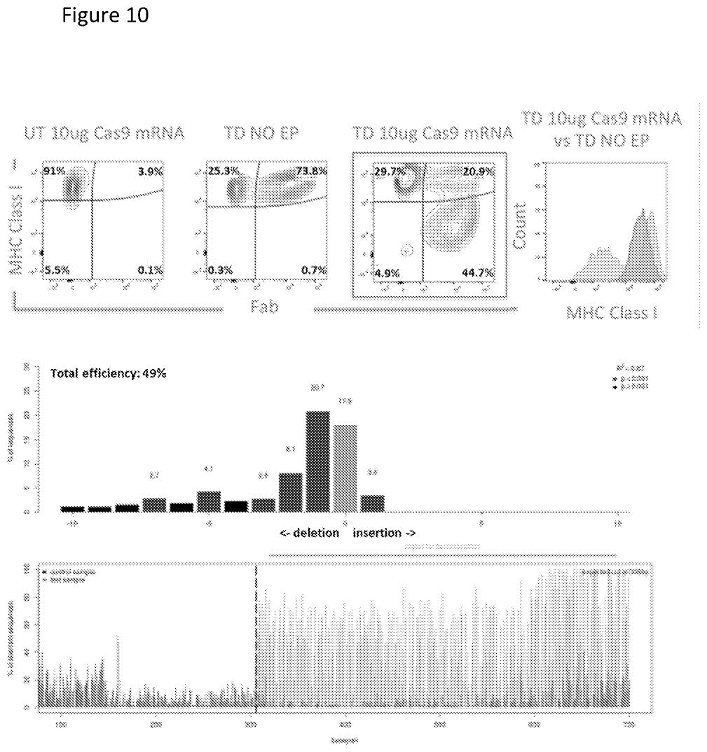

FIG. 10 relates to terminal β₂m CRISPR/PGK CAR19 in cord blood T cells. Cord cells were transduced after activation with anti-CD3/28, and then electroporated with 10 ug of Cas9 mRNA. Flow cytometry for HLA class I revealed efficient knockout which was restricted to the transduced populations. TIDE analysis confirmed disruption at the genomic level (49%), matching that observed by flow cytometry (45%).

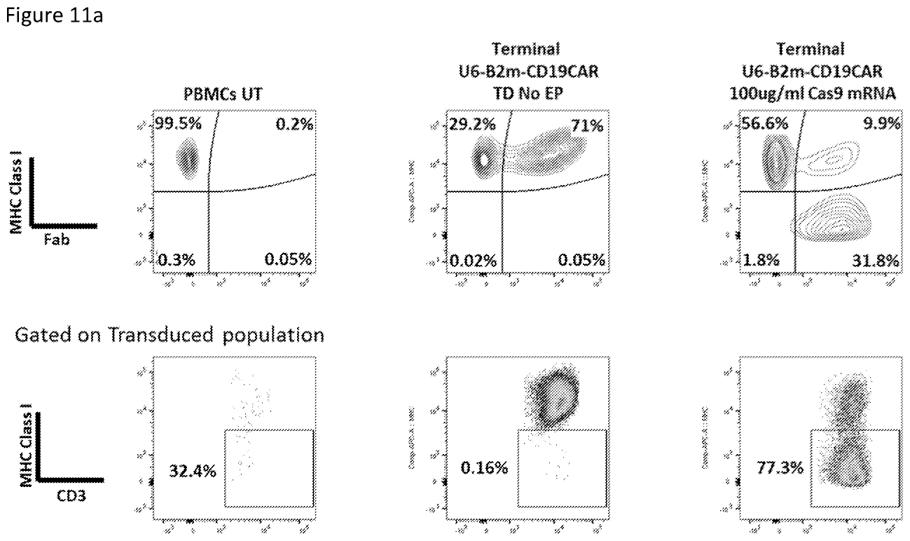

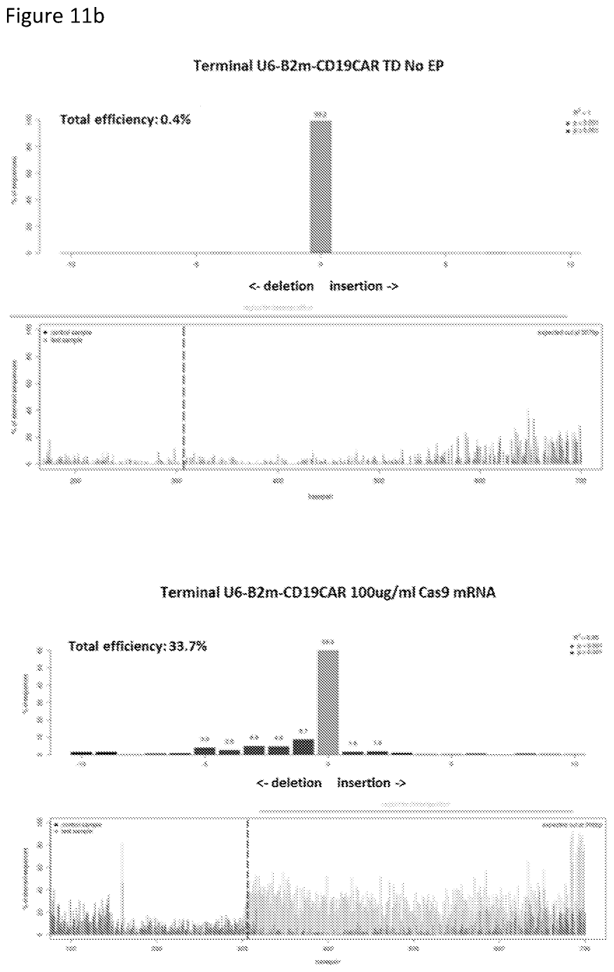

FIG. 11 relates to terminal β₂m CRISPR/PGK CAR19 in peripheral blood T cells. Peripheral blood monuclear cells (PBMCs) were thawed and rested overnight and then activated with antiCD2/CD28 before transduction with Terminal U6 β₂m CRISPR/PGK CAR19 on day 2 at MOI 5. 4×10⁶ cells were electroprated on day 5 in a BTX device with 20 ug Cas9 mRNA and assessed by flow on day 12. FIG. 11a showing disruption by flow cytometry in lymphocyte population with accompanying TIDE analysis (FIG. 11b). Middle panel showing disruption by flow cytometry restricted to the Terminal U6-β₂m CD19CAR transduced population.

### Example 2—Generation of T Cells Expressing Variant FcRIIIA

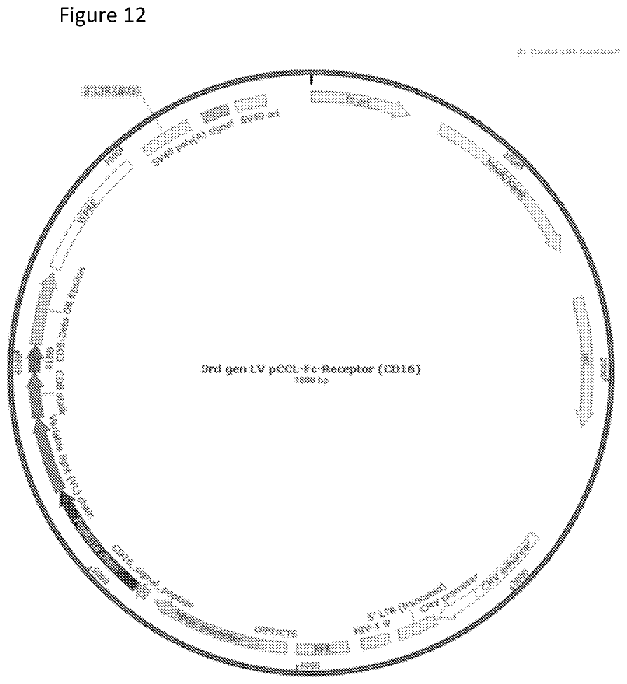

A schematic representation of a chimeric FcR (cFcR) vector plasmid is shown in FIG. 12. The vector is a third generation, integration competent but replication incompetent, self-inactivating lentivirus derived from HIV-1, with deleted U3 regions in the 3′LTR. Expression of a cFcR is driven by an internal promoter PGK. The vector incorporates a HIV central polypurine tract (CPPT) for nuclear entry and a mutated woodchuck postregulatory element (WPRE) for increased gene expression and titre. The cFcR includes a CD16 signalpeptide, human FcgRIIIa domain fused to an immunoglobulin light chain, CD8stalk and activation domains comprising 41BB and CD3ζ.

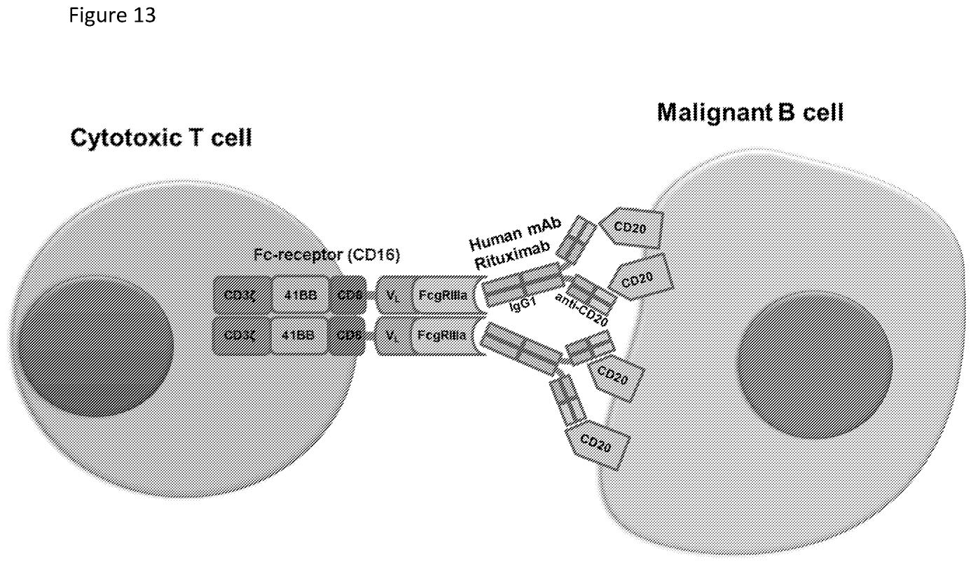

The function of T cells transduced to express cFcR is shown in FIG. 13. Transduced T cells engage the Fc domain of Rituximab, a widely used humanised monoclonal directed against the B cell antigen CD20, and mediated destruction of target cells. The incorporation of a light chain domain in the extracellular aspect of the receptor aims to foster dimerization and enhanced signalling potential. The configuration shown utilises a CD8 derived stalk and 41BB-CD3ζ activation domains.

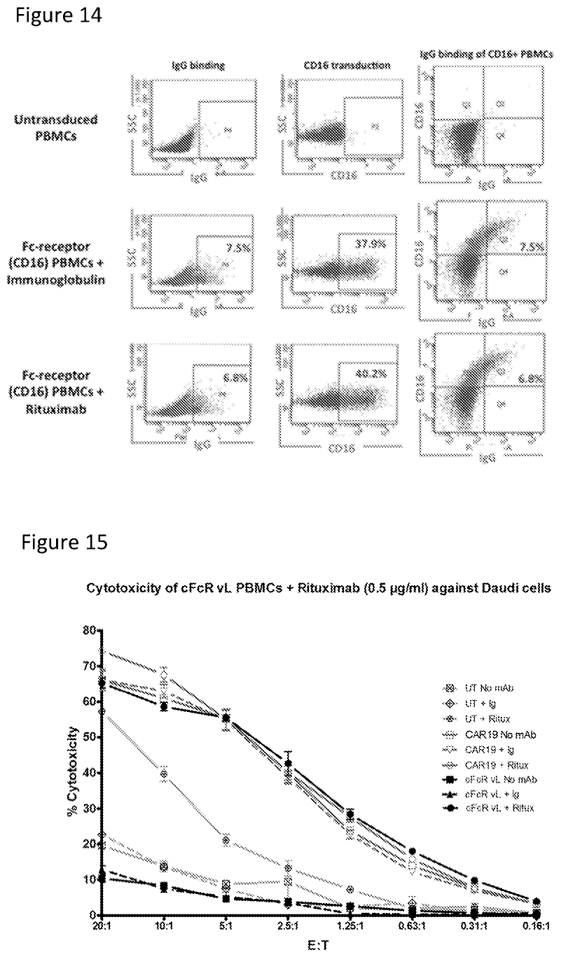

FIG. 14 demonstrates cFcR mediated binding of humanised IgG1 mAb. Human peripheral blood mononuclear cells (PBMC) were isolated by ficoll gradient centrifugation and activated with anti-CD3/CD28 stimulation. After a single round of lentiviral transduction, expression of FcR was confirmed by flow cytometry for CD16 (37.9% and 40.2%). Cells incubated with either human serum immunoglobulin (IgG) or anti-CD20 specific IgG (Rituximab) were examined by flow cytometry for CD16 and IgG co-staining (7.5% and 6.8% respectively)

FIG. 15 exemplifies cFcR mediated cytotoxicity of B cell tumour cell. In this experiment, CD19+20+ Daudi tumour cells were loaded with ⁵⁶Cr and exposed at various target:effector ratios to primary human T cells engineered to express cFcR or CAR19. Specific cytotoxicity was mediated by T cells expressing a CAR19 alone or in combination with IgG or Rituximab. In contrast cFcR T cells only mediated cytotoxicity in combination with Rituximab, and this was greater than cultures exposed to untransduced cells and Rituximab.

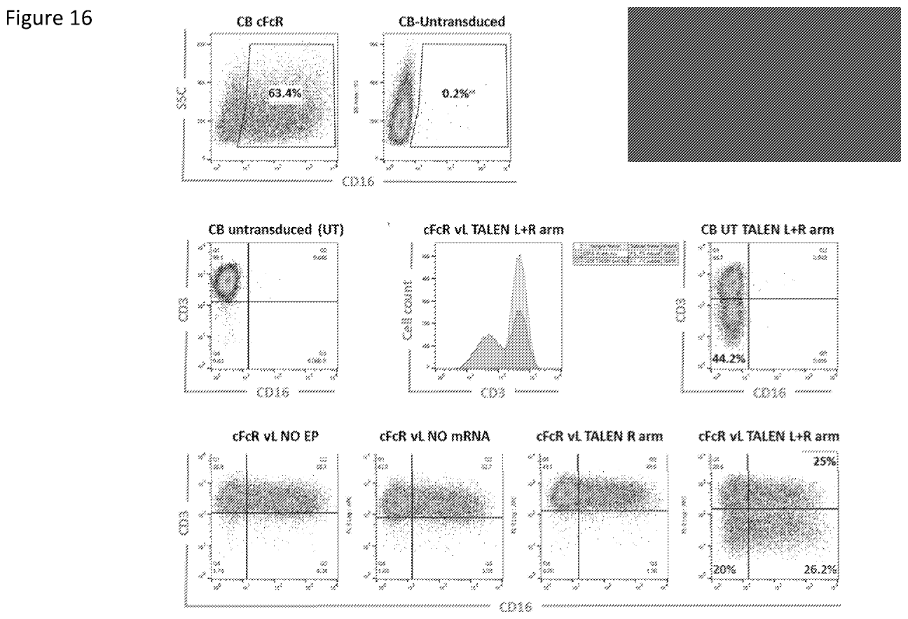

FIG. 16 shows that TCR may be depleted in cFcR T cells. Here, T cells were activated with anti-CD3/CD28 and exposed to a single round of lentiviral-cFcR transduction before electroporation with TRAC specific TALEN mRNA (left and right, alone or in combination). Transduction efficiency between 48-55% was achieved with over 45% TCR ko in samples treated with both TALEN arms.

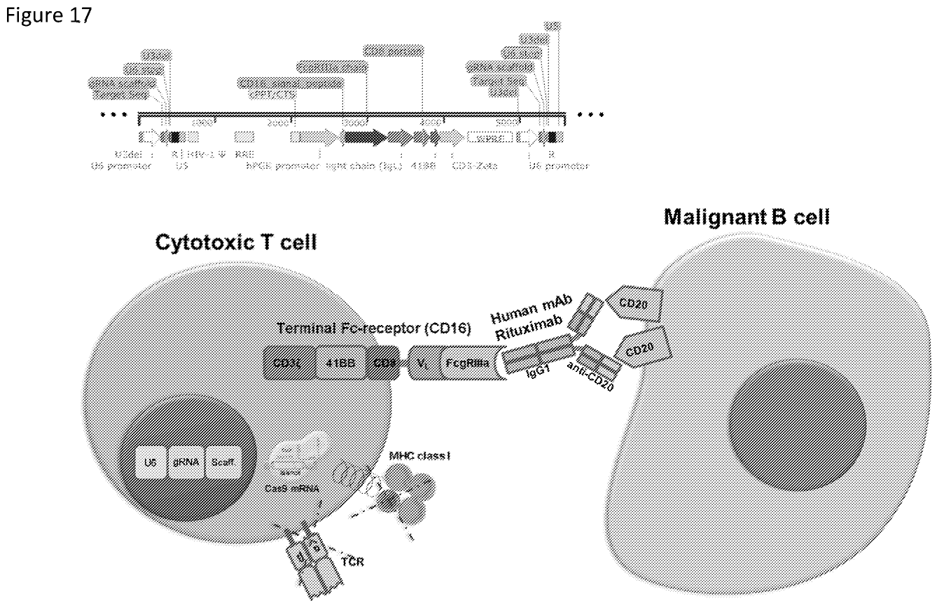

Terminal CRISPR vectors may be used for expression of cFcR and simultaneous CRISPR/Cas9 targeting of TRAC and β₂m (MHC class 1). A schematic is provided in FIG. 17.

### Background Methods for Examples 3 and 4

### Flow Cytometry

To phenotype cells, cells were stained with the following primary antibodies from Miltenyi Biotec unless otherwise stated, CD45 VioGreen, CD3-FITC, CD14-APC, CD20-APCVio770, CD56-PEVio770 and CD62L-APC. To assess the efficiency of CD19-CAR transduction, cells were stained using a Biotin SP (long spacer) AffiniPure F(ab) Fragment Goat Anti-Mouse immunoglobulin (Ig)G F(ab) Fragment specific antibody (Jackson Immunoresearch) followed by Streptavidin-APC (Biolegend). Cells were acquired on a 4-laser BD LSRII and flow cytometry analysis performed using FLowJo v10.

### Animated T Cell Processing (CliniMACS Prodigy)

Automated T cell Transduction was performed on the CliniMACS Prodigy using the TS520 tubing set and following the device and softwares instructions. Unless otherwise stated all materials and reagents were obtained from Miltenyi Biotec. For the T cell Transduction process including the CD62L pre-selection, fresh whole cord blood was sterile welded to the TS520 tubing set. CD62L microbeads were connected to the device and the CD62L selection process was initiated. The process incorporates a red blood cell depletion step followed by magnetic labelling and isolation of CD62L positive cells. The CD62L positive cells are automatically transferred to the re-application bag connected to the TS520 tubing set. Where cells had been processed using the Density Gradient Separation process the cells were collected in a transfer bag and sterile welded in the place of the re-application bag. All T cell Transduction process were initiated with at least 70×106 lymphocyte cells, based on a Sysmex count and were cultured in a total volume of 70 mls of TexMACS medium, 3% human serum (Sera Labs) and 20 ng/ml interleukin 2. Cells were activated using TransAct T cell reagent. The cells were transduced 24-48 hours post activation using a multiplicity of infection of 5 with a self-inactivating third generation lentiviral vector encoding a CAR specific for CD19, under the control of EF 1α internal promoter and including a mutated woodchuck post-regulatory element and human immunodeficiency virus central polypurine tract. The vector was pseudotyped with vesicular stomatitis virus. Sampling of the cells inside the culture chamber of the TS520 was performed daily and monitoring included sysmex based cell counting and flow cytometry. Cells were harvested from the machine after 8-9 days of cell expansion and were automatically formulated in CliniMACS PBS/EDTA buffer supplemented with 0.5% human serum albumin (Zenalb20; Bio Product Laboratory).

### Example 3—Processing of Cord Blood T Cells Using Density Gradient Separation

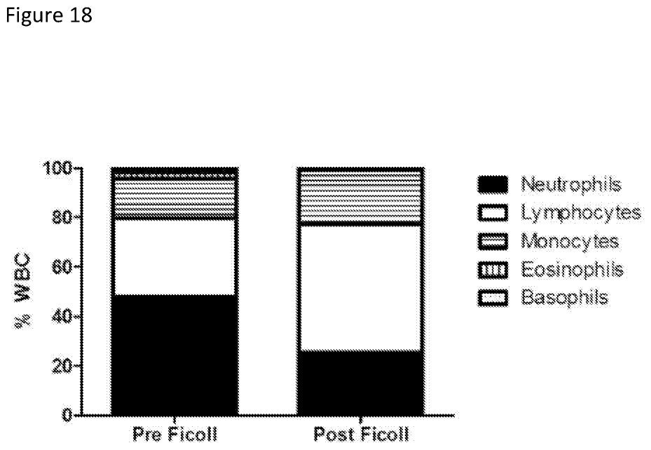

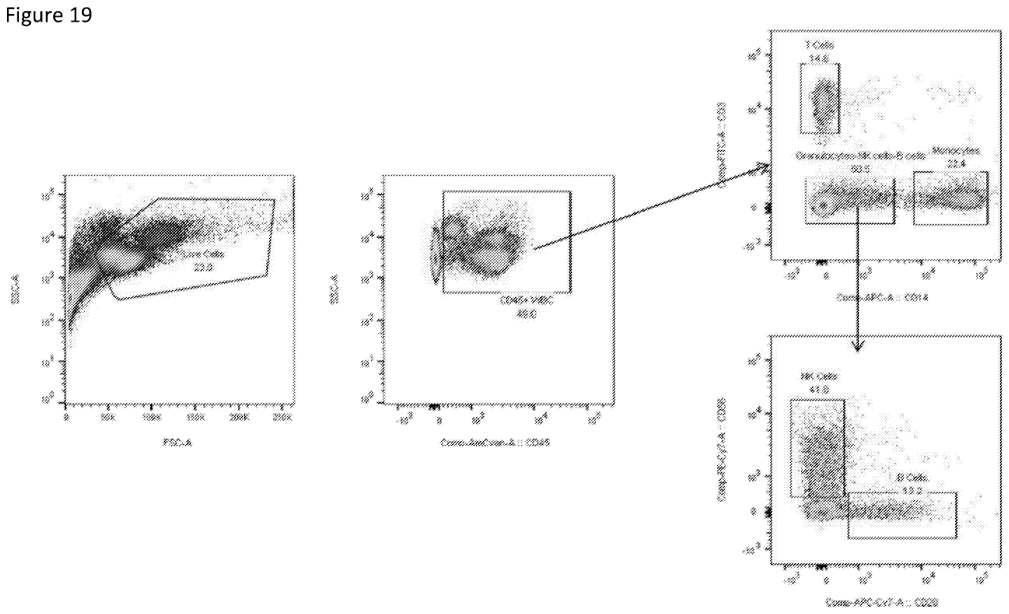

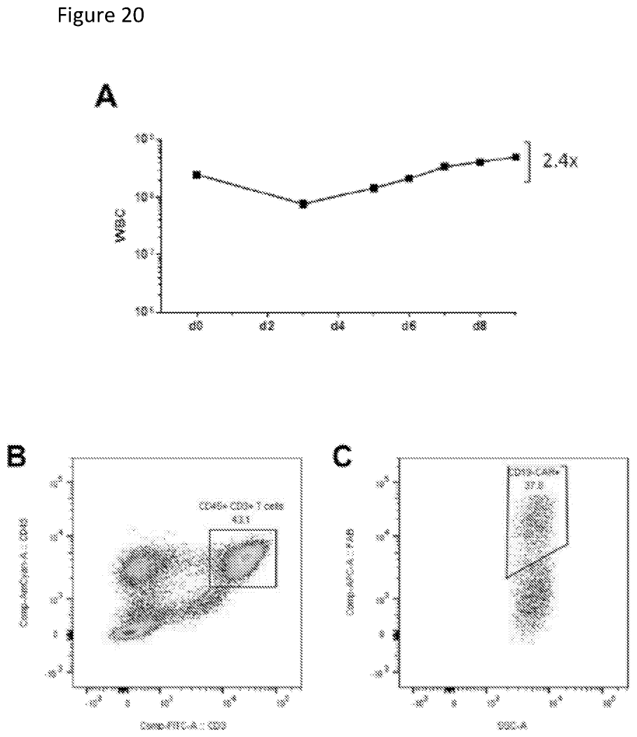

The T cell Transduction process on the CliniMACS Prodigy allows for the automated transduction and expansion of T cells. Our previous work has shown that this process can be used to generate a CD19-CAR T cell product from normal healthy peripheral blood leukapheresate (Mock, Nickolay et al). To investigate using the T cell Transduction process to engineer a T cell product from cord blood it was first critical to identify a means of isolating and enriched T cell population from whole cord blood. It is standard practice to enrich T cells from whole blood using density gradient separation (PMID 4179068). The Density Gradient Separation process on the CliniMACS Prodigy allows for the automated isolation of lymphocytes from whole blood (PMID 25647556). This process was performed using three cord blood donors to investigate if this process could be implemented to enrich T cells from whole cord blood. Samples of the cord blood were taken pre- and post-density gradient separation and a sysmex based method of cell counting, which can delineate white blood cell (WBC) populations based on the cell size and morphology, was used to analyse the cell population from cord blood. In our hands, although the lymphocyte subset was enriched from 32% of WBC to 51.6% of WBC using density gradient separation, a large proportion of granulocytes, namely neutrophils persisted (25.4% compared to 47.7% pre ficol) (FIG. 18). Post ficol WBC's were stained for antibodies against CD45, CD3, CD14, CD20 and CD56 to further assess the lymphoycyte subsets by flow cytometry (FIG. 19). Analysis of the CD45+ white blood cells showed a very low proportion of the target CD3+ T cell population of only 14.6%. This starting percentage of T cells from cord blood was much lower when compared to previous data using normal peripheral blood leukapheresate (Mock and Nickolay et al). Indeed, when cord blood cells processed using density gradient separation were expanded and transduced in the CliniMACS prodigy only a 2.4 fold expansion in WBC count was observed over a 9 day expansion period (FIG. 20A). Furthermore, the purity of CD45+CD3+ T cells in the expanded cord blood product was <50% and only a modest transduction with CD19-CAR LVV was observed (FIGS. 20B and C).

### Example 4—Processing of Cord Blood T Cells Using CD62L Selection

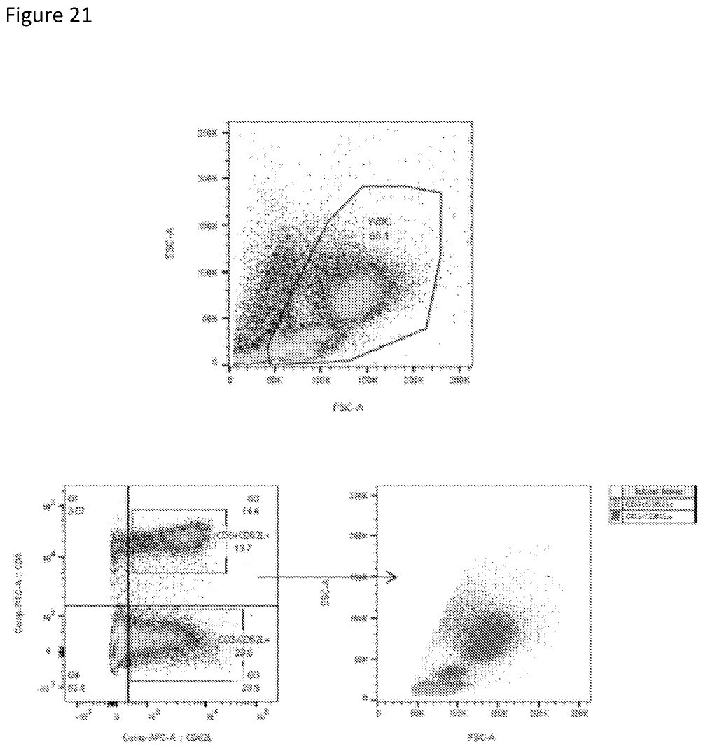

CD62L is a cell adhesion molecule which is expressed on naïve T cell to facilitate migration into secondary lymphoid tissues. In anticipation that cord blood T cells are almost all naïve, CD62L was identified a suitable cell surface molecule for isolating cord T cells and depleting non relevant populations that otherwise hamper cord processing (such as red cells, nucleated red cells, neutrophils, monocytes and other populations). Whole cord blood T cells were stained with antibodies against CD3 and CD62L to identify the populations of cells expressing CD62L (FIG. 21). Within the WBC population of cord blood cells, 17.47% were T cells, the majority of which expressed CD62L (82.4%) A proportion of CD3− cells also expressed CD62L and based on the FSC-A/SSC-A profile these cells are likely to be granulocytes.

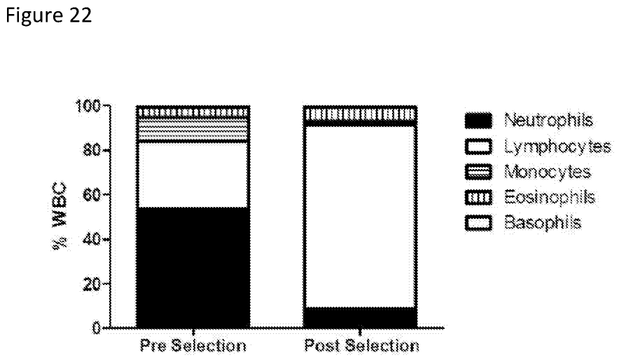

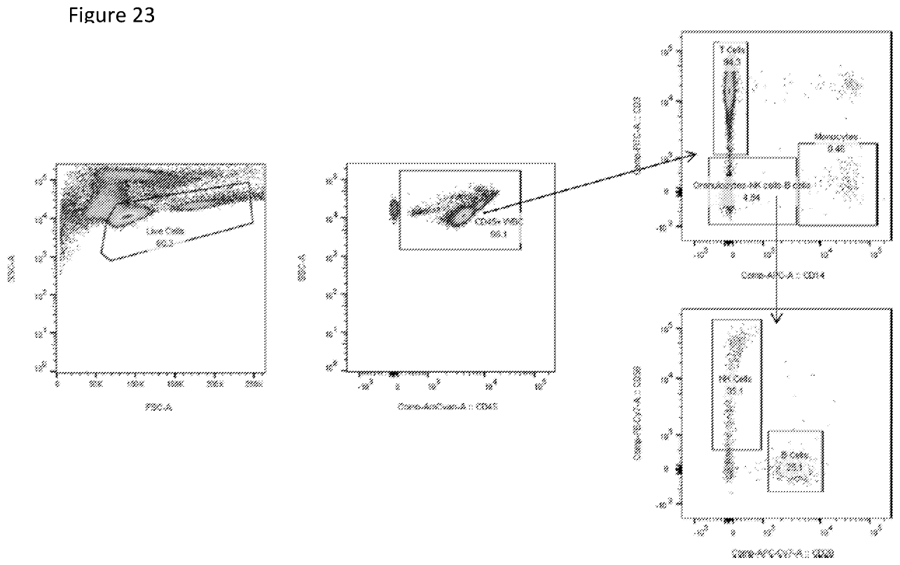

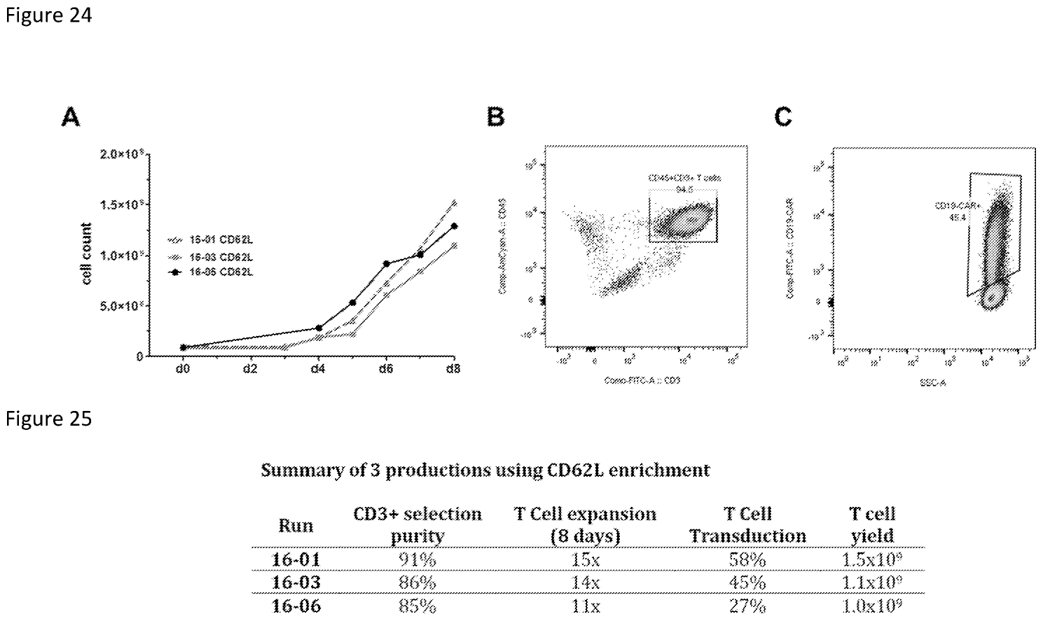

The T cell Transduction process on the CliniMACS Prodigy has an optional pre-selection step which was used to isolate CD62L positive cells. This pre-selection step was used to process three whole cord blood samples. In these three independent experiments we found that CD62L selection yielded a surprisingly enriched lymphocyte population from a mean of 30.2% to 82.3% based on sysmex method of cell counting (FIG. 22). Importantly other populations such as neutrophils were greatly reduced upon CD62L selection from a mean of 53.6% to 8.9%. Upon flow cytometric analysis of CD62L isolated cord blood cells we identified that CD45+ WBC were greatly enriched, 96.1% compared to cord blood cells that were processed using density gradient separation (49.1%) (FIGS. 19 and 23). Importantly, the majority of the CD45+ cells were CD3+ T cells, with very few residual monocytes, neutrophils, B cell and NK cells. The CD62L selected cord blood cells were used as the starting cell population for the T cell Transduction process on the CliniMACS Prodigy. Over a period of 8 days the mean fold expansion of cord blood cells was 13.4× (FIG. 24A). The expanded cells were of a high CD45+CD3+ purity and were efficiently transduced with a LLV encoding CD19-CAR (FIGS. 24B & C). A summary of the three T cell Transduction processes performed using CD62L selected cord blood cells is shown in FIG. 25. Critically, in each of the three runs target yields of >1×10⁹ total cells and transduction efficiency of >10% was achieved, which are typical specifications required for final level dosing for clinical application.

### Example 5—CRISPR-CAR Coupled Universal T Cells Mediate Potent Anti-Leukaemic Effects

### Summary

Gene editing offers the possibility of generating T cells that can overcome HLA barriers to mediate invigorated immune effects. Initial therapeutic applications have included the production of universal T cells expressing chimeric antigen receptors against leukaemia antigens such as CD19. Current approaches rely on stable vector mediated transfer of a CAR expression cassette and transient nuclease mediated DNA scission at targeted loci such as the T cell receptor alpha constant chain (TRAC). In the absence of coupling, transgene expression and editing effects are unlinked, yields are variable and the resulting T cells populations are heterogenous. In this Example, we report a self-inactivating lentiviral vector platform that couples CAR expression with CRISPR/Cas9 effects through a hybrid terminal-TRAC guide element incorporated into the U3 3′long terminal repeat. Duplication of pol III-sgRNA following reverse transcription magnifies expression, and subsequent electroporation mediated delivery of Cas9 mRNA mediates transient DNA cleavage. The ‘terminal’ CRISPR configuration, used in combination with downstream depletion of residual TCR expressing cells, yielded highly homogenous populations where terminal-TRAC CAR cells were >96% CAR+ and >99% TCR−. In vitro cytotoxicity studies confirmed functional integrity of the cells and in vivo anti-leukaemic effects in humanised immunodeficient mice were superior to conventional lentiviral-CAR transduced cells, with less exhaustion and reduced tumour burden.

### Introduction

T cells engineered to express recombinant antigen specific receptors or chimeric antigen receptors in early phase trials, with some approaches yielding compelling remission effects against refractory leukaemia. The majority of subjects treated to date have provided and received autologous T cells, but this approach may not be best suited for widespread cost-effective delivery of cellular therapy. Gene editing offers the prospect of addressing HLA-barriers and the development of universal T cell therapies. Recently, T cells modified using transcription activator-like effector nucleases (TALENs) and expressing chimeric antigen receptor (CAR) against CD19 have been used to treat refractory relapsed B cell acute lymphoblastic leukemia (B-ALL) in infants. These ‘off-the-shelf’ cells were derived from a non-HLA matched donor and were disrupted for CD52 expression to evade the depletion effects of Alemtuzumab, and were simultaneously modified at the T cell receptor (TCR) alpha chain constant (TRAC) region chain and depleted of T cells expressing TCRab to reduce the risk of graft versus host disease (GVHD). Clinical trials are underway to assess the strategy further in children and adults, and key aspects determining dosing schedules relate to carriage of residual TCRαβ T cells and proportion of cells expressing CAR19. The former comprise <1% of the total cell inoculum after TCRαβ magnetic bead depletion, but constitute a risk for GVHD and are strictly capped to below 5×10⁴ T cells/kg. This in turn limits the total cell dose, and because only a proportion of cells express CAR19 as a result of batch-to-batch variation in lentiviral transduction efficiency, the total cell dosing regimen differs between batches.

This sin-lentiviral platform couples transgene expression with CRISPR editing effects for efficient and homogenous T cell modification. CRISPR mediated effects in CAR19 modified T cells have been reported previously. For example, Ren et al. used CRISPR RNA electroporation to disrupt endogenous TCR and B2M genes for disruption of MHC class I in T cells transduced with a lentiviral CAR vector, but editing and transgene effects remain unlinked. Certain lentiviral configurations have incorporated both CRISPR guide sequences and Cas9 expression cassettes which become integrated into the target cell genome as a constituent of proviral vector DNA. While suitable for pre-clinical studies, constitutive expression of Cas9 may be problematic in human trials, not least because of its bacterial origin and possible immunogenicity. In order to overcome this issue, we delivered Cas9 mRNA by electroporation to T cells that had been transduced with a ‘terminal-CRISPR’ sin-lentiviral configuration. Here, CAR19 was expressed under the control of an internal human promoter and CRISPR guide sequences and associated H1/U6 promoters were incorporated into the U3 region of 3′ long terminal repeat (LTR) sequence of the vector. This configuration had a number of advantages, including avoiding interference effects that may effect vector genome expression during vector production or transgene expression levels following transduction. In addition, guide duplication and incorporation into the 5′LTR during reverse transcription and linkage of guide effects to cells encoding vector and expressing transgene was anticipated. Here, we demonstrate the potential of the platform for the generation of universal human T cells, including compliant scalability and demonstrate the advantages of TCR depleted CAR19 T cells (TCR− CAR19+ T cells) in a human:murine chimeric tumour model.

### Material and Methods

### CRISPR-Cas Vector System

A pCCL derived third generation SIN lentiviral vector incorporating a HIV-a cPPT elements and mutated WPRE for the expression of a CAR19 transgene under the control of a human PGK promoter was subjected to site direction mutagenesis to remove BbsI, BsmbI and SapI restriction sites using a QuickChange Lightning Kit (Agilent Technologies). U6 and H1 CRISPR guide cassettes were then cloned into the ΔU3 region of the 3′LTR using In-Fusion PCR Cloning Plus (Clontech). Single guide RNA (sgRNA) for TRAC (TCTCTCAGCTGGTACACGGC; SEQ ID NO: 1) cloned into the terminal vector was designed against reference sequences data (http://www.ensembl.org) using the Massachusetts Institute of Technology (MIT) CRISPR Design tool (http://crispr.mit.edu). CRISPR cassettes were designed with flanking Xba1 restriction sites to accommodate easy switching. Additional BbsI restriction sites were then incorporated between Pol III promoter and scaffold sequences to allow for efficient guide sequence substitution. CRISPR cassettes were synthesized by GeneART (ThermoFisher Scientific) based on the Zhang group Streptococcus pyogenes Cas9 scaffold sequence. Vector stocks were produced by transient transfection of 293T cells using a four plasmid system and concentrated by ultracentrifugation.

mRNA Cas9

CleanCap Cas9 mRNA (SF370, TriLink biotechnologies, US) expressed Streptococcus pyogenes Cas9 and incorporated nuclear localisation signals at both N and C terminus and co-transcriptional capping supported a naturally occurring Cap 1 structure which in conjunction with polyadenylation and modified uridine optimised mRNA Cas9 expression and stability. mRNA was delivered by electroporation by the Neon transfection system (ThermoFisher), Lonza 4D or BTX device in accordance with manufacturer's instructions. Cells were incubated at 30° C. overnight after electroporation before restoration to 37° C.

### Primary Human Lymphocyte Culture and Modification

Peripheral blood mononuclear cells (PBMCs) were isolated by ficoll density gradient and subsequently activated with TransACT reagent (Miltenyi Biotec). Lymphocytes were cultured in TexMACS medium (Miltenyi Biotech) with 3% human AB serum (Seralabs) and 100 U/ml IL-2 (Miltenyi Biotec). Transduction with lentiviral vector was performed day 1 after activation at a multiplicity of infection (MOI) of 5 and Cas9 mRNA electroporation performed 3 days later. Lymphocytes were cultured until day 11 post activation, by which time they were cryopreserved in 90% FCS and 10% dimethylsufoxide (DMSO). Scaled experiments ultilised a part-automated. Briefly, a T-cell transduction (TCT) program was adapted on the CliniMACS Prodigy using the Tubing Set TS520 and used cryopreserved leukapharesis harvest (Allcells, US) cultured in TexMACS GMP Medium supplemented 3% HS+20 ng/ml MACS GMP Human Recombinant IL-2. Cells were activated with MACS GMP TransAct CD3/CD28 Kit at a final dilution of 1:200 (CD3 Reagent) and 1:400 (CD28 Reagent. Cells were transduced after 24 hours.

### Flow Cytometry

Cells acquired on a 4-laser BD LSRII and FACS analysis performed using FlowJo v10 after being stained with the following primary antibodies against CD3, CD4, CD8, CD45, CD20, CD56, PD1. To assess the efficiency of CD19-CAR transduction, cells were stained using a Biotin-SP (long spacer) AffiniPure F(ab′) Fragment Goat Anti-Mouse IgG, F(ab′) Fragment Specific antibody (Jackson Immunoresearch) followed by Streptavidin-APC or Streptavidin-FITC (Biolegend).

### Detection of Non-Homologous End Joining Events

Genomic DNA extraction was performed using DNeasy Blood and Tissue Kit (QIAGEN) and a PCR reaction designed to amplify 700-800 bp around sites of predicted Cas9 scission. Primers were TRAC forward: TTGATAGCTTGTGCCTGTCCC (SEQ ID NO:2), TRAC reverse: GGCAAACAGTCTGAGCAAAGG (SEQ ID NO: 3) and reactions used Q5 High-Fidelity DNA Polymerase (New England BioLabs) on an Alpha Cycler 4 (PCRmax). PCR products were discriminated by 1% agarose gel electrophoresis, sequenced and analysed using Tide protocols (https://tide.nki.nl/). In addition, 200 ng of PCR product were heated to 95° C. before cooling, digestion with T7 Endonuclease I (New England BioLabs) and gel electrophoresis.

### Cytokine Secretion

Effector cells (CD19CAR+) were thawed in Roswell Park Memorial Institute medium (RPMI) with 10% FCS and re-suspended in a 1:1 ration with CD19 target cells (CD19+ SupT1 cells) and controls (CD19− SupT1 cells) in a 24 well format. After incubation at 37° C. for 24 hours, supernatant was filtered and cryopreserved for subsequent T_{H}1/T_{H}2/T_{H}17 cytometric bead array kit (CBA; BD Biosciences).

### In Vitro Cytotoxicity

Cytotoxic function of both TCR+ CAR19+ T cells and TCR− CAR19+ T cells was assessed by incubating CD19+ SupT1 target cells labelled with ⁵¹Cr, with effector cells (CD19CAR+) at increasing effector to target ratios (E:T in a 96 well format for 4 hours at 37° C. Release of ⁵¹Cr was quantified using a microplate scintillation counter and specific cytotoxicity calculated as previously described.

### In Vivo Anti-Tumour Activity

NOD/SCID/γc^{-/-} (NSG) mice, were inoculated with 5×10⁵ CD19+ Daudi tumour cells by tail vein injection on day 0. The tumour cells had been stably transduced to express GFP/Luciferase. Tumour engraftment was confirmed by in vivo imaging of bioluminescence using an IVIS Lumina III In Vivo Imaging System (PerkinElmer, live image version 4.5.18147) on day 3. On day 4 animals were injected with either PBS (n=3), 5×10⁶ untransduced T cells (n=8), 5×10⁶ TCR+ CAR19+ T cells (n=8) or 5×10⁶ TCR− CAR19+ T cells (n=8). Follow up in vivo imaging of bioluminescence was carried out on days 7, 10, 14, 18, 21, 28, and 35. Bone marrow and spleen samples were processed by a red blood cell lysis followed by staining for flow cytometry.

### Results

### Lentiviral Terminal-TRAC (TT) Guide RNA Vectors

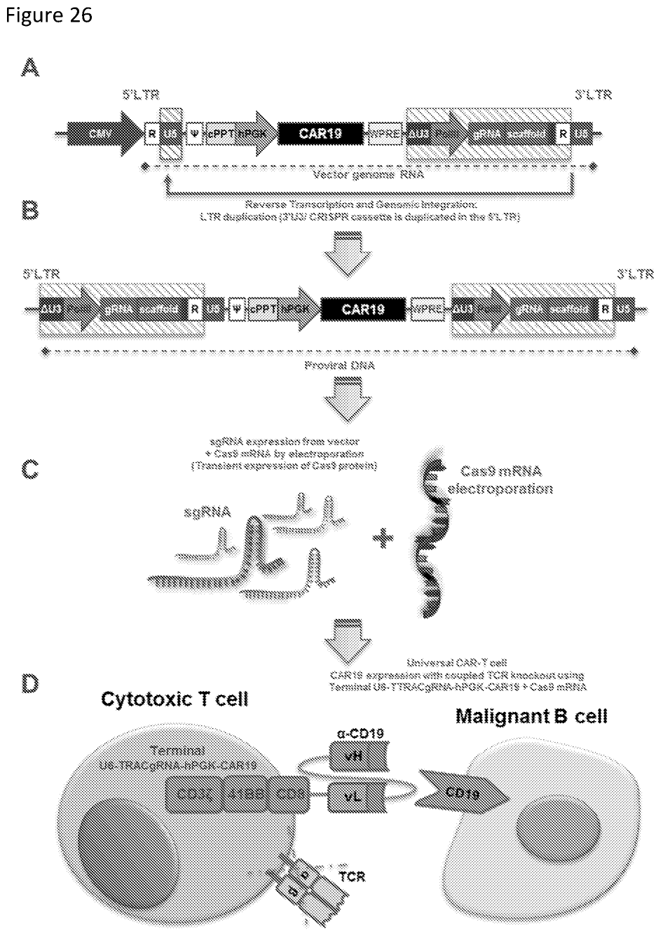

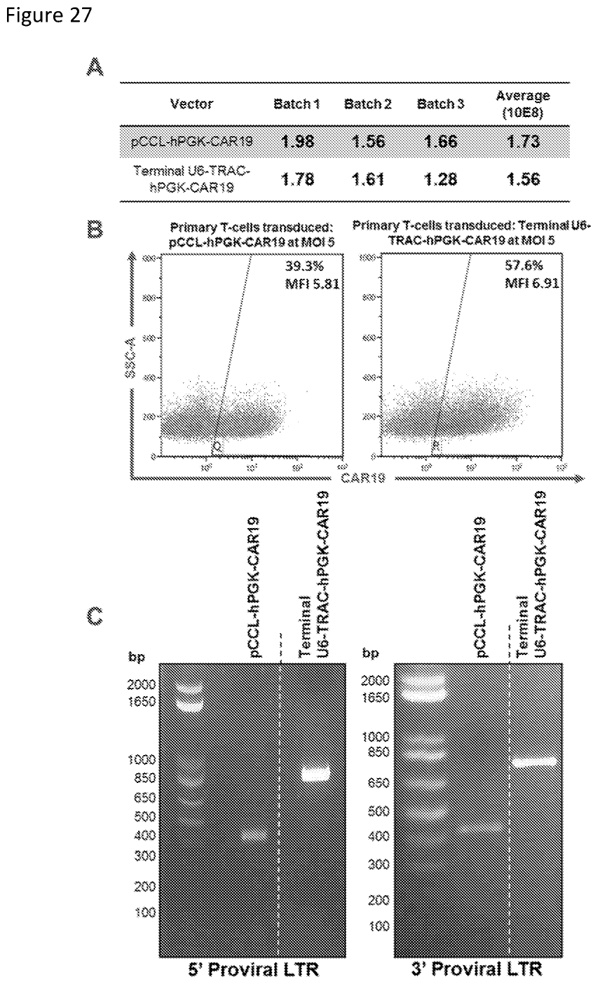

Incorporation of a pol III promotor and sgRNA sequence into the 3′ LTR of a U3 deleted third generation lentiviral vector, generated a self-duplicating CRISPR expression cassette (FIG. 26). A region immediately proximal to 3′ repeat (R) regions was selected to preserve reverse transcription mediated duplication effects to the 5′ LTR, resulting in a proviral form with both 5′ and 3′ flanking terminal CRISPR elements. This increased expression levels of sgRNA (confirmed by RT-qPCR) without risk of interference between internally sited RNA PolIII promoters and transcriptional activity of any transgene promoter. For targeting of endogenous TCR expression, a sgRNA sequence targeting the TRAC locus was placed under the control of the human PolIII promoter, U6 followed by a scaffold (tracrRNA) sequence specific for S.pyogenes Cas9 in a lentiviral construct encoding a CD3z-41BB-CD8-CAR19scFv (4G7) chimeric antigen receptor (CAR19) under the control of an internal human phosphoglycerate kinase (PGK) promoter. Expression of the CAR19 transgene in primary T cells (MOI 5) was 57% for TT-TRACa-hPGK-CAR19 vector (MFI 6.91) compared to 39% (MFI 5.81) for the conventional pCCL-hPGK-CAR19 (FIG. 27A). Unique primer pairs were used to amplify both 3′ and 5′ LTR regions in these cells (FIG. 27B). The 3′ U5 reaction amplified the expected 392 bp product from the pCCL-hPGK-CAR19 transduced cells compared to a larger 755 bp product from the TT-hPGK-CAR19 transduced cells, and the 5′PCRs confirmed a larger 742 bp PCR product indicating duplication of the U6 promoter-sgRNA-scaffold sequences compared to the smaller 379 bp conventional duplication and these results were verified by Sanger sequencing.

Transient Cas9 mRNA Delivery by Electroporation to Terminal-TRAC T Cells

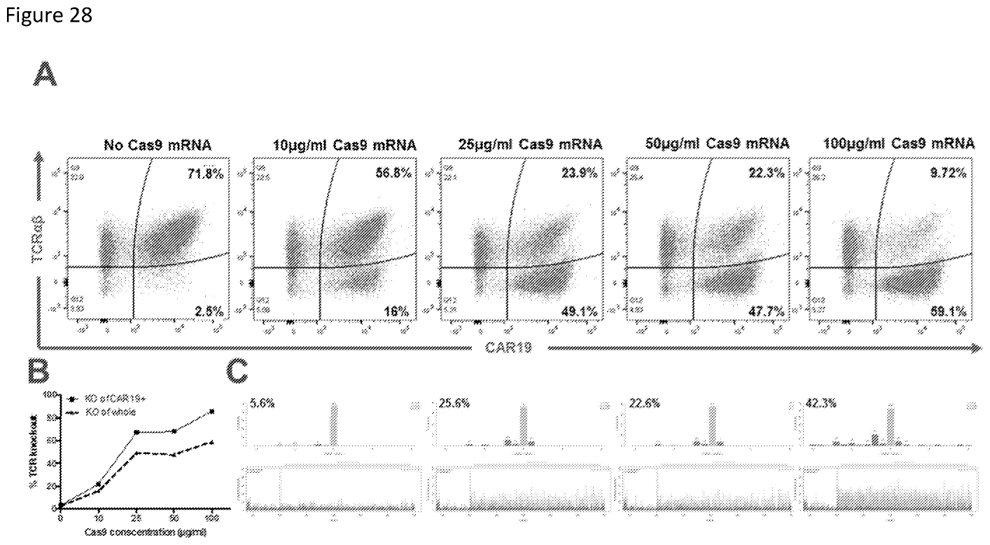

Incorporation of CRISPR sgRNA into the terminal regions of a CAR encoding lentiviral vector coupled expression of these elements, but DNA scission still required Cas9. Previous lentiviral configurations have incorporated Cas9 expression cassettes, but this could result in ongoing scission effects, increase the risk of off-target toxicity and confer immunogenicity. We delivered Cas9 mRNA by electroporation for transient effects in dividing T cells 1, 2, 3, 4 or 7 days after exposure to a single round of transduction with TT-hPGK-CAR19 vector and found 3 days to be optimal for TRAC disruption. Dilution and elimination of Cas9 protein was confirmed by serial Western blot analysis. When Cas9 mRNA was titrated (0 μg/ml-100 μg/ml) and disruption of TCR was quantified 10 days later by flow cytometry (FIG. 28A), a plateau effect above 25 ug/ml was revealed (FIG. 28B). Molecular signatures of NHEJ at the TRAC locus were confirmed by TIDE analysis of a 772 bp TRAC amplicon (FIG. 28C), and were consistent with flow cytometry data given that TRAC locus allelic exclusion is in operation in the majority of T cells. Crucially, only cells expressing CAR19 were found to be TCR negative, confirming CRISPR effects were coupled to transgene expression.

### Scalability of Terminal-TRAC T Cell Production

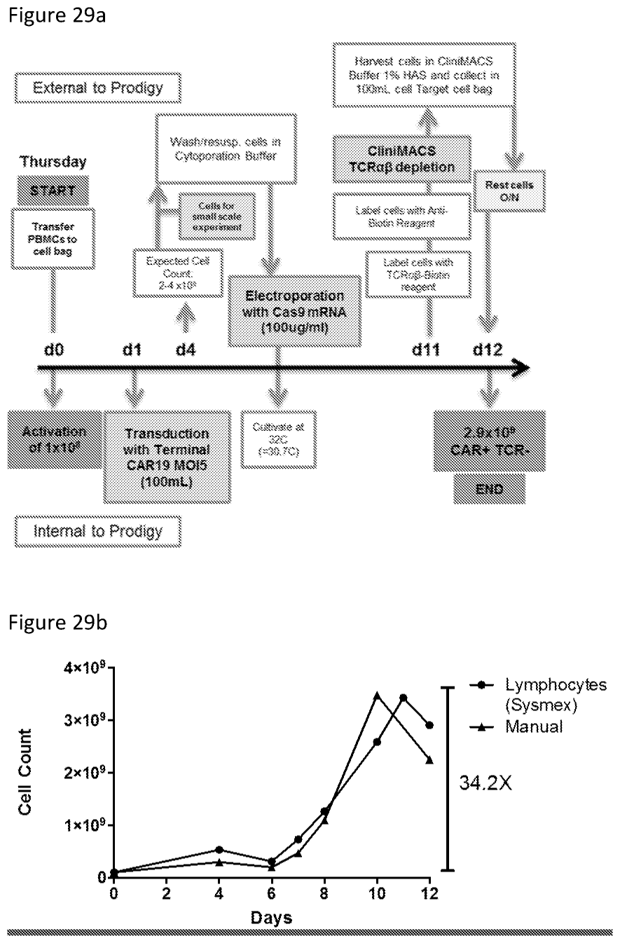

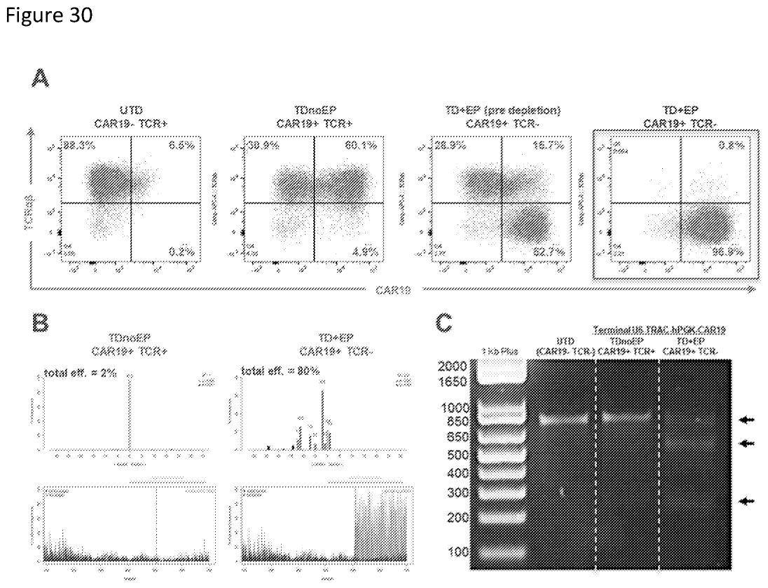

A critical hurdle for CRISPR/Cas9 gene editing is scalability and compliance for therapeutic manufacturing. We adapted theautomated T cell lentiviral transduction procedure using the CliniMacs Prodigy system alongside conventional cultures in GRex flasks, and activated 100×10⁶ thawed PBMCs with soluble CD3/CD28 Transact reagent ahead of lentiviral transduction with TT-hPGK-CAR19 vector at M015 in the closed system tubing cell (FIG. 29A). After a further 72 hr, cells were removed from the device and Cas9 mRNA was electroporated, and the cells cultured overnight at 30° C. before returning to the Prodigy. After 11 days, CAR19 expression was 62% in CD45+CD2+ cells and TCR knockout in CD45+CD2+CAR19+ cells was 77%. Further processing by magnetic bead mediated depletion of residual TCRab cells yielded a highly purified population of TCR depleted cells (>99%), almost all (96.9%) of which were CAR19+ T cells (FIG. 30A). Cells were characterised in detail by flow cytometry. Disruption of the TRAC locus at the genomic level was verified by a T7 nuclease (FIG. 30C) assay and by TIDE PCR (FIG. 30B) which revealed NHEJ events of around 80%. Overall, a final cell yield of 2.9×10⁹ Terminal TRAC TCR− CAR19+ T cells were produced (FIG. 29B), almost 30× the starting number, and sufficient to create therapeutic doses for over 20 average adult subjects.

### Terminal TRAC TCR− CAR19+ T Cells Efficiently Target CD19+ Cells In Vitro

As set out above, if terminal CRISPR is used to disrupt TRAC expression in the formation of TCR− CAR19+ T cells, the resultant TCR− CAR19+ T cells may be referred to as terminal TRAC TCR− CAR19+ T cells (TT TCR− CAR19+ T cells).

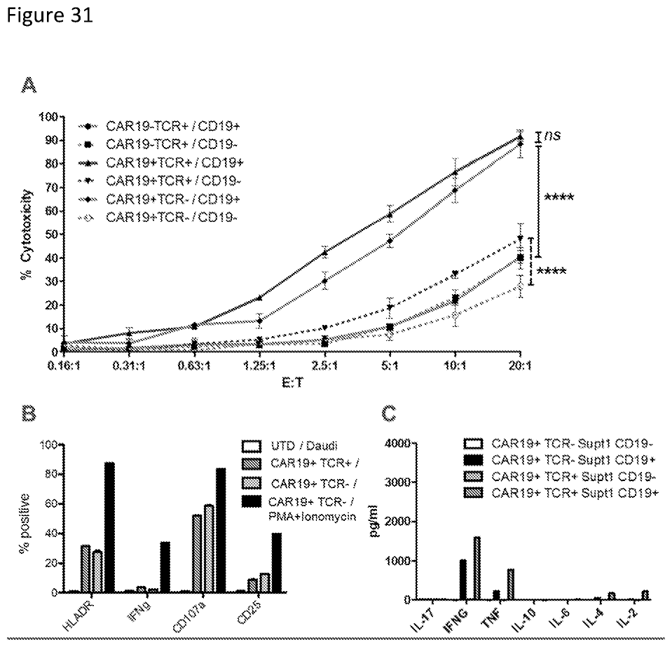

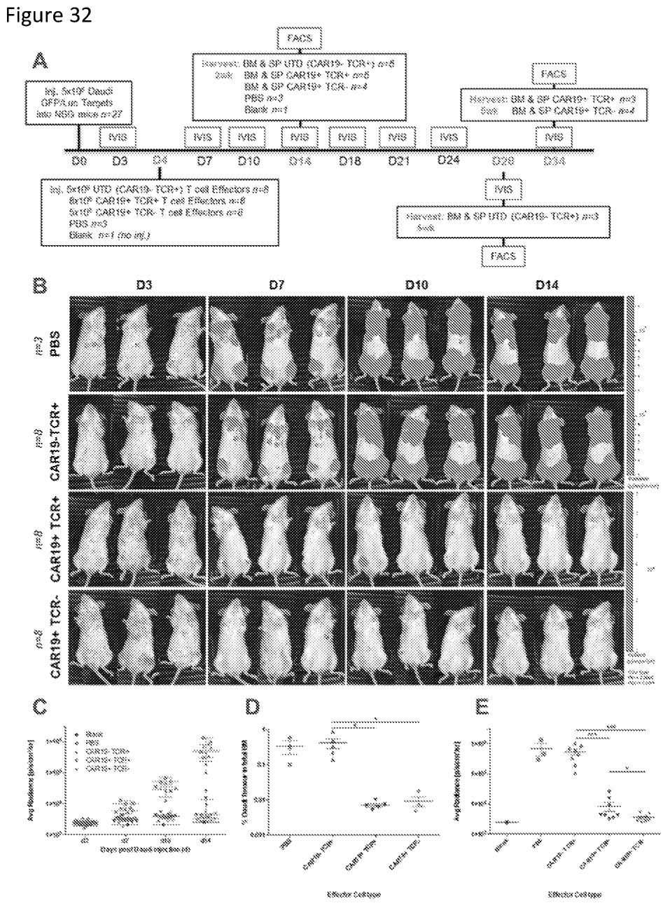

The cytolytic potential of TT TCR− CAR19+ T cells was assessed in an in vitro cytotoxicity assay against ⁵¹Cr loaded CD19+ or CD19− SupT1 target cells. Both TT TCR− CAR19+ T cells and TT TCR+ CAR19+ T cells exhibited rapid and efficient specific lysis of CD19+ targets after 4 hr of co-culture, in contrast to non-transduced CAR19−TCR+ effectors (P<0.0001) (FIG. 31A). We noted that TCR− CAR19+ T cells exhibited low level cytotoxicity irrespective of target CD19 suggestive of background TCR mediated allo-recognition. Lysis of another CD19+ tumour line, Daudi, was also documented for CAR19+TCR+ and TT TT TCR− CAR19+ T cells effectors. A degranulation assay carried out on the CD19+ Daudi target cell line further corroborated the cytotoxicity data with 57% of TCR− CAR19+ T cells and 53% of TCR+ CAR19+ T cells upregulating CD107a expression after a 4 hr incubation of effectors with target FIG. 31B). Finally, a cytometric bead array assay recorded increases cytokine production of IFNg, TNF, IL-4 and IL-2 after 12 hr from CD19+Supt1 co-cultures but not from CD19− Supt1 targets further confirming specificity (FIG. 31C).

### Terminal TRAC Anti-Leukemic Activity In Vivo

A humanised murine model of leukaemic clearance was used to assess in vivo function of engineered CAR19 T cells. NSG mice inoculated intravenously with 5×10⁵ CD19+EGFP+Luciferase+ Daudi cells were imaged after 3 days and then in groups of 8 animals, injected with effector cells comprising TT TCR− CAR19+ T cells, TCR+ CAR19+ T cells or TCR− CAR19+ T cells +. Serial luminescent imaging was performed on days 7, 10 and 14, and half of the animals were then tracked for a further 14-21 days (FIG. 32A) There was rapid clearance of tumour in groups receiving CAR19+ T cells with negligible signal by day 14, in contrast to mice receiving non-transduced T cells or PBS (P<0.001) (FIG. 32B). Interestingly, TT TCR− CAR19+ T cells mediated clearance by day 14 was superior compared to CAR19+TCR+ cells (P<0.05) based on luminescence quantification (FIG. 32C, E). Flow cytometric analysis was undertaken for GFP+ Daudi cells and CD45+CD2+ effector T cell populations harvested from bone marrow and spleen of TT TCR− CAR19+ T cell (n=4), TCR+ CAR19+ T cell (n=5), TCR+ CAR19+ T cell (n=5) and PBS (n=3) injected mice. By day 14, less than 0.01% (0.009%±0.006%) of total marrow harvested from long bones of TT TCR− CAR19+ T cell and <0.008%±0.002% of TCR+ CAR19+ T cell injected mice expressed GFP Daudi cells indicating a highly significant (>45-fold) reduction over the 0.416%±0.301% (P<0.05) and 0.336%±0.246% tumour content detected in the marrow of CAR19−TCR+ and PBS control animals (FIG. 32D)

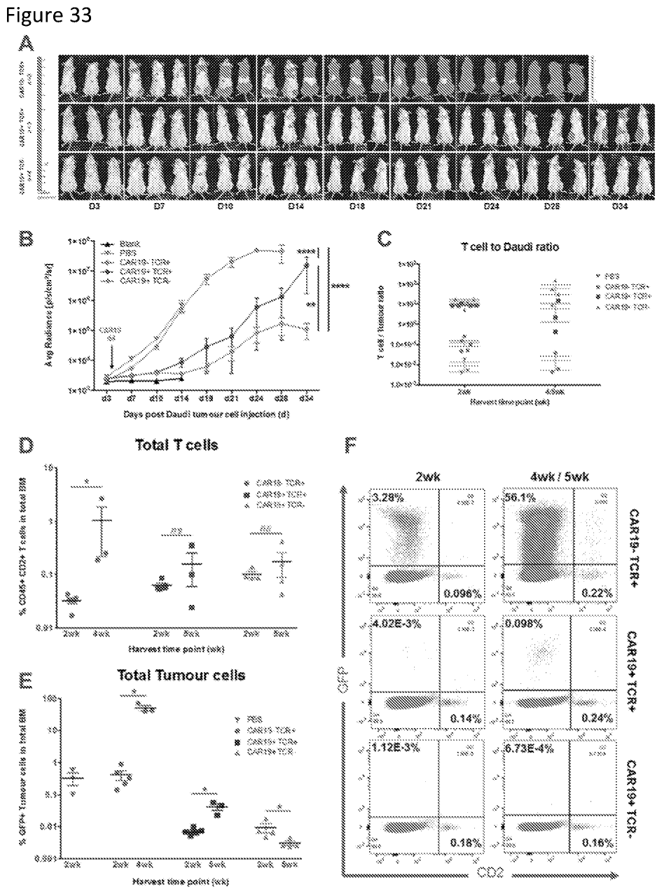

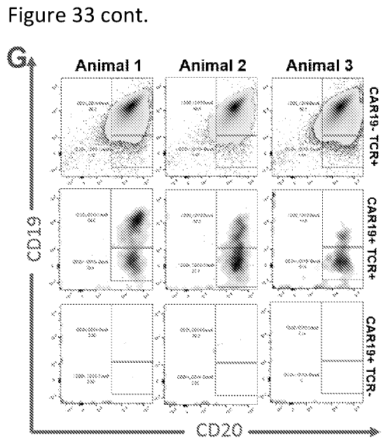

By day 28, the control CAR19−TCR+ cohort had exhibited a further 125-fold increase tumour burden and the mice were culled, revealing a low T cell to tumour ratio on flow analysis, despite a significant expansion of CD45+CD2+ T cells in the final two weeks. The remaining treatment groups were monitored until day 35 (FIG. 33A). Radiance increased in the TCR+ CAR19+ group but not the TT TCR− CAR19+ group (FIG. 33B) during this period with luminescence signal appearing to localise in the bone marrow and this was supported by flow cytometry data when GFP+ Daudi cells in marrow had increased six fold in the TCR+ CAR19+ group (P<0.05) (FIG. 33E, F). FIG. 33D shows the percentage of CD45+CD2+ T cells in total marrow for each group. Interestingly, on further analysis, we found evidence of antigen escape with clearly demarcated populations of CD20+ GFP+ Daudi cells that had lost CD19+ expression in all three animals (FIG. 33G).

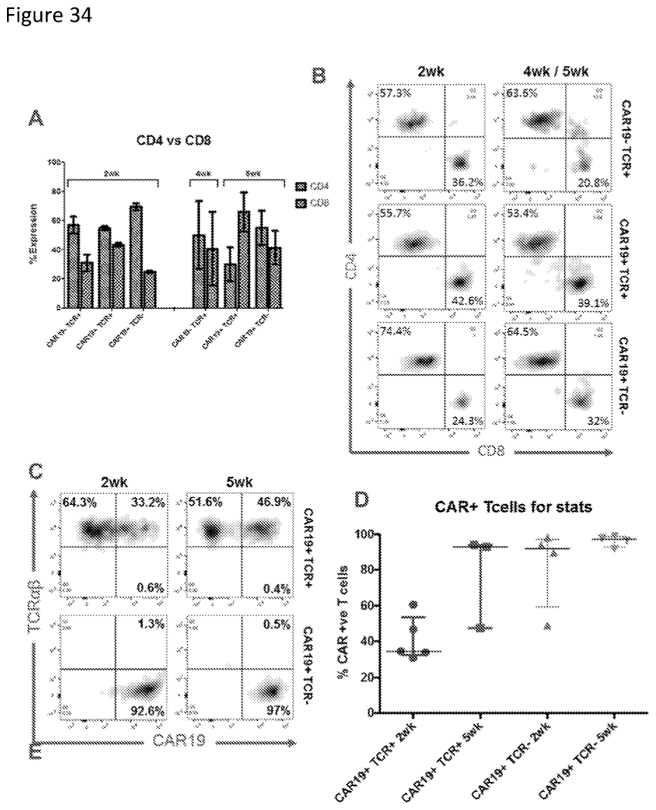

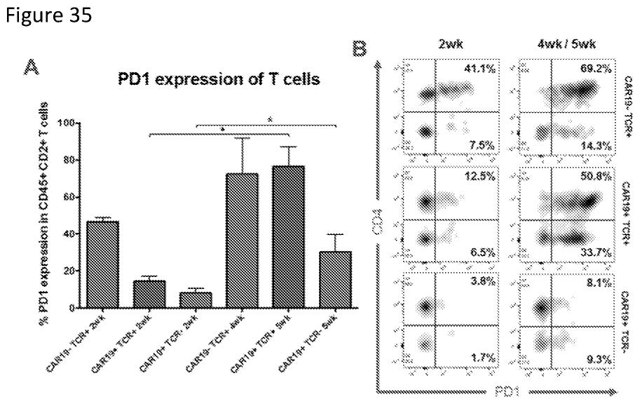

The TT TCR− CAR19+ group exhibited the highest levels of CAR19 expression at in CD4 T cells at both 2 and 5 weeks (FIG. 34A, B, C) and had the lowest tumour burden (FIG. 33E) and retained the highest T cell:tumour ratio throughout (FIG. 33C). In contrast, median CAR expression was 35% in TCR+ CAR19+ treated mice at 2 weeks and rose to 93% by 5 weeks consistent with notable expansion of transduced populations (FIG. 34D). Interestingly, these animals also exhibited high levels (76.63%±18.41%) of the exhaustion marker PD-1 on T cells compared to 14.46%±6.18% at two weeks (FIG. 35A,B). This was also documented in non-transduced CAR19−TCR+ controls where PD1 levels of 46.65%±5.17% at 2 weeks and 72.48%±33.46% at 4 weeks had been observed in CD4+ T cells. Of note, TT TCR− CAR19+ T cell effectors had least exhausted phenotype with the lowest levels of PD1 expression at both time points.

### Discussion

There is accumulating evidence for the efficacy of T cells engineered to express chimeric antigen receptors (CARs) against leukemia antigens such as CD19 in the management of relapsed B cell malignancies. As well as autologous T cells, HLA-matched allogeneic T cells from stem cell donors have been used and recently non-HLA matched ‘universal’ CAR-T cells have entered clinical phase assessments. These cells were edited using TALENs to disrupt the TRAC locus to prevent GVHD and at the CD52 locus to confer resistance to the lymphodepleting antibody Alemtuzumab. The modification process employed a multiplex approach, delivering mRNA encoding two highly specific TALEN pairs by electroporation which confers high frequency allele modification. In the case of the TRAC locus, in most T cells allelic exclusions ensures that only a single TCR configuration is expressed, and scission and NHEJ of this single allele is sufficient to disrupt cell surface TCRab expression. Downstream processing using CliniMacs TCRab magnetic bead depletion ensures removal of residual TCRab+ cells and usually yields highly purified (>99%) TCRab− T cells. One shortcoming of current approaches is variable lentiviral transduction efficiencies between batches, and as a result different total cell dosing to ensure specific CAR19 dosing. The total T cell dose is a critical and limiting factor given the restrictions placed on residual TCRab carriage, as these cells may or may not be CAR+.

We report in this Example that coupling of gene editing effects by incorporation of CRISPR sequences within the CAR19 vector configuration allows the production of TT TCR− CAR19+ T cells that are highly purified, both for CAR19 and depletion of TCRab. This system has a number of advantages. Firstly, both the transduction and TRAC editing effects are highly efficient, and secondly after the completion of downstream processing and depletion of residual TCRαβ cells, the product is highly homogeneous. Third, a single Cas9 mRNA batch will service multiple terminal vector-CRISPR RNA guide combinations, reducing costs and saving time associated with the manufacture of bespoke raw materials. Fourth, the process has been quickly incorporated into an automated manufacturing platform facilitating early clinical application. Finally, in vivo modelling has revealed that TT TCR− CAR19+ T cells mediated superior leukemic eradication with less evidence of exhaustion compared to conventionally modified cells.

The vector design exploited a key duplication effects that arise during retroviral reverse transcription, enhancing CRISPR guide RNA expression without interfering with CAR19 transgene expression. Transient expression of Cas9 following Cas9 mRNA delivery by electroporation was considered critical for time-limited DNA cleavage effects and minimizing risk of immunogenicity. We demonstrate that this lentiviral configuration supports high titre vector production and mediates sustained transgene expression.

Characterization of TCR− CAR19+ populations included flow cytometry, cytokine array profiles and functional studies in vitro ahead of in vivo anti-tumour studies. Comparisons with TCR+ CAR19+ T cells revealed superior anti-leukemic effects with an absence of xenoreactive GVHD effects, and less upregulation of exhaustion marker PD1 than control groups that retained TCR expression.

Interestingly, flow characterization of Daudi cell surface marking in mice treated with TCR+ CAR19+ T cells revealed a notable proportion had clearly lost CD19 expression, and this was not evident in tumor cells recovered from CAR-TCR+ treated mice. This phenomenon is reminiscent of clinical reports from patients who relapsed with CD19 negative leukemia after a period of remission following CAR19 therapy and we speculate that CD19 tumor populations acquired a survival advantage and expanded in the face of CAR19 immunity. Surprisingly, similar expanses were absent in TT TCR− CAR19+ T cell treated mice.

The vectors have scope to include multiple guide cassettes for multiplex modifications, including B₂M disruption to deplete MEW class I expression, CD52 to confer resistance to Alemtuzumab and PD-1 or LAG3 disruption to promote T cell invigoration.

The terminal vector configuration described here utilised Streptococcus pyogenes Cas9 but could be readily adapted for other similar nucleases, nickases, dead Cas systems, or cytidine deamination linked enzymes delivered in mRNA or protein form.

### Example 6—Generation and Efficacy of TTCAR20 (TRAC) & TBCAR20 (B2M) Peripheral Blood T Cells

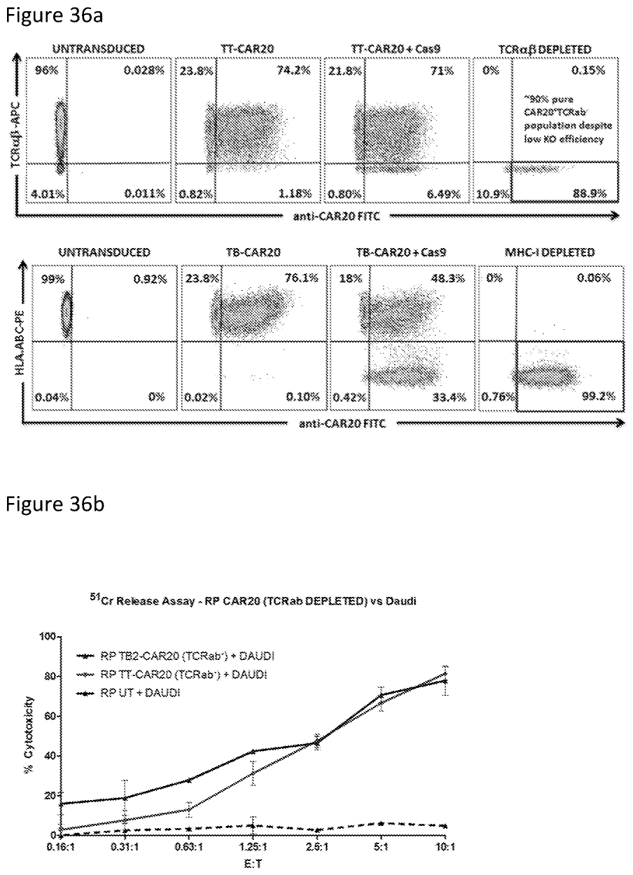

Freshly isolated PBMC were activated and transduced with TTCAR20, encoding CRISPR guide against TRAC and TBCAR20, encoding B2M specific guide for HLA-class I ko.Analysis by flow cytometry revealed 74.2% and 76.1% CAR expression respectively (FIG. 36a). Cas9 mRNA electroporation resulted in high level TRAC and HLA-ABC (MHC-I) reduction. Coupled transduction and ko effects, and TCRab or pan-HLA class I magnetic bead depletion yielded >88% TT-CAR20+TCR− and >99% TB-CAR20 T cell populations. The function of TT and TBCAR20 effectors against 51Cr labelled Daudi B-cell was assessed at different effector:target ratios. As shown in FIG. 36b, cytotoxic killing of targets across at different effector:target ratios was observed for TT and TBCAR20 effectors compared to untransduced (UT) cells.

### Example 7—Scalability of TTCAR20 Production

The scalability of universal TTCAR20 cell manufacture using the semi-automated GMP Prodigy platform was investigated.

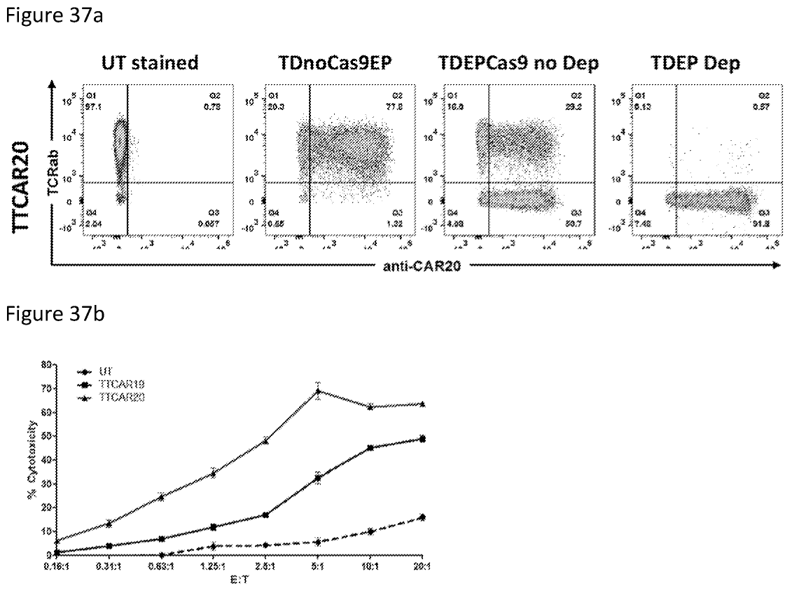

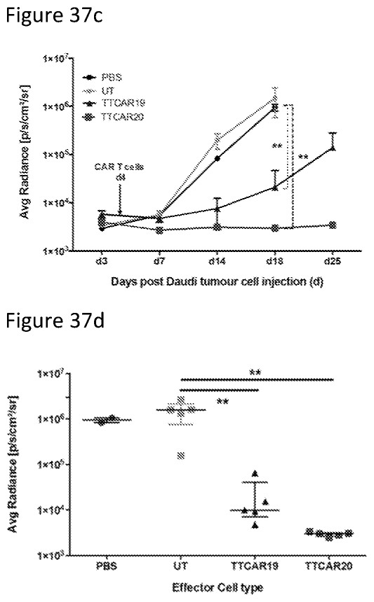

Frozen PBMCs were thawed, activated and transduced with a terminal vector coupling CRISPR-mediated TRAC knockout with anti-CD20CAR expression. As shown in FIG. 37a, transduction with TTCAR20 vector gave 77.8% CAR expression. Cas9 mRNA electroporation resulted in high level TCRab disruption. TCRab magnetic bead depletion yielded >91% TTCAR20+TCR− T cell population with 0.7% TCR+ cells carriage.

The function of TTCAR20 effectors against 51Cr labeled Daudi B-cell line was assessed at different effector:target ratios (FIG. 37b). Untransduced effectors used as negative controls showed low level cytotoxicity at higher effector:target ratios compared to TTCAR20 and TTCAR19 cells.

Immunodeficient NSG mice were inoculated intravenously with eGFP/Luciferase expressing B-cell Daudi tumours (0.5×10⁶) on day 0. Bioluminescent imaging performed on day 3 confirmed tumour engraftment. On day 4 5×10⁶ untransduced T cells (n=5), 5×10⁶ Prodigy manufactured and TCR depleted TTCAR19 T cells (n=5), 5×10⁶ Prodigy manufacture and TCR depleted TTCAR20 T cells (n=5), or PBS (n=2) were administered via tail vein injection. Bioluminescent monitoring for tumour expression was performed on days 7, 14, 18 and 25 post tumour injection. Clearance of tumour was seen following injection of TTCAR20 T cells and TTCAR19 T cells over untransduced or PBS groups (FIG. 37c).

Bioluminescent imaging performed on day 18 showed tumour progression in untransduced T cell groups to be highly significant over both TTCAR19 and TTCAR20 groups (FIG. 37d).

### Example 8—TT and TB UACT Cell Manufacture

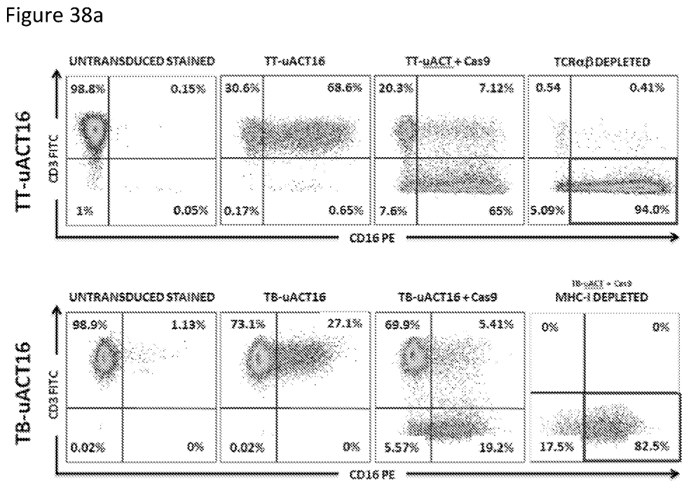

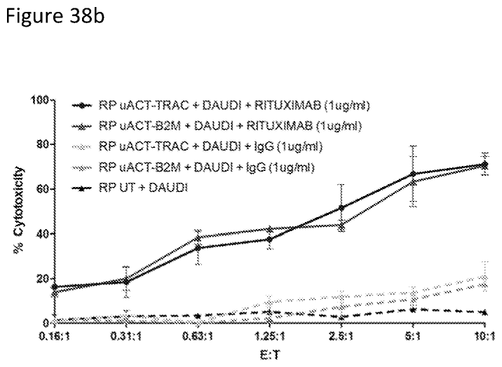

Freshly isolated PBMC were activated and transduced with a terminal vector coupling CRISPR-mediated TRAC or B2M knockout with uACT16 (FCgRIIIa) expression. Efficient transduction of primary PBMC, transduced with TTuACT16 and TB-uACT vectors showed 68.6% and 27.1% CAR expression respectively (FIG. 38a). Cas9 mRNA electroporation resulted in high level TRAC and MHC-I disruption seen by knockout in TCRab and HLA-ABC expression respectively. TCRab magnetic bead depletion yielded a 94% TTuACT16+TCR− with <1% TCR+ cells remaining and >82% MHC-I T cell population with <1% MHC-I+ cells remaining.

The function depleted uACT16 effectors against rituximab opsonized 51Cr labeled Daudi B-cell line was assessed. As shown in FIG. 28b, a high level of cytotoxic mediated killing of targets was seen across different effector:target ratios. uACT16 depleted T cells against IgG opsonized targets or untransduced cells against non-opsonised targets were used in parallel as negative controls.

### Example 9—Universal TT and TB CAR123 Manufacture

Terminal CRISPR vector was used to couple CRISPR-mediated TRAC knockout with anti-CD123 CAR expression. As shown in FIG. 329a, efficient transduction of PBMCs with TTCAR123 vector was observed. Cas9 mRNA electroporation resulted in knockout of TCR/CD3 expression. Coupling was confirmed by restricted disruption of TCR/CD3 within the transduced population. TCRab magnetic bead depletion yielded a >92% TTCAR123+TCR− T cell population with <0.5% residual TCR+ cells remaining.

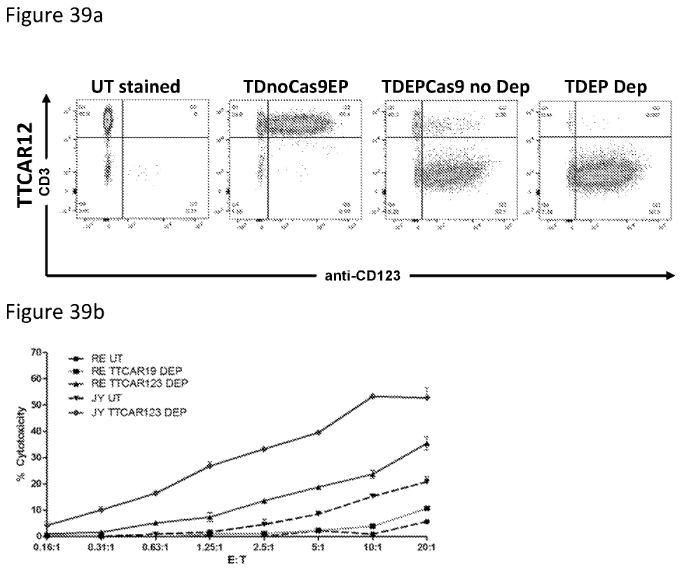

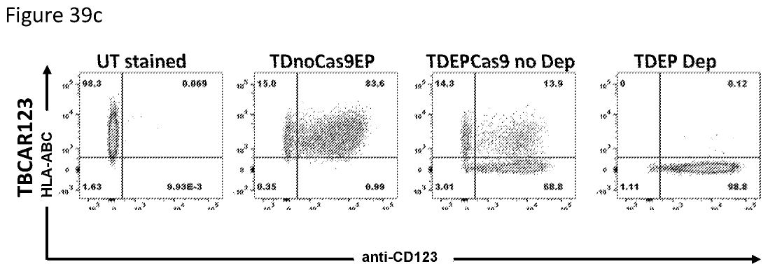

The function of depleted TTCAR123 effectors against 51Cr labeled acute myeloid leukaemia (AML) MOLM-14 cell line was assessed. As shown in FIG. 39b, TTCAR123 effectors exhibited cytotoxic mediated killing of targets. The assay was repeated using effectors from two separate donors. Untransduced or TTCAR19 transduced effectors were used as negative controls.

TBCAR123 cells were also manufactured. Terminal CRISPR vector was used to couple CRISPR-mediated B2M knockout with anti-CD123 CAR expression. As shown in FIG. 39c, efficient transduction of primary PBMCs with TBCAR123 vector was observed. Cas9 mRNA electroporation resulted in high level B2M disruption seen by knockout of HLA-A,B,C expression. Coupling was confirmed by restricted disruption within the transduced population. MHC class I magnetic bead depletion yielded a >98% TBCAR123+B2M- T cell population.

### Example 10—Universal TTCAR22 and TBCAR22 Manufacture

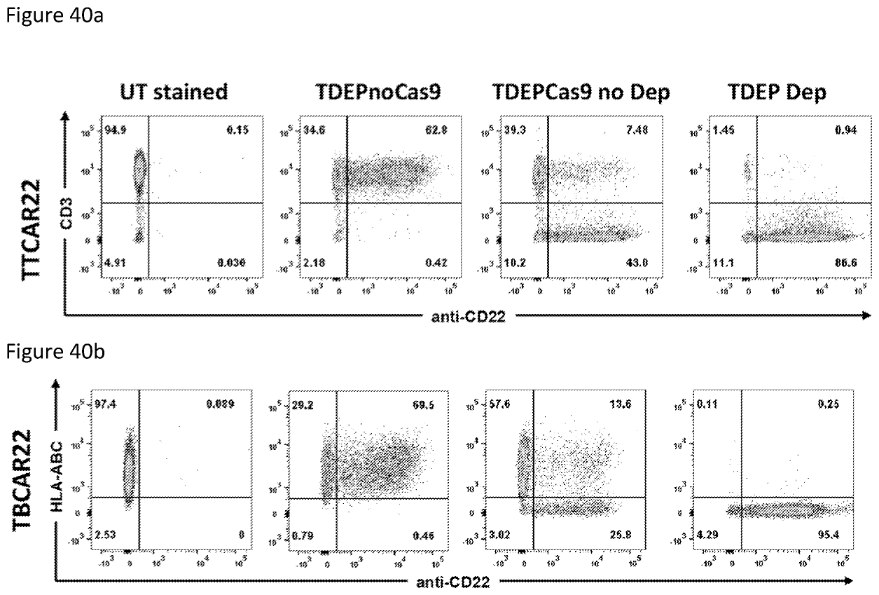

A terminal CRISPR vector was used to couple CRISPR-mediated TRAC knockout with anti-CD22 CAR expression. Efficient transduction of primary PBMCs with TTCAR22 vector was observed (FIG. 40a). Cas9 mRNA electroporation resulted in high level TRAC disruption seen by knockout of CD3 expression. Coupling was confirmed by restricted disruption within the transduced population. TCRab magnetic bead depletion yielded a >86% TTCAR22+TCR− T cell population.

A terminal CRISPR vector was also used to couple CRISPR-mediated B2M knockout with anti-CD22CAR expression. Efficient transduction of primary PBMCs TBCAR22 vector was observed (FIG. 40b). Cas9 mRNA electroporation resulted in high level B2M disruption seen by knockout of HLA-A,B,C expression. Coupling was confirmed by restricted disruption within the transduced population. MHC class I magnetic bead depletion yielded a >95% TBCAR22+B2M- T cell population.

### Example 11—Universal TTCAR20 Cord T Cell Manufacture

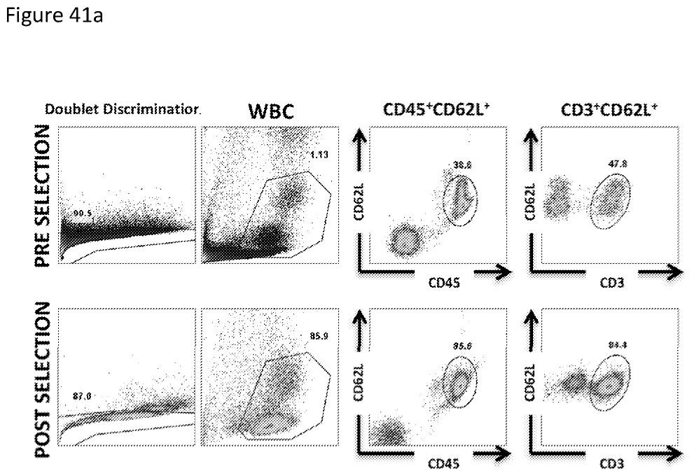

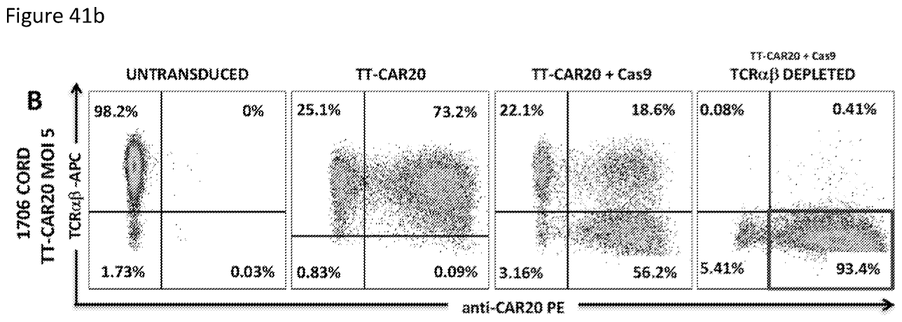

Cord blood cells were enriched for CD62L populations using semi-automated GMP Prodigy platform. The selection steps and resultant populations are shown in FIG. 41a.

CD62L+ cord blood cells were activated and transduced with a terminal vector coupling CRISPR-mediated TRAC knockout with CAR20 expression (FIG. 41b). Efficient transduction of CD62L enriched cord blood cells, transduced with the TT-CAR20 vector showed 73.2% CAR expression respectively. Cas9 mRNA electroporation resulted in high level TRAC disruption seen by knockout in TCRab expression. Coupling was confirmed by restricted KO solely within the CAR20 transduced populations. TCRab magnetic bead depletion yielded a >93.4% TT-CAR20+TCR− T-cell populations with <1% TCR+ cells remaining.

### Example 12—TT-UACT Cord T Cell Manufacture

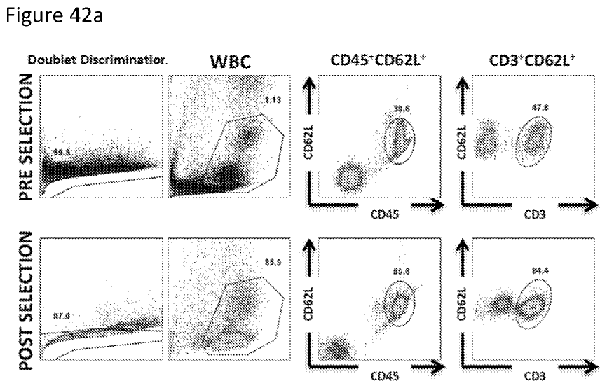

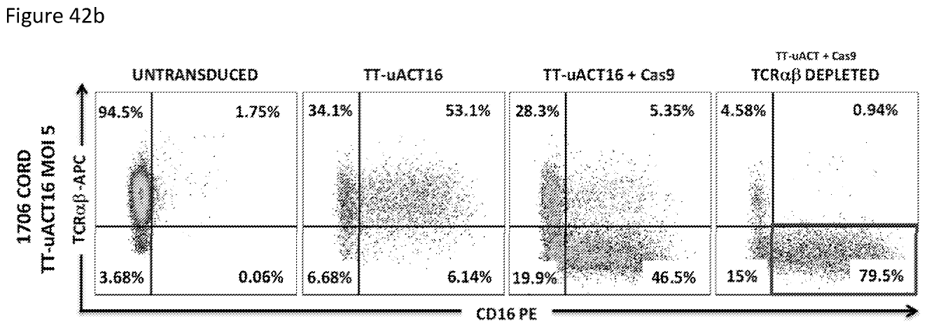

Cord blood cells were enriched for CD62L populations using semi-automated GMP Prodigy platform. The selection steps and resultant populations are shown in FIG. 42a.

CD62L+ cord blood cells were activated and transduced with a terminal vector coupling CRISPR-mediated TRAC knockout with CD16 (FCγRIIIa) expression (FIG. 42b). Efficient transduction of CD62L enriched cord blood cells, transduced with the TT-uACT16 vector showed 53.1% CAR expression respectively. Cas9 mRNA electroporation resulted in high level TRAC disruption seen by knockout in TCRab expression. TCRab magnetic bead depletion yielded a >79% TT-uACT16+TCR− T-cell populations

### Example 13—Multiplexing Terminal CRISPR

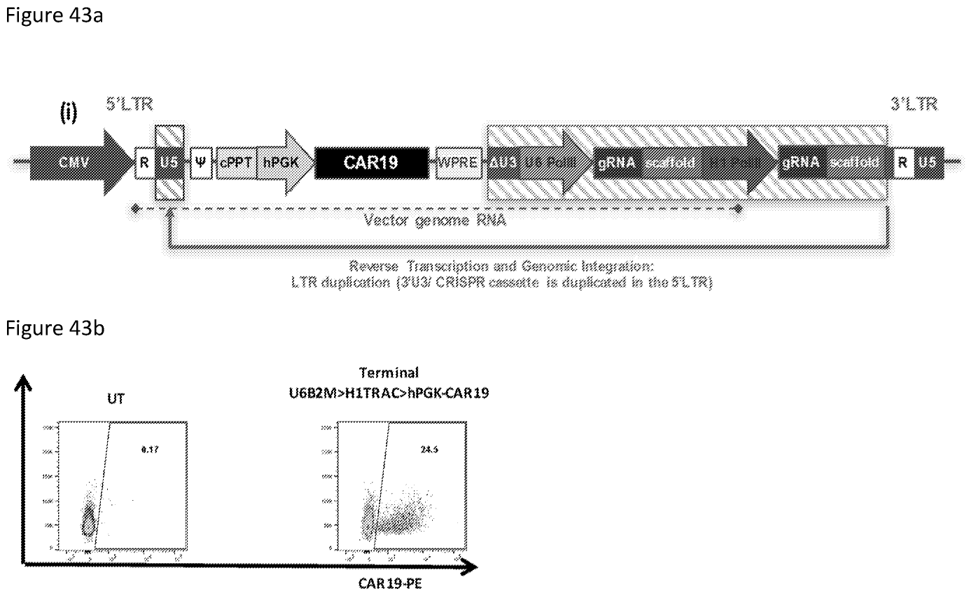

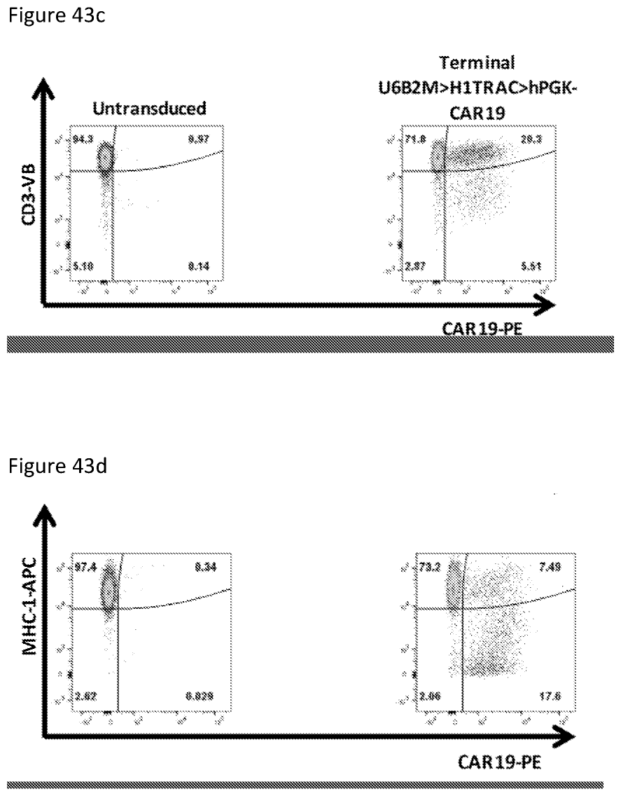

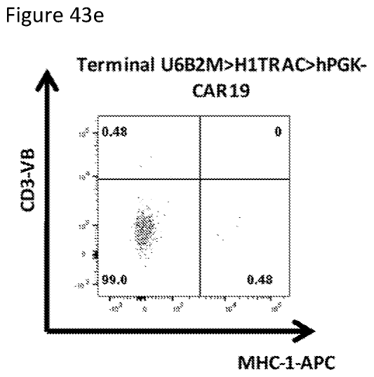

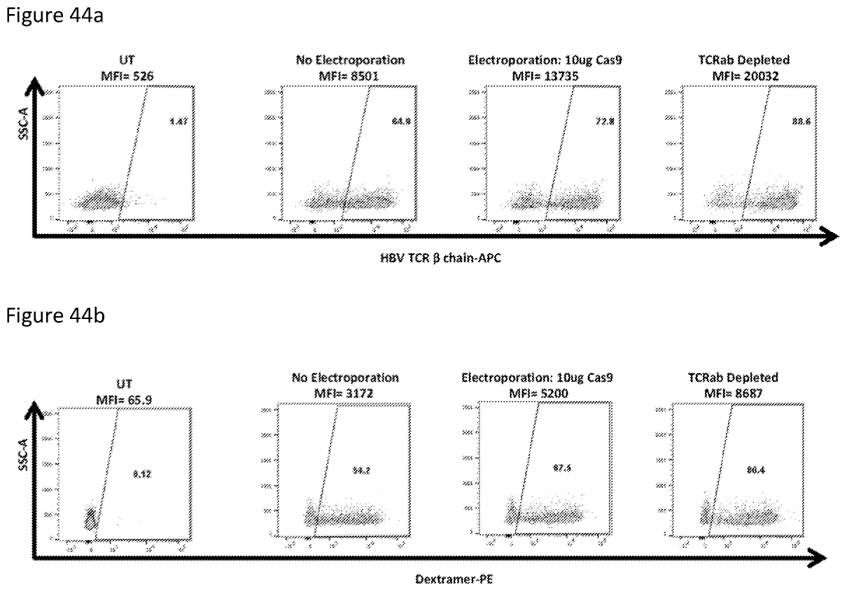

An exemplary terminal vector multiplex configurations supporting expression of multiple sgRNAs is shown in FIG. 43a. In this example, there are two CRISPR guide cassettes within the ΔU3 region of the 3′LTR under the control of distinct promoters such as U6, full length or minimal H1 Pol III promoters to avoid recombination effects. Vector titres were sustained above 10⁸/ml after ultracentrifugation. While sgRNA for TRAC and B2M are shown in FIG. 43a, other dual combinations are possible. Exemplary dual combinations may target TRAC and CD52, TRAC and PD1, PD1 and B2M, TRAC and CD123, or TRAC and CD52.

Primary human T cells were transduced with multiplex terminal CRISPR vector after activation with Transact at an MOI of 5, with a transduction efficiency of −25% (FIG. 43b). Cas9 mRNA was electroporated. FIGS. 43c and 43d show simultaneous disruption of TRAC (via CD3 staining shown in FIG. 43c) and β2M (via HLA class I staining shown in FIG. 43d) seven days after electroporation.

Biotinylated bead conjugated antibodies against TCR and HLA-class I were used to perform combined depletion of residual TCRab T cells and HLA class I expressing cells. The final product is 99% TCR− HLA class I—after a single round of depletion (FIG. 43e). Such a product could be used in HLA mismatched settings without risk of GVHD or T cell mediated rejection.

### Example 14—TCR Devoid rTCR Engineered T Cells

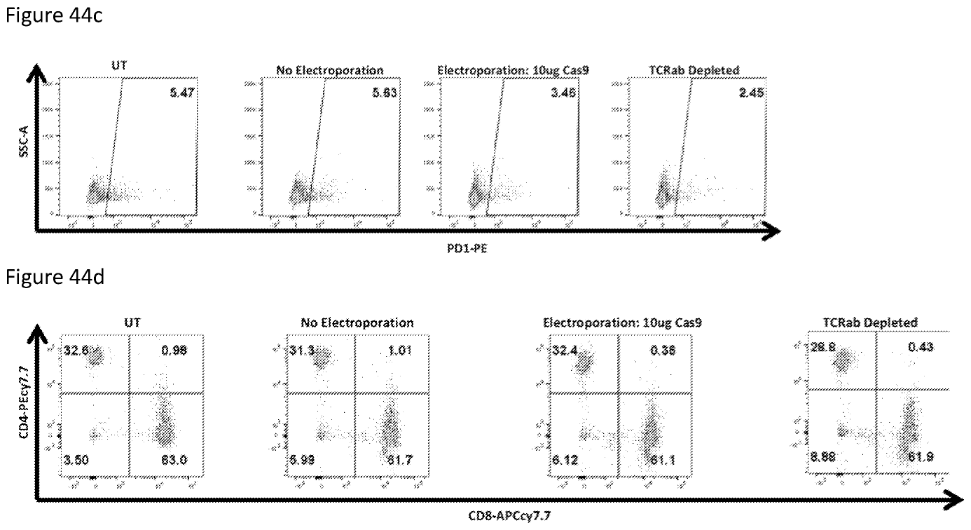

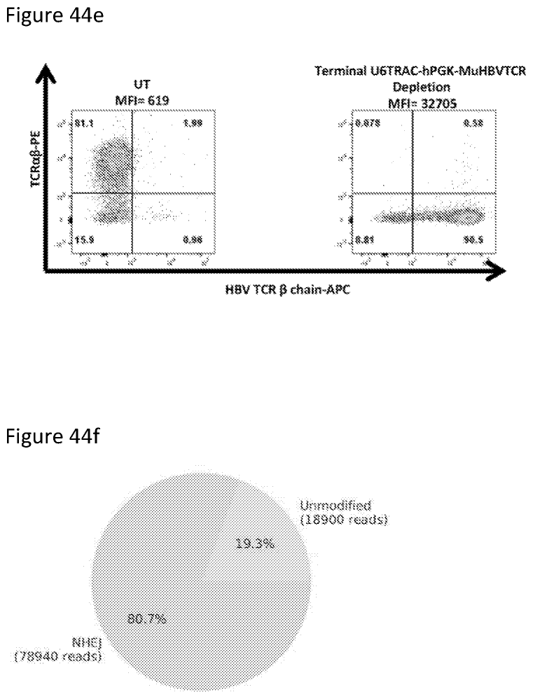

Recombinant HBV TCRab was delivered using TT lentiviral terminal CRISPR vector. Delivery of HBV TCRab was measured using VB2 antibody staining for the chain of this transgenic TCR (FIG. 44a). HBV TCRab expression was enhanced after depletion of endogenous TCR by TRAC disruption following Cas9 electroporation. As shown by dextramer staining in FIG. 44b, competition from endogenous TCRab expression was abrogated

The phenotype of rTCR engineered T cells was monitored throughout production, and showed low expression of PD1, an early exhaustion marker (FIG. 44c). In particular, PD1 was expressed on less than 3% of cells after culture and TCRαβ depletion. CD4 and CD8 expression was unchanged through production (FIG. 44d).

As shown in FIG. 44e, <1% carriage of endogenous TCR and 90% rTCR expression was observed after depletion of residual TCRab T cells using magnetic beads, as a result of differential epitope expression. Next generation sequencing confirmed 80% of TRAC alleles were modified by NHEJ, consistent with depletion of >99% TCR expressing cells given that allelic exclusion operates at the TRAC locus (FIG. 34f).

### Example 15—Base Editing Delivered by TTCAR19 Vector to Disrupt TRAC without DNA Cleavage

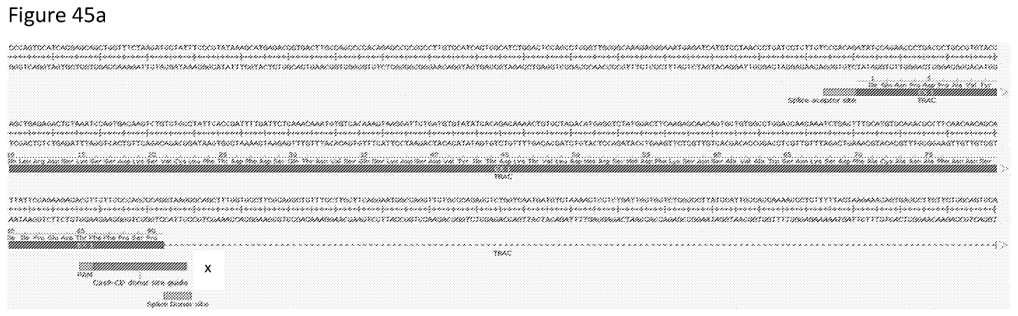

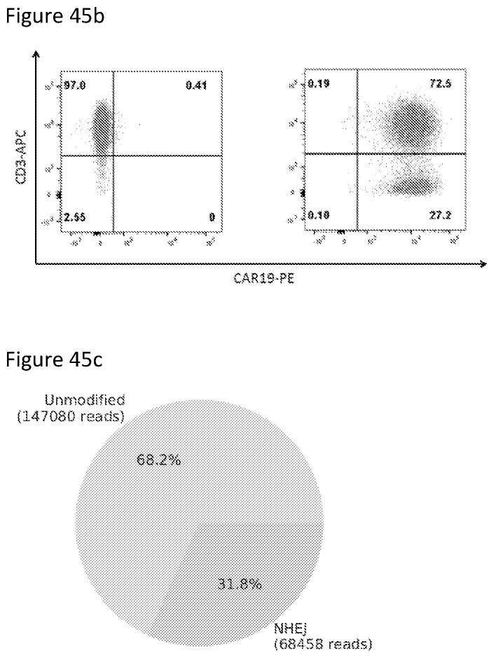

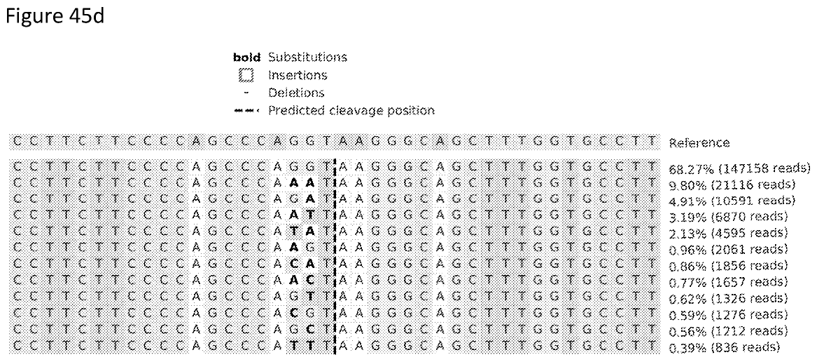

FIG. 45a shows the DNA sequence of exon 1 of TRAC showing sgRNA target region (x) for cytidine deaminase base editor (BE3). The sgRNA was designed to specifically create modifications within the exon 1 splice donor site.

T cells were transduced to express BE3 and then treated with TTCAR19 expressing the Ex1 sgRNA (shown in FIG. 45a). This resulted in ˜27% TCR/disruption, as assessed using flow cytometry (FIG. 45b). Next generation sequencing found signatures of editing of around 30% (FIG. 45c). These were mostly substitutions rather than Indels. As shown in FIG. 45d, TTCAR19/BE3 mediated predominantly C>T/G>A substitutions of base pairs 4-8 proximal of the PAM, resulting in loss of integrity of the splice donor site.

### Further Embodiments of the Invention

1. A method for generating universal antibody dependent cord T cells (U-ACTs), comprising:
   (a) providing a sample of cord blood;
   (b) separating cells that express CD62L from the sample, wherein the cells that express CD62L comprise cord blood T cells;
   (c) introducing into one or more of said cord blood T cells of (b) a nucleic acid sequence encoding an Fc-Receptor (FcR) that comprises (I) an extracellular domain that is capable of binding to a constant domain of an antibody and (II) a transmembrane domain and a cytoplasmic domain that are capable of supporting T cell activation; and
   (d) disrupting expression of T cell receptor and WIC class I in said cord blood T cells of (c),
   wherein, in (d), the expression of T cell receptor and/or MHC class 1 is disrupted by:
   (i) introducing one or more CRISPR guide sequences to said cord blood T cells of
      (c) using a vector that comprises a 3′ long terminal repeat region (LTR) comprising one or more promoter sequences operably linked to the sequence encoding said CRISPR guide sequence(s); and
   ii) separately delivering a CRISPR nuclease to said cord blood T cells of (c) by introducing into them a nucleic acid sequence encoding said CRISPR nuclease.
2. A method for generating universal antibody dependent cord T cells (U-ACTs), comprising:
   (a) providing one or more cord blood T cells;
   (b) introducing into one or more of said cord blood T cells of (a) a nucleic acid sequence encoding an Fc-Receptor (FcR) that (I) an extracellular domain that is capable of binding to a constant domain of an antibody and (II) a transmembrane domain and a cytoplasmic domain that are capable of supporting T cell activation; and
   (c) disrupting expression of T cell receptor and MHC class I in said cord blood T cells of (b).
3. A method for generating cord blood T cells, comprising:
   (a) providing a sample of cord blood; and
   (b) separating cells that express CD62L from the sample wherein the cells that express CD62L comprise one or more cord blood T cells.
4. A method for delivering CRISPR guide sequences and a CRISPR nuclease to a cell, comprising:
   (a) introducing one or more CRISPR guide sequences to said cell using a vector that comprises a 3′ long terminal repeat region (LTR) comprising one or more promoter sequences operably linked to the sequence encoding said CRISPR guide sequence(s); and
   (b) separately delivering the CRISPR nuclease to said cell of (a) by introducing into it a nucleic acid sequence encoding said CRISPR nuclease.
5. The method of item 1 or 2, wherein the extracellular domain comprises an extracellular domain of a variant FcRIIIA
6. The method of any one of items 1, 2 or 5, wherein the antibody is an IgG antibody.
7. The method of item 6, wherein the antibody is an IgG1 antibody.
8. The method of any one of items 1, 2, or 5 to 7, wherein the antibody is a monoclonal antibody.
9. The method of item 8, wherein the monoclonal antibody is a therapeutic antibody.
10. The method of any one of items 1, 2 or 5 to 9, wherein the antibody is specific for a tumour antigen.
11. The method of any one of items 1, 2 or 5 to 10, wherein the antibody is specific for a B-cell antigen.
12. The method of any one of items 1, 2 or 5 to 10, wherein the antibody is specific for CD20, CD22, CD38, CD52, EGFR, ERB2/HER2, CD30, GD2 or VEGFR.
13. The method of item 12, wherein the antibody is Rituximab, Ofatumumab, Inotuzumab, Dartumumab, Alemtuzumab, Panitumumab, Herceptin, Pertuzumab, Brentuximab vedotin, Dinutuximab, or Tamucirumab.
14. The method of any one of items 1, 2 or 5 to 13, wherein the cytoplasmic domain comprises a 41BB activation domain.
15. The method of any one of items 1, 2 or 5 to 14, wherein the cytoplasmic domain comprises a CD3 activation domain.
16. The method of any one of items 1, 2 or 5 to 15, wherein the transmembrane domain comprises a CD8 transmembrane domain.
17. The method of any one of items 1, 2 or 5 to 16, wherein the FcR comprises a spacer.
18. The method of item 17, wherein the spacer is an immunoglobulin light chain variable region.
19. The method of item 18, wherein the immunoglobulin light chain variable region facilitates FcR dimerization.
20. The method of any one of items 1, 2 or 5 to 19, wherein the cytoplasmic domain comprises a CD3e activation domain
21. The method of any one of items 1, 2 and 5 to 20, wherein the nucleic acid sequence encoding the FcR is delivered to the cell using a viral vector.
22. The method of item 21, wherein the viral vector is a lentiviral vector.
23. The method of any one of items 1, 2 and 5 to 22, wherein the expression of T cell receptor or MHC class 1 is disrupted using TALENs, meganucleases, ZFNs or CRISPR.
24. The method of any one of items 1, 2 and 5 to 23, wherein the expression of T cell receptor is disrupted by targeting the TRAC locus, TCR beta constant locus or CD3 locus.
25. The method of any one of items 1, 2 and 5 to 24, wherein the expression of MHC class 1 is disrupted by targeting the TAP1, TAP2, CIITA, RFX5, RFXAP or RFXANK or β₂m locus.
26. The method of item 2, wherein the one or more cord blood T cells are generated using the method of item 3.
27. The method of item 2, wherein the expression of T cell receptor and/or MHC class I is disrupted by delivering CRISPR guide sequences and a CRISPR nuclease to the one or more cord blood T cells using the method of item 4.
28. The method of item 1 or 3, wherein the cells that express CD62L are separated from the sample based on their ability to bind an anti-CD62L antibody.
29. The method of item 28, wherein magnetic-activated cell sorting (MACS) is used to separate the cells that express CD62L from the sample.
30. The method of item 28, wherein fluorescence-activated cell sorting (FACS) is used to separate the cells that express CD62L from the sample.
31. The method of any one of items 1, 3 or 28 to 30, wherein step (b) is performed in a system configured for automated production of cord T cells.
32. The method of any one of items 1, 3 and 28 to 30, wherein after step (b) the cells that express CD62L are stimulated using an anti-CD3 antibody and/or an anti-CD28 antibody.
33. The method of item 3, wherein after step (b) a nucleic acid sequence encoding an FcR according to item 1 or a chimeric antigen receptor (CAR) is introduced into one or more of said cells that express CD62L.
34. The method of item 33, wherein the CAR specifically binds to CD19, CD20, CD22, CD33, CD123, CD30, erb-B2, CEA, IL13R, Ror, kappa light chain, TCR-beta constant 1, TCR-beta constant 2, MAGE-A1, MUC1, PSMA, VEGF-R, Her2, CAIX, CD7, CD45 or CD3.
35. The method of item 1, wherein the CRISPR guide sequences and the nucleic acid sequence encoding the FcR are delivered to the cell using the same vector.
36. The method of item 35, wherein the vector is a viral vector, preferably a lentiviral vector.
37. The method of any one of items 1, 4, 35 or 36, wherein the LTR comprises one or more H1 promoter sequences.
38. The method of any one of items 1, 4 or 35 to 37, wherein the LTR comprises one or more U6 promoter sequences.
39. The method of any one of items 1, 4 or 35 to 38, wherein following delivery of the vector, the promoter sequence is duplicated during reverse transcription such that it becomes incorporated into both the 5′ and 3′ LTRs.
40. The method of any one of items 1, 4, or 35 to 39, wherein the CRISPR nuclease is Cas9.
41. The method of any one of items 1, 4, or 35 to 40, wherein the nucleic acid sequence encoding said CRISPR nuclease is an RNA sequence.
42. The method of any one of items 1, 4, or 35 to 41, wherein the viral vector further comprises a sequence encoding a CAR.
43. The method of item 42, wherein the CAR is specific for CD2, CD3, CD7, CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX.
44. The method of any one of items 1, 4 or 35 to 43, wherein:
   (a) one or more of the CRISPR guide sequences is specific for the TRAC locus, TCR beta constant locus or CD3 locus;
   (b) one or more of the CRISPR guide sequences is specific for the β₂m, TAP1, TAP2, CIITA, RFX5, RFXAP or RFXANK locus;
   (c) one or more of the CRISPR guide sequences is specific for a locus controlling a checkpoint inhibitor pathway;
   (d) one or more of the CRISPR guide sequences is specific for the locus controlling expression of CD52; and/or
   (e) one or more of the CRISPR guide sequences is specific for a locus controlling the expression of an antigen targeted by a CAR, chimeric FcR or monoclonal antibody expressed by the cell(s).
45. The method of any one of items 4 or 35 to 44, wherein the cell is a cord blood T cell.
46. The method of item 45, wherein the cord blood T cell is generated by the method of item 3.
47. The method of any one of items 4 or 35 to 44, wherein the cell is a peripheral blood lymphocyte.
48. The method of any one of items 4 or 35 to 44, wherein the cell is a hematopoietic stem cell.
49. The method of any one of items 4, 35 to 44, 47 or 48, wherein the cell is allogeneic to an individual into which it is to be administered.
50. The method of any one of items 4, 35 to 44, 47 or 48, wherein the cell is autologous to an individual into which it is to be administered.
51. A FcR that comprises (I) an extracellular domain that is capable of binding to a constant domain of an antibody and (II) a transmembrane domain and a cytoplasmic domain that are capable of supporting T cell activation.
52. The FcR of item 51, wherein:
   (a) the extracellular domain is as defined in item 5;
   (b) the antibody is as defined in any one of items 6 to 13;
   (c) the cytoplasmic domain is as defined in item 14 or 15;
   (d) the transmembrane domain is as defined in item 16; and/or
   (e) the FcR comprises a spacer, optionally wherein the spacer is as defined in item 18 or 19.
53. A dimer comprising two FcRs according to item 51 or 52.
54. A nucleic acid sequence encoding a FcR according to item 51 or 52.
55. A vector comprising the nucleic acid according to item 54.
56. A cell comprising the nucleic acid according to item 54 or the vector according to item 55.
57. A vector that comprises a 3′ LTR comprising one or more promoter sequences operably linked to a sequence encoding one or more CRISPR guide sequences.
58. The vector according to item 57, wherein the LTR comprises one or more H1 promoter sequences.
59. The vector according to item 57 or 58, wherein the LTR comprises one or more U6 promoter sequences.
60. The vector according to any one of items 57 to 59, further comprising a nucleic acid sequence that encodes a FcR according to any one of items 51 or 52.
61. The vector according to any one of items 57 to 60, further comprising a nucleic sequence that encodes a CAR.
62. The vector according to item 61, wherein the CAR is specific for CD2, CD3, CD7, CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX
63. The vector according to any one of items 57 to 62, further comprising a nucleic acid sequence that encodes a recombinant TCR (rTCR).
64. The vector according to any one of items 57 to 63, further comprising a nucleic acid sequence that encodes a restriction factor.
65. The vector according to item 64, wherein the restriction factor is TRIM5CypA.
66. The vector according to any one of items 57 to 65, further comprising a nucleic acid sequence that encodes a suicide gene.
67. The vector according to any one of items 57 to 66, wherein one or more of the CRISPR guide sequences is specific for a locus controlling the expression of the TCR−CD3 complex.
68. The vector according to item 67, wherein one or more of the CRISPR guide sequences is specific for the TRAC locus, TCR beta constant locus or CD3 locus.
69. The vector according to any one of items 57 to 68, wherein one or more of the CRISPR guide sequences is specific for a locus controlling the expression of the MHC class 1.
70. The vector according to item 69, wherein one or more of the CRISPR guide sequences is specific for the TAP2, CIITA, RFX5, RFXAP or RFXANK, 13₂m or TAP1 locus.
71. The vector according to any one of items 57 to 70, wherein one or more of the CRISPR guide sequences is specific for a locus controlling a checkpoint inhibitor pathway.
72. The vector according to any one of items 57 to 71, wherein one or more of the CRISPR guide sequences is specific for a locus associated with a gain of function mutation.
73. The vector according to any one of items 57 to 72, wherein one or more of the CRISPR guide sequences is specific for a locus controlling transgene silencing pathway.
74. The vector according to any one of items 57 to 73, wherein one or more of the CRISPR guide sequences is specific for the locus controlling expression of CD52.
75. The vector according to any one off items 57 to 73, wherein one or more of the CRISPR guide sequences is specific for a locus controlling the expression of an antigen targeted by a CAR, chimeric FcR or monoclonal antibody expressed by the cell(s)
76. A universal antibody dependent cord T cell (U-ACT) that comprises a FcR according to item 51 or 52 and has disrupted T cell receptor and WIC class I expression.
77. A pharmaceutical composition comprising a U-ACT according to item 76.
78. A universal antibody dependent cord T cell (U-ACT) according to item 76, for use in method of treatment of the human or animal body.
79. A universal antibody dependent cord T cell (U-ACT) according to item 76, for use in a method of treating a neoplastic condition, an autoimmune condition, an infectious condition, an inflammatory condition, a haematological disorder, or a metabolic condition.
80. A universal antibody dependent cord T cell (U-ACT) according to item 76, for use in a method of depleting immune cells and/or bone marrow cells in an individual.
81. A universal antibody dependent cord T cell (U-ACT) for use according to item 80, wherein the method is performed prior to transplantation of an organ, tissue or cells into the individual.
82. A method of treating a neoplastic condition, an autoimmune condition, an infectious condition, a heaematological disorder, or an inflammatory condition in a patient in need thereof, the method comprising administering to the patient an effective number of U-ACTs according to item 76.
83. The method according to item 82, further comprising administering to the patient a therapeutic antibody.
84. Use of the vector according to any one of items 57 to 75 to disrupt expression of TCR and/or MEW class 1 in a cell.
85. Use of the vector according to any one of items 57 to 75 to introduce a nucleic acid sequence encoding a FcR according to item 51 or 52 into a cell.
86. Use of the vector according to any one of items 57 to 75 to introduce a nucleic acid sequence encoding a CAR into a cell.
87. Use of the vector according to any one of items 57 to 75 to introduce a nucleic acid sequence encoding a CAR into a cell and to disrupt expression of TCR and/or MHC class 1 in the cell.
88. The use of item 84 or 85, wherein the CAR is specific for CD2, CD3, CD7, CD10, CD19, CD20, CD22, CD30, CD33, CD45, CD123, erb-B2, CEA, IL13R, Ror, kappa light chain, TCR-beta constant 1, TCR-beta constant 2, MAGE-A1 MUC1, PSMA, VEGF-R, Her2, or CAIX
89. Use of the vector according to any one of items 57 to 75 to introduce a nucleic acid sequence encoding a rTCR into a cell and to disrupt expression of TCR in the cell.
90. Use of the vector according to any one of items 57 to 75 to introduce a nucleic acid sequence encoding a restriction factor into a cell and to disrupt expression of CCR5 in the cell.
91. The use of item 90, wherein the restriction factor is TRIM5CypA.
92. Use of the vector according to any one of items 57 to 75 to disrupt expression of a locus controlling a gain of function mutation in a cell and to introduce a nucleic acid sequence encoding a replacement protein into the cell.
93. Use of the vector according to any one of items 57 to 75 to disrupt expression of a locus controlling a transgene silencing pathway in a cell.
94. The use of item 93, wherein the vector comprises a nucleic acid sequence encoding a transgene silenced by the pathway.
95. Use of the vector according to any one of items 57 to 75 to disrupt expression of a locus controlling a checkpoint inhibitor pathway in a cell and to introduce a nucleic acid sequence encoding a suicide into the cell.
96. The use of any one of items 42 to 95, wherein the cell is a cord blood T cell.
97. The use of item 96, wherein the cord blood T cell is generated by the method of item 3.
98. The use of any one of items 84 to 99, wherein the cell is a peripheral blood lymphocyte.
99. The use of any one of items 84 to 99, wherein the cell is a hematopoietic stem cell.
100. The use of any one of items 84 to 99, wherein the cell is allogeneic to an individual into which it is to be administered.
101. The use of any one of items 84 to 99, wherein the cell is autologous to an individual into which it is to be administered.
102. The method of any one of items 4 or 37 to 50, wherein the viral vector comprises a sequence encoding a CAR specific for CD19.
103. The method of any one of items 4, 37 to 50, and 102, wherein one or more of the CRISPR guide sequences is specific for a locus controlling the expression of the TCR−CD3 complex.
104. The method of item 103, wherein one or more of the CRISPR guide sequences is specific for the TRAC locus.
105. The vector of any one of items 57 to 75, comprising a nucleic acid sequence encoding a CAR specific for CD19.
106. The vector of any one of items 57 to 75 and 104, wherein one or more of the CRISPR guide sequences is specific for a locus controlling the expression of the TCR−CD3 complex.
107. The vector of item 106, wherein one or more of the CRISPR guide sequences is specific for the TRAC locus.
108. A method for generating T cells that comprise a nucleic acid sequence encoding a CAR specific for CD19 and have disrupted TCR expression, comprising:
   (a) providing one or more T cells;
   (b) introducing into one or more of said T cells of (a) a nucleic acid sequence encoding a CAR specific for CD19; and
   (c) disrupting expression of TCR in said T cells of (b),
   wherein, in (c), the expression of TCR is disrupted by:
   (i) introducing one or more CRISPR guide sequences to said T cells of (b) using a vector that comprises a 3′ long terminal repeat region (LTR) comprising one or more promoter sequences operably linked to the sequence encoding said CRISPR guide sequence(s); and
   ii) separately delivering a CRISPR nuclease to said T cells of (b) by introducing into them a nucleic acid sequence encoding said CRISPR nuclease.
109. The method of item 108, wherein one or more of the CRISPR guide sequences are specific for TRAC.
110. The method of item 109 or 110, wherein the T cells of (a) are cord blood T cells.
111. The method of any one of items 108 to 110, wherein the CRISPR nuclease is Cas9.
112. A T cell that comprises a nucleic acid sequence encoding a CAR specific for CD19 and has disrupted TCR expression.
113. The T cell of item 112, produced according to the method of any one of items 106 to 109.
114. The T cell of item 112 or 113, wherein the T cell has disrupted TRAC expression.
115. The T cell of any one of items 112 to 114 for use in a method of treatment of the human or animal body.
116. The T cell of any one of items 112 to 114 for use in a method of treating a neoplastic condition, an autoimmune condition, an infectious condition, an inflammatory condition, a haematological disorder or a metabolic condition.
117. A method of treating a neoplastic condition, an autoimmune condition, an infectious condition, an inflammatory condition, a haematological disorder or a metabolic condition in a patient in need thereof, the method comprising administering to the patient an effective number of T cells according to any one of items 112 to 114.
118. The T cell for use of item 116 or the method of item 117, wherein the neoplastic condition is a cancer or tumour.
119. The T cell for use of item 118 or the method of item 118, wherein the cancer is leukaemia.
120. The T cell for use of item 119 or the method of item 119, wherein the leukaemia is acute lymphoblastic leukaemia, acute myeloid leukaemia, chronic lymphocytic leukaemia or chronic myeloid leukaemia.
121. Use of the vector according to any one of items 105 to 107 to disrupt expression of TCR in a cell.
122. Use of the vector according to any one of items 105 to 107 to disrupt expression of TRAC in a cell
123. Use of the vector according to any one of item 105 to 107 to introduce a nucleic acid sequence encoding CAR specific for CD19 into a cell.
124. Use of the vector according to any one of item 105 or 107 to introduce a nucleic acid sequence encoding CAR specific for CD19 into a cell and to disrupt expression of TCR in the cell.
125. The use of any one of items 121 to 124, wherein the cell is T cell.
126. The use of item 125, wherein the T cell is a cord blood T cell.
127. A pharmaceutical composition comprising a T cell according to any one of items 112 to 1

1. A method for delivering CRISPR guide sequences and a CRISPR guided DNA modification enzyme to a cell, comprising:
(a) introducing one or more CRISPR guide sequences to said cell using a vector that comprises a 3′ long terminal repeat region (LTR) comprising one or more promoter sequences operably linked to the sequence encoding said CRISPR guide sequence(s); and
(b) separately delivering the CRISPR guided DNA modification enzyme to said cell of (a) by introducing into it a nucleic acid or protein sequence encoding said CRISPR guided DNA modification enzyme.

2. The method of claim 1, wherein the LTR comprises a H1 promoter sequence and/or a U6 promoter sequence.

3. The method of claim 1 or 2, wherein the LTR comprises two or more sequences encoding a CRISPR guide sequence, each operably linked to a different promoter sequence.

4. The method of any one of the preceding claims, wherein the vector is a viral vector, preferably a lentiviral vector.

5. The method of any one of the preceding claims, wherein following delivery of the vector, the promoter sequence is duplicated during reverse transcription such that it becomes incorporated into both the 5′ and 3′ LTRs.

6. The method of any one of the preceding claims, wherein the CRISPR guided DNA modification enzyme is a cytidine deaminase or a CRISPR nuclease, optionally Cas9.

7. The method of any one of the preceding claims, wherein the nucleic acid sequence encoding said CRISPR guided DNA modification enzyme is an RNA sequence.

8. The method of any one of the preceding claims, wherein the vector further comprises a sequence encoding a CAR.

9. The method of claim 8, wherein the CAR is specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1 MUC1, PSMA, VEGF-R, Her2, or CAIX.

10. The method of any one of the preceding claims, wherein:
(a) one or more of the CRISPR guide sequences is specific for the TRAC locus, TCR beta constant locus or CD3 locus;
(b) one or more of the CRISPR guide sequences is specific for the β₂m, TAP1, TAP2, CIITA, RFX5, RFXAP or RFXANK locus;
(c) one or more of the CRISPR guide sequences is specific for a locus controlling a checkpoint inhibitor pathway;
(d) one or more of the CRISPR guide sequences is specific for the locus controlling expression of CD52; and/or
(e) one or more of the CRISPR guide sequences is specific for a locus controlling the expression of an antigen targeted by a CAR, chimeric FcR or monoclonal antibody expressed by the cell(s).

11. The method of any one of the preceding claims, wherein (a) the vector further comprises a sequence encoding an Fc-Receptor (FcR) that comprises (I) an extracellular domain that is capable of binding to a constant domain of an antibody and (II) a transmembrane domain and a cytoplasmic domain that are capable of supporting T cell activation; and (b) introduction of the CRISPR guide sequences and delivery of the CRISPR guided DNA modification enzyme disrupts expression of T cell receptor and/or MHC class I.

12. A vector that comprises a 3′ LTR comprising one or more promoter sequences operably linked to a sequence encoding one or more CRISPR guide sequences, optionally wherein the vector is a viral vector, preferably a lentiviral vector.

13. The vector according to claim 12, wherein the LTR comprises a H1 promoter sequence and/or one a U6 promoter sequence.

14. The vector according to claim 12 or 13, wherein the LTR comprises two or more sequences encoding a CRISPR guide sequence, each operably linked to a different promoter sequence.

15. The vector according to any one of claims 12 to 14, further comprising a nucleic acid sequence that encodes:
(a) a FcR that comprises (I) an extracellular domain that is capable of binding to a constant domain of an antibody and (II) a transmembrane domain and a cytoplasmic domain that are capable of supporting T cell activation;
(b) a CAR, optionally wherein the CAR is specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX;
(c) a recombinant TCR (rTCR);
(d) a restriction factor, optionally wherein the restriction factor is TRIM5CypA;
(e) a suicide gene; and/or
(f) a normal variant of a gene, wherein the sequence encoding one or more CRISPR guide sequences is capable of disrupting expression of a gain-of-function mutant allle of the gene, optionally wherein the gene is STAT1, STAT3, CXCR4, NFKB1A, CARD11, CARD15, STING, NLRP3, NLRC4, PSTPIP1, PIK3CD or PIK3R1.

16. The vector according to any one of claims 12 to 15, wherein one or more of the CRISPR guide sequences is specific for:
(a) a locus controlling the expression of the TCR−CD3 complex;
(b) the TRAC locus, TCR beta constant locus or CD3 locus;
(c) a locus controlling the expression of the MHC class 1;
(d) the TAP2, CIITA, RFX5, RFXAP or RFXANK, β₂m or TAP1 locus;
(e) a locus controlling a checkpoint inhibitor pathway;
(f) a locus associated with a gain of function mutation;
(g) a locus controlling transgene silencing pathway;
(h) the locus controlling expression of CD52; and/or
(i) a locus controlling the expression of an antigen targeted by a CAR, chimeric FcR or monoclonal antibody expressed by the cell(s).

17. Use of the vector according to any one of claims 12 to 16 to:
(a) disrupt expression of TCR and/or MHC class 1 in a cell;
(b) introduce a nucleic acid sequence encoding a FcR that comprises (I) an extracellular domain that is capable of binding to a constant domain of an antibody and (II) a transmembrane domain and a cytoplasmic domain that are capable of supporting T cell activation into a cell;
(c) introduce a nucleic acid sequence encoding a CAR into a cell, optionally wherein the CAR is specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX;
(d) introduce a nucleic acid sequence encoding a CAR into a cell and to disrupt expression of TCR and/or MHC class 1 in the cell, optionally wherein the CAR is specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX;
(e) introduce a nucleic acid sequence encoding a rTCR into a cell and to disrupt expression of TCR in the cell;
(f) introduce a nucleic acid sequence encoding a restriction factor into a cell and to disrupt expression of CCR5 in the cell, optionally wherein the restriction factor is TRIM5CypA;
(g) disrupt expression of a locus controlling a gain of function mutation in a cell and to introduce a nucleic acid sequence encoding a replacement protein into the cell;
(h) disrupt expression of a locus controlling a transgene silencing pathway in a cell, optionally wherein the vector comprises a nucleic acid sequence encoding a transgene silenced by the pathway; or
(i) disrupt expression of a locus controlling a checkpoint inhibitor pathway in a cell and to introduce a nucleic acid sequence encoding a suicide into the cell.

18. The method of any one of claims 1 to 11 or the use of claim 17, wherein the cell is a peripheral blood lymphocyte or a hematopoietic stem cell.

19. The method of any one of claim 1 to 11 or 18 or the use of claim 17 or 18, wherein the cell is allogeneic to an individual into which it is to be administered or autologous to an individual into which it is to be administered.

20. The method of any one of claims 1 to 11 or use of claim 17, wherein the cell is a cord blood T cell, optionally wherein the cord blood T cell is generated by:
(a) providing a sample of cord blood; and
(b) separating cells that express CD62L from the sample wherein the cells that express CD62L comprise one or more cord blood T cells.

21. The method of any one of claims 1 to 11 or 18 to 20, or the vector of any one of claims 12 to 16, wherein:
(a) the vector comprises a sequence encoding a CAR, optionally wherein the CAR is specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX; and/or
(b) one or more of the CRISPR guide sequences is specific for (i) a locus controlling the expression of the TCR−CD3 complex, optionally wherein one or more of the CRISPR guide sequences is specific for the TRAC locus or (ii) a locus controlling the expression of MHC class 1, optionally wherein one or more of the CRISPR guide sequences is specific for the β2M locus.

22. A method for generating T cells that comprise a nucleic acid sequence encoding a CAR and have disrupted TCR and/or MHC class 1 expression, comprising:
(a) providing one or more T cells;
(b) introducing into one or more of said T cells of (a) a nucleic acid sequence encoding a CAR; and
(c) disrupting expression of TCR and/or MHC class 1 in said T cells of (b),
wherein, in (c), the expression of TCR and/or MHC class 1 is disrupted by:
(i) introducing one or more CRISPR guide sequences to said T cells of (b) using a vector that comprises a 3′ long terminal repeat region (LTR) comprising one or more promoter sequences operably linked to the sequence encoding said CRISPR guide sequence(s); and
ii) separately delivering a CRISPR guided DNA modification enzyme to said T cells of (b) by introducing into them a nucleic acid or protein sequence encoding said CRISPR guided DNA modification enzyme.

23. The method of claim 22, wherein the LTR comprises a H1 promoter sequence and/or a U6 promoter sequences.

24. The method of claim 22 or 23, wherein the LTR comprises two or more sequences encoding a CRISPR guide sequence, each operably linked to a different promoter sequence.

25. The method of any one of claims 22 to 24:
(a) the CAR is specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX;
(b) one or more of the CRISPR guide sequences are specific for TRAC;
(c) one or more of the CRISPR guide sequences are specific for β2M;
(c) the T cells of (a) are cord blood T cells; and/or
(d) the CRISPR guided DNA modification enzyme is a cytidine deaminase or a CRISPR nuclease, optionally Cas9.

26. A T cell that comprises a nucleic acid sequence encoding a CAR and has disrupted TCR and/or MEW class 1 expression.

27. The T cell of claim 26, wherein the T cell is produced according to the method of any one of claims 22 to 25.

28. The T cell of claim 26 or 27 for use in a method of treatment of the human or animal body.

29. The T cell of claim 26 or 27 for use in a method of treating a neoplastic condition, an autoimmune condition, an infectious condition, an inflammatory condition, a haematological disorder or a metabolic condition.

30. A method of treating a neoplastic condition, an autoimmune condition, an infectious condition, an inflammatory condition, a haematological disorder or a metabolic condition in a patient in need thereof, the method comprising administering to the patient an effective number of T cells according to claim 26 or 27.

31. The T cell for use of claim 29 or the method of claim 30, wherein the neoplastic condition is a cancer or tumour, optionally wherein the cancer is leukaemia and further optionally wherein the leukaemia is acute lymphoblastic leukaemia, acute myeloid leukaemia, chronic lymphocytic leukaemia or chronic myeloid leukaemia.

32. Use of the vector of claim 21 to:
(a) disrupt expression of TCR in a cell;
(b) disrupt expression of TRAC in a cell;
(c) disrupt expression of MEW class 1 in a cell;
(d) disrupt expression of β2M in a cell;
(e) introduce a nucleic acid sequence encoding CAR into a cell, optionally wherein the CAR is specific for CD10, CD19, CD20, CD22, CD30, CD33, CD45, CD123, erb-B2, CEA, IL13R, Ror, kappa light chain, TCR-beta constant 1, TCR-beta constant 2, MAGE-A1, MUC1, PSMA, VEGF-R, Her2, or CAIX;
(f) introduce a nucleic acid sequence encoding CAR specific for CD19 into a cell and to disrupt expression of TCR and/or β2M in the cell;
(g) introduce a nucleic acid sequence encoding CAR specific for CD20 into a cell and to disrupt expression of TCR and/or β2M in the cell;
(h) introduce a nucleic acid sequence encoding CAR specific for CD123 into a cell and to disrupt expression of TCR and/or β2M in the cell.

33. The use of claim 32, wherein the cell is T cell, optionally a cord blood T cell.

34. A pharmaceutical composition comprising a T cell according to claim 26 or 27.

35. A method for generating universal antibody dependent cord T cells (U-ACTs), comprising:
(a) providing a sample of cord blood;
(b) separating cells that express CD62L from the sample, wherein the cells that express CD62L comprise cord blood T cells;
(c) introducing into one or more of said cord blood T cells of (b) a nucleic acid sequence encoding an Fc-Receptor (FcR) that comprises (I) an extracellular domain that is capable of binding to a constant domain of an antibody and (II) a transmembrane domain and a cytoplasmic domain that are capable of supporting T cell activation; and
(d) disrupting expression of T cell receptor and MHC class I in said cord blood T cells of (c),
wherein, in (d), the expression of T cell receptor and/or MHC class 1 is disrupted by:
(i) introducing one or more CRISPR guide sequences to said cord blood T cells of (c) using a vector that comprises a 3′ long terminal repeat region (LTR) comprising one or more promoter sequences operably linked to the sequence encoding said CRISPR guide sequence(s); and
ii) separately delivering a CRISPR guided DNA modification enzyme to said cord blood T cells of (c) by introducing into them a nucleic acid or protein sequence encoding said CRISPR guided DNA modification enzyme.
